SEQUENCE LISTING (25 sequences)

SEQ ID NO: 1 - DNA, 16 bp, Artificial Sequence
  Description of Artifical Sequence Synthetic oligonucleotide
ctcaatccatggcagc

SEQ ID NO: 2 - DNA, 21 bp, Artificial Sequence
  Description of Combined DNA/RNA Molecule Synthetic oligonucleotide
gcaugcggccucuguuugatt

SEQ ID NO: 3 - DNA, 21 bp, Artificial Sequence
  Description of Combined DNA/RNA Molecule Synthetic oligonucleotide
ucaaacagaggccgcaugctt

SEQ ID NO: 4 - DNA, 19 bp, Artificial Sequence
  Description of Artifical Sequence Synthetic oligonucleotide
tctcccagcgtgcgcccat

SEQ ID NO: 5 - DNA, 16 bp, Artificial Sequence
  Description of Artifical Sequence Synthetic oligonucleotide
tggcaagcatcctgta

SEQ ID NO: 6 - DNA, 16 bp, Artificial Sequence
  Description of Artifical Sequence Synthetic oligonucleotide
tagcctgtcacttctc

SEQ ID NO: 7 - DNA, 16 bp, Artificial Sequence
  Description of Artifical Sequence Synthetic oligonucleotide
gctccagacatcactc

SEQ ID NO: 8 - DNA, 16 bp, Artificial Sequence
  Description of Artifical Sequence Synthetic oligonucleotide
agccattcattccacc

SEQ ID NO: 9 - DNA, 16 bp, Artificial Sequence
  Description of Artifical Sequence Synthetic oligonucleotide
ttattgtgcatctcag

SEQ ID NO: 10 - DNA, 16 bp, Artificial Sequence
  Description of Artifical Sequence Synthetic oligonucleotide
cgtgtatttccgcgtg

SEQ ID NO: 11 - DNA, 16 bp, Artificial Sequence
  Description of Artifical Sequence Synthetic oligonucleotide
ggcacagccagtggcg

SEQ ID NO: 12 - DNA, 16 bp, Artificial Sequence
  Description of Artifical Sequence Synthetic oligonucleotide
cccaaggcactgcaga

SEQ ID NO: 13 - DNA, 16 bp, Artificial Sequence
  Description of Artifical Sequence Synthetic oligonucleotide
accaagtttcttcagc

SEQ ID NO: 14 - DNA, 16 bp, Artificial Sequence
  Description of Artifical Sequence Synthetic oligonucleotide
ctccttggtgcagtct

SEQ ID NO: 15 - DNA, 15 bp, Artificial Sequence
  Description of Artifical Sequence Synthetic oligonucleotide
tcagattcaaaccca

SEQ ID NO: 16 - DNA, 16 bp, Artificial sequence
  Description of Artifical Sequence Synthetic oligonucleotide
gtgttctacaccatta

SEQ ID NO: 17 - protein, 12 aa, Artificial sequence
  Description of Artifical Sequence Synthetic peptide
CYGRKKRRQRRR

SEQ ID NO: 18 - protein, 10 aa, Artificial sequence
  Description of Artifical Sequence Synthetic peptide
CRRRRRRRRR

SEQ ID NO: 19 - protein, 23 aa, Artificial sequence
  Description of Artifical Sequence Synthetic peptide
CYGRKKRRQRRRYGRKKRRQRRR

SEQ ID NO: 20 - protein, 4 aa, Artificial sequence
  Description of Artifical Sequence Synthetic peptide
RGDC

SEQ ID NO: 21 - protein, 5 aa, Artificial sequence
  Description of Artifical Sequence Synthetic peptide
RGDFC

SEQ ID NO: 22 - protein, 5 aa, Artificial sequence
  Description of Artifical Sequence Synthetic peptide
RGDFK

SEQ ID NO: 23 - protein, 19 aa, Artificial sequence
  Description of Artifical Sequence Synthetic peptide
CYGRKKRRQRRRGGGRGDS

SEQ ID NO: 24 - protein, 9 aa, Artificial sequence
  Description of Artifical Sequence Synthetic peptide
RRRRRRRRR

SEQ ID NO: 25 - DNA, 16 bp, Artificial Sequence
  Description of Artifical Sequence Synthetic oligonucleotide
tagcttgtcccatctc

(19) United States
(12) Patent Application Publication
(10) Pub. No.: US 2011/0305770 A1
(43) Pub. Date: Dec. 15, 2011

(54) RELEASABLE POLYMERIC LIPIDS FOR NUCLEIC ACIDS DELIVERY SYSTEM

(75) Inventors: Hong Zhao, Edison, NJ (US); Weili Yan, Piscataway, NJ (US); Lianjun Shi, Bridgewater, NJ (US); Dechun Wu, Bridgewater, NJ (US)

(73) Assignee: ENZON PHARMACEUTICALS, INC., Bridgewater, NJ (US)

(21) Appl. No.: 13/129,628

(22) PCT Filed: Nov. 17, 2009
(86) PCT No.: PCT/US09/64701

§ 371 Date: Aug. 30, 2011

Related U.S. Application Data
(60) Provisional application No. 61/115,379, filed on Nov. 17, 2008.
(60) Provisional application No. 61/115,371, filed on Nov. 17, 2008.

(51) Int. Cl. A61K 9/14; C07C 237/32; C07F 9/24; C07F 9/09; C07D 207/09; C07K 5/11; C07D 475/04; C07K 14/475; C07K 14/79; C07K 14/655; C07K 14/68; C07K 14/745; A61K 47/44; A61K 31/7088; A61K 31/713; A61K 38/02; C12N 5/09; A61P 35/00; A61P 31/12; A61P 29/00; C07C 271/20; B82Y 5/00

(52) U.S. Cl. 424/499; 554/57; 554/41; 554/106; 554/84; 548/531; 530/331; 544/261; 530/399; 530/394; 530/311; 530/312; 530/381; 514/785; 514/788; 514/44 R; 514/44 A; 514/20.9; 435/375; 977/773; 977/800; 977/906

(57) ABSTRACT

The present invention relates to polymer conjugated releasable lipids and nanoparticle compositions containing the same for the delivery of nucleic acids and methods of modulating gene expression using the same. In particular, this invention relates to releasable polymeric lipids containing an acid-labile linker based on a ketal or acetal-containing linker, or an imine-containing linker.

### CROSS-REFERENCE TO RELATED APPLICATION

This application claims the benefit of priority from U.S. Provisional Patent Application Ser. Nos. 61/115,371 and 61/115,379 filed Nov. 17, 2008, the contents of each of which are incorporated herein by reference.

### BACKGROUND OF THE INVENTION

Therapy using nucleic acids has been proposed for treating various diseases. One such proposed nucleic acid therapy is antisense therapy, wherein therapeutic genes can selectively modulate gene expression associated with disease and minimize side effects that may be associated with other therapeutic approaches to treating disease.

Therapy using nucleic acids has, however, heretofor been limited due to challenges associated with delivery and stability of such therapeutic nucleic acids. Several gene delivery systems have been proposed to overcome the above-noted challenges and effectively introduce therapeutic genes into a target area, such as cancer cells or other cells or tissues, in vitro and in vivo.

Nevertheless, new delivery systems and methods for delivering nucleic acids for therapeutic purposes are needed, and are provided herein.

### SUMMARY OF THE INVENTION

The present invention provides releasable polymeric lipids containing an acid labile linker, and nanoparticle compositions containing the same for nucleic acids delivery. Polynucleic acids, such as oligonucleotides, are encapsulated within nanoparticle complexes containing a mixture of a releasable polymeric lipid described herein, a cationic lipid, and a fusogenic lipid.

In accordance with this aspect of the invention, the releasable polymeric lipids for the delivery of nucleic acids (i.e., an oligonucleotide) have Formula (I):

R-(L₁)ₐ-M-(L₂)_{b}-Q

wherein

R is a non-antigenic polymer;

L₁₋₂ are independently selected bifunctional linkers;

M is an acid labile linker;

Q is a substituted or unsubstituted saturated or unsaturated C4-30-containing moiety;

(a) is zero or a positive integer; and

(b) is zero or a positive integer,

wherein a targeting group is optionally linked to the non-antigenic polymer.

The present invention also provides nanoparticle compositions for nucleic acids delivery. According to the present invention, the nanoparticle composition for the delivery of nucleic acids (i.e., an oligonucleotide) includes:

(i) a cationic lipid;

(ii) a fusogenic lipid; and

(iii) a compound of Formula (I).

In another aspect of the present invention, there are provided methods of delivering nucleic acids (preferably oligonucleotides) to a cell or tissue, in vivo and in vitro. Oligonucleotides introduced by the methods described herein can modulate expression of a target gene.

In a further aspect, the present invention provides methods of inhibiting expression of a target gene, i.e., oncogenes and genes associated with inflammation disease in mammals, preferably humans. The methods include contacting cells such as cancer cells or tissues with a nanoparticle prepared from the nanoparticle composition described herein. The oligonucleotides encapsulated within the nanoparticle are released and mediate down-regulation of mRNA or protein in the cells or tissues being treated. The treatment with the nanoparticle allows modulation of target gene expression and the attendant benefits associated therewith in the treatment of disease, such as inhibition of the growth of cancer cells. Such therapies can be carried out as a single treatment or as a part of combination therapy, with one or more useful and/or approved treatments.

Further aspects include methods of making the compounds of Formula (I) as well as nanoparticles containing the same.

The releasable polymeric lipids described herein include an acid labile linker. As the nanoparticles containing the biologically active moieties reach the target site, e.g., intracellular or extracellular environments of acid pH, the releasable polymeric lipids start to degrade, rupturing the nanoparticle, and releasing the therapeutics at and/or within the target site. By employing a ketal or acetal-containing moiety or an imine-containing moiety, the nanoparticles can retain stability in neutral or slightly basic conditions. However, at the usual low pH target site, such as tumor cells, ketal and acetal moieties degrade, thereby releasing encapsulated therapeutics such as oligonucleotides.

The nanoparticle containing the releasable polymeric lipids helps dissociate and release the nucleic acids encapsulated therein when the nanoparticle enters the cells and reaches an acidic cellular compartment, such as endosome. Without being bound by any theory, such a feature is attributed in part to the acid labile linker. The ketal or imine-based linkers are acid-labile and hydrolyzed in acidic environment such as an endosome. The linkers facilitate disruption of the nanoparticle and endosome, thereby allowing intracellular release of nucleic acids.

One advantage of the present invention is that the nanoparticle compositions containing the releasable polymeric lipids described herein provide a means for the delivery of nucleic acids in vitro, as well as for in vivo administration of nucleic acids. This delivery technology allows enhanced stability, transfection efficiency, and bioavailability of therapeutic oligonucleotides in the body.

The releasable polymeric lipids extend circulation of nanoparticles and prevent premature excretion of nanoparticles from the body. The polymeric lipids also reduce immunogenicity.

The releasable polymeric lipids described herein stabilize nanoparticle complexes and nucleic acids therein in biological fluids. Without being bound by any theory, it is believed that the nanoparticle complex enhances stability of the nucleic acids so encapsulated, and at least in part shields the nucleic acids from nucleases, thereby protecting the encapsulated nucleic acids from degradation in the presence of, e.g., blood or tissues.

The nanoparticles described herein also advantageously provide, e.g., a higher transfection efficiency. The nanoparticles described herein allow the transfection of cells in vitro and in vivo without the aid of a transfection agent. The nanoparticles are safe because they do not have the same toxicity effect as art-known nanoparticles, which require transfection agents. The high transfection efficiency of the nanoparticles also provides an improved means to deliver therapeutic nucleic acids into the cytoplasm and nucleus in the cells.

The nanoparticles described herein also advantageously provide stability and flexibility in the preparation of the nanoparticles. The nanoparticles can be prepared in a wide range of pH, such as from about 2 through about 12. The nanoparticles described herein also may be used clinically at a desirable physiological pH, such as from about 7.2 through about 7.6.

The nanoparticle delivery systems described herein also allow sufficient amounts of the therapeutic oligonucleotides to be selectively available at the desired target area, such as cancer cells via EPR (Enhanced Permeation and Retention) effects. The nanoparticle compositions thus improve specific mRNA down regulation in cancer cells or tissues.

Another advantage is that the releasable polymeric lipids described herein allow preparation of nanoparticles in homogenous size. The nanoparticle complexes containing the releasable polymeric lipids described herein are stable under buffer conditions.

Yet another advantage is that the nanoparticles described herein allow delivery of biologically active molecules, such as small molecule chemotherapeutics of one or more different target oligonucleotides, thereby attaining synergistic effects in the treatment of disease.

Other and further advantages will be apparent from the following description.

For purposes of the present invention, the term “residue” shall be understood to mean that portion of a compound, to which it refers, e.g., polyethylene glycol, etc. that remains after it has undergone a substitution reaction with another compound.

For purposes of the present invention, the term “alkyl” refers to a saturated aliphatic hydrocarbon, including straight-chain, branched-chain, and cyclic alkyl groups. The term “alkyl” also includes alkyl-thio-alkyl, alkoxyalkyl, cycloalkylalkyl, heterocycloalkyl, and C₁₋₆ alkylcarbonylalkyl groups. Preferably, the alkyl group has 1 to 12 carbons. More preferably, it is a lower alkyl of from about 1 to 7 carbons, yet more preferably about 1 to 4 carbons. The alkyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “substituted” refers to adding or replacing one or more atoms contained within a functional group or compound with one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ alkylcarbonylalkyl, aryl, and amino groups.

For purposes of the present invention, the term “alkenyl” refers to groups containing at least one carbon-carbon double bond, including straight-chain, branched-chain, and cyclic groups. Preferably, the alkenyl group has about 2 to 12 carbons. More preferably, it is a lower alkenyl of from about 2 to 7 carbons, yet more preferably about 2 to 4 carbons. The alkenyl group can be substituted or unsubstituted. When substituted the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “alkynyl” refers to groups containing at least one carbon-carbon triple bond, including straight-chain, branched-chain, and cyclic groups. Preferably, the alkynyl group has about 2 to 12 carbons. More preferably, it is a lower alkynyl of from about 2 to 7 carbons, yet more preferably about 2 to 4 carbons. The alkynyl group can be substituted or unsubstituted. When substituted the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups. Examples of “alkynyl” include propargyl, propyne, and 3-hexyne.

For purposes of the present invention, the term “aryl” refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl. For purposes of the present invention, the term “cycloalkyl” refers to a C₃₋₈ cyclic hydrocarbon. Examples of cycloalkyl include cyclopropyl, cyclobutyl, cyclopentyl, cyclohexyl, cycloheptyl and cyclooctyl.

For purposes of the present invention, the term “cycloalkenyl” refers to a C₃₋₈ cyclic hydrocarbon containing at least one carbon-carbon double bond. Examples of cycloalkenyl include cyclopentenyl, cyclopentadienyl, cyclohexenyl, 1,3-cyclohexadienyl, cycloheptenyl, cycloheptatrienyl, and cyclooctenyl.

For purposes of the present invention, the term “cycloalkylalkyl” refers to an alklyl group substituted with a C₃₋₈ cycloalkyl group. Examples of cycloalkylalkyl groups include cyclopropylmethyl and cyclopentylethyl.

For purposes of the present invention, the term “alkoxy” refers to an alkyl group of indicated number of carbon atoms attached to the parent molecular moiety through an oxygen bridge. Examples of alkoxy groups include, for example, methoxy, ethoxy, propoxy and isopropoxy.

For purposes of the present invention, an “alkylaryl” group refers to an aryl group substituted with an alkyl group.

For purposes of the present invention, an “aralkyl” group refers to an alkyl group substituted with an aryl group.

For purposes of the present invention, the term “alkoxyalkyl” group refers to an alkyl group substituted with an alkloxy group.

For purposes of the present invention, the term “alkyl-thio-alkyl” refers to an alkyl-S-alkyl thioether, for example methylthiomethyl or methylthioethyl.

For purposes of the present invention, the term “amino” refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

For purposes of the present invention, the term “alkylcarbonyl” refers to a carbonyl group substituted with alkyl group.

For purposes of the present invention, the term “halogen’ or “halo” refers to fluorine, chlorine, bromine, and iodine.

For purposes of the present invention, the term “heterocycloalkyl” refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrrolidinyl.

For purposes of the present invention, the term “heteroaryl” refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

For purposes of the present invention, the term “heteroatom” refers to nitrogen, oxygen, and sulfur.

In some embodiments, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aryls include moieties such as napthyl; substituted aryls include moieties such as 3-bromo phenyl; aralkyls include moieties such as tolyl; heteroalkyls include moieties such as ethylthiophene; substituted heteroaryls include moieties such as 3-methoxythiophene; alkoxy includes moieties such as methoxy; and phenoxy includes moieties such as 3-nitrophenoxy. Halo shall be understood to include fluoro, chloro, iodo and bromo.

For purposes of the present invention, “positive integer” shall be understood to include an integer equal to or greater than 1 and as will be understood by those of ordinary skill to be within the realm of reasonableness by the artisan of ordinary skill.

For purposes of the present invention, the term “linked” shall be understood to include covalent (preferably) or noncovalent attachment of one group to another, i.e., as a result of a chemical reaction.

The terms “effective amounts” and “sufficient amounts” for purposes of the present invention shall mean an amount which achieves a desired effect or therapeutic effect as such effect is understood by those of ordinary skill in the art.

The term “nanoparticle” and/or “nanoparticle complex” formed using the nanoparticle composition described herein refers to a lipid-based nanocomplex. The nanoparticle contains nucleic acids such as oligonucleotides encapsulated in a mixture of a cationic lipid, a fusogenic lipid, and a PEG lipid. Alternatively, the nanoparticle can be formed without nucleic acids.

For purposes of the present invention, the term “therapeutic oligonucleotide” refers to an oligonucleotide used as a pharmaceutical or diagnostic.

For purposes of the present invention, “modulation of gene expression” shall be understood as broadly including down-regulation or up-regulation of any types of genes, preferably associated with cancer and inflammation, compared to a gene expression observed in the absence of the treatment with the nanoparticle described herein, regardless of the route of administration.

For purposes of the present invention, “inhibition of expression of a target gene” shall be understood to mean that mRNA expression or the amount of protein translated are reduced or attenuated when compared to that observed in the absence of the treatment with the nanoparticle described herein. Suitable assays of such inhibition include, e.g., examination of protein or mRNA levels using techniques known to those of skill in the art such as dot blots, northern blots, in situ hybridization, ELISA, immunoprecipitation, enzyme function, as well as phenotypic assays known to those of skill in the art. The treated conditions can be confirmed by, for example, decrease in mRNA levels in cells, preferably cancer cells or tissues.

Broadly speaking, successful inhibition or treatment shall be deemed to occur when the desired response is obtained. For example, successful inhibition or treatment can be defined by obtaining e.g, 10% or higher (i.e. 20% 30%, 40%) downregulation of genes associated with tumor growth inhibition. Alternatively, successful treatment can be defined by obtaining at least 20% or preferably 30%, more preferably 40% or higher (i.e., 50% or 80%) decrease in oncogene mRNA levels in cancer cells or tissues, including other clinical markers contemplated by the artisan in the field, when compared to that observed in the absence of the treatment with the nanoparticle described herein.

Further, the use of singular terms for convenience in description is in no way intended to be so limiting. Thus, for example, reference to a composition comprising an oligonucleotide, a cholesterol analog, a cationic lipid, a fusogenic lipid, a releasable polymeric lipid of Formula (I), a PEG lipid etc. refers to one or more molecules of that oligonucleotide, cholesterol analog, cationic lipid, fuosogenic lipid, releasable polymeric lipid, PEG lipid, etc. It is also contemplated that the oligonucleotide can be the same or different kind of gene. It is also to be understood that this invention is not limited to the particular configurations, process steps, and materials disclosed herein as such configurations, process steps, and materials may vary somewhat.

It is also to be understood that the terminology employed herein is used for the purpose of describing particular embodiments only and is not intended to be limiting, since the scope of the present invention will be limited by the appended claims and equivalents thereof.

### BRIEF DESCRIPTION OF THE DRAWINGS

FIG. 1 schematically illustrates a reaction scheme for preparing compound 3, as described in Examples 6-8.

FIG. 2 schematically illustrates a reaction scheme for preparing compound 10, as described in Examples 9-14.

FIG. 3 schematically illustrates a reaction scheme for preparing compound 17, as described in Examples 15-21.

FIG. 4 schematically illustrates a reaction scheme for preparing compound 22, as described in Examples 22-26.

FIG. 5 schematically illustrates a reaction scheme for preparing compound 26, as described in Examples 27-28.

FIG. 6 schematically illustrates a reaction scheme for preparing compound 30, as described in Examples 29-30.

FIG. 7 schematically illustrates a reaction scheme for preparing compound 32, as described in Examples 31-32.

FIG. 8 schematically illustrates a reaction scheme for preparing compound 38, as described in Examples 33-37.

FIG. 9 schematically illustrates a reaction scheme for preparing compound 44, as described in Examples 38-43.

FIG. 10 schematically illustrates a reaction scheme for preparing compound 46, as described in Examples 44-45.

FIG. 11 schematically illustrates a reaction scheme for preparing compound 52, as described in Examples 46-50.

FIG. 12 describes changes in size of nanoparticles at pH 7.4, as described in Example 52. 0 h is the left bar; 3 h is the middle bar; and 18 h is the right bar in each formulation.

FIG. 13A describes changes in size of nanoparticles at pH 6.5 and 5.5, as described in Example 53.

FIG. 13B describes nanoparticle stability in pH 5.5 buffer, as a function of nanoparticle size.

FIG. 14 describes stability of nanoparticles in mouse plasma, as described in Example 54.

FIG. 15 describes photomicroscopic images of cells demonstrating cellular uptake of and cytoplasmic localization of fluorescent nucleic acids, as described in Example 55.

FIG. 16 describes effects of increase in amounts of releasable polymeric lipids on modulation of target gene expression, as described in Example 56. From left to right, the bars within each experimental group (NP4, NP5, NP6, NP7) are labelled, respectively, as: 600 nM, 300 nM, 150 nM, 75 nM; and on the far right, a single bar is UTC.

FIG. 17 describes BCL2 mRNA knockdown by siRNA encapsulated within nanoparticles described herein in 15PC3 cells, as described in Example 57. The bars are labelled as follows:

Empty NP: left bar is 200 n, right bar is 100 nM;

2% rPEG: from left to right: 200 nM, 100 nM, 50 nM, 25 nM;

5% rPEG: from left to right: 200 nM, 100 nM, 50 nM, 25 nM;

8% rPEG: from left to right: 200 nM, 100 nM, 50 nM, 25 nM;

Scrambled: from left to right: 200 nM, 100 nM, 50 nM, 25 nM;

Mock, as indicated;

UTC, as indicated; and Bcl2_Tfx: from left to right: 200 nM, 25 nM, 10 nM, 100 nM.

FIG. 18 describes BCL2 mRNA knockdown by siRNA encapsulated within nanoparticles as described herein in A549 cells, as described in Example 58. The bars are labelled as follows:

UT: A549;

NP-1: from left to right: 200 nM, 100 nM, 50 nM, 25 nM, 12.5 nM;

NP-2: from left to right: 200 nM, 100 nM, 50 nM, 25 nM, 12.5 nM;

NP-3: from left to right: 200 nM, 100 nM, 50 nM, 25 nM, 12.5 nM;

NP-SCR: from left to right: 200 nM, 100 nM, 50 nM, 25 nM, 12.5 nM; and

Bcl2 siRNA T: from left to right: 12.5 nM, 4 nM, 0.8 nM, 0.16 nM, 0.03 nM, A549T.

FIG. 19 describes ErbB3 mRNA knockdown by oligonucleotides including LNA in DU149 cells, as described in Example 59. The bars are labelled as follows:

A: from left to right: 1000 nM, 500 nM, 250 nM, 125 nM, 62 nM, 0 nM;

B: from left to right: 1000 nM, 500 nM, 250 nM, 125 nM, 62 nM, 0 nM;

C: from left to right: 1000 nM, 500 nM, 250 nM, 125 nM, 62 nM, 0 nM;

D: from left to right: 1000 nM, 500 nM, 250 nM, 125 nM, 62 nM, 0 nM; and

E: from left to right: 1000 nM, 500 nM, 250 nM

F: from left to right: 125 mM, 62 nM, 0 nM.

### DETAILED DESCRIPTION OF THE INVENTION

### A. Overview

### 1. Releasable Polymeric Lipids of Formula (I)

In one aspect of the present invention, there are provided releasable polymeric lipids of Formula (I):

R-(L₁)ₐ-M-(L₁)_{b}-Q

wherein

R is a non-antigenic polymer;

L₁₋₂ are independently selected bifunctional linkers;

M is an acid labile linker;

Q is a substituted or unsubstituted saturated or unsaturated C4-30-containing moiety;

(a) is zero or a positive integer, preferably zero or an integer of from about 1 to about 10 (e.g., 1, 2, 3, 4, 5, 6); and

(b) is zero or a positive integer, preferably zero or an integer of from about 1 to about 10 (e.g., 1, 2, 3, 4, 5, 6),

wherein a targeting group is optionally linked to the non-antigenic polymer.

L₁ and L₂ are independently the same or different when (a) and (b) are equal to or greater than 2.

According to the present invention, the compounds of Formula (I) described herein include the Q hydrocarbon group (aliphatic). The Q group has Formula (Ia):

(Ia)

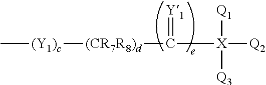

wherein

Y₁ is O, S or NR₃₁, preferably O or NR₃₁;

Y′₁ is O, S, or NR₃₁, preferably O;

(c) is 0 or 1;

(d) is 0 or a positive integer, preferably zero or an integer of from about 1 to about 10 (e.g., 1, 2, 3, 4, 5, 6);

(e) is 0 or 1;

X is C, N or P;

Q₁ is H, C₁₋₃ alkyl, NR₃₂, OH, or

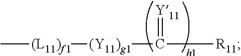

Q₂ is H, C₁₋₃ alkyl, NR₃₃, OH, or

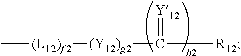

Q₃ is a lone electron pair, (═O), H, C₁₋₃ alkyl, NR₃₄, OH, or

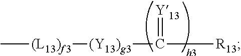

provided that
(i) when X is C, Q₃ is not a lone electron pair or (═O);
(ii) when X is N, Q₃ is a lone electron pair; and
(iii) when X is P, Q₃ is Q₃ is (═O) and (e) is 0,
   wherein
   L₁₁, L₁₂ and L₁₃ are independently selected bifunctional spacers;
   Y₁₁, Y₁₂ and Y₁₃ are independently O, S or NR₃₅, preferably O or NR₃₅;
   Y′₁₁ Y′₁₂, Y′₁₃ are independently O, S or NR₃₅, preferably O;
   R₁₁, R₁₂ and R₁₃ are independently saturated or unsaturated C₄₋₃₀;
   (f1), (f2) and (f3) are independently 0 or 1;
   (g1), (g2) and (g3) are independently 0 or 1; and
   (h1), (h2) and (h3) are independently or 1;

R₇₋₈ are independently selected from among hydrogen, hydroxyl, amine, substituted amine, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, and substituted C₁₋₆ heteroalkyl, preferably hydrogen, methyl, ethyl and propyl;

R₃₁₋₃₅ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, and substituted C₁₋₆ heteroalkyl, preferably hydrogen, methyl, ethyl and propyl,

provided that Q includes at least one or two (e.g. one, two, three) of R₁₁, R₁₂ and R₁₃.

Preferably, Q includes at least two of R₁₁, R₁₂ and R₁₃.

C(R₇)(R₈), in each occurrence, is the same or different when (d) is equal to or greater than 2.

The combinations of the bifunctional linkers and the bifunctional spacers contemplated within the scope of the present invention include those in which combinations of variables and substituents of the linker and spacer groups are permissible so that such combinations result in stable compounds of Formula (I). For example, the combinations of values and substituents do not permit oxygen, nitrogen or carbonyl to be positioned directly adjacent to S—S or imine.

In one preferred embodiment, Y′₁ is oxygen.

In another preferred embodiment, Y′₁₁, Y′₁₂ and Y′₁₃ are oxygen.

In another preferred embodiment, Y₁₁, Y₁₂ and Y₁₃ are independently oxygen or NH.

In one embodiment, (f1), (f2) and (f3) are not simultaneously zero.

In another embodiment, (g1), (g2), (g3), (h1), (h2) and (h3) are not simultaneously zero.

According to the present invention, the releasable polymeric lipids described herein have Formula (II):

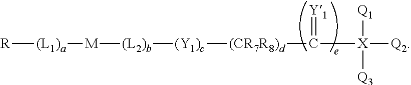

In one preferred aspect, the acid labile linker is a ketal- or acetal-containing moiety or an imine-containing moiety.

The ketal or acetal-containing moiety has the formula:

—CR₃R₄—O—CR₁R₂—O—CR₅R₆—,

wherein

R₁₋₂ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, and substituted arylcarbonyloxy, preferably, hydrogen, methyl, ethyl, propyl; and

R₃₋₆ are independently selected from among hydrogen, amine, substituted amine, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, and substituted arylcarbonyloxy, preferably, hydrogen, methyl, ethyl and propyl.

Preferably, R₁ and R₂ are independently selected from among hydrogen, C₁₋₆ alkyls, C₃₋₈ branched alkyls, C₃₋₈ cycloalkyls, C₁₋₆ substituted alkyls, C₃₋₈ substituted cycloalkyls, aryls, substituted aryls and aralkyls, preferably hydrogen, methyl, ethyl, propyl.

In one preferred embodiment, both R₁ and R₂ are not simultaneously hydrogen.

In another preferred embodiment, R₃₋₆ are independently selected from among hydrogen, C₁₋₆ alkyls, C₃₋₈ branched alkyls, C₃₋₈ cycloalkyls, C₁₋₆ substituted alkyls, C₃₋₈ substituted cycloalkyls, aryls, substituted aryls and aralkyls. More preferably, R₃₋₆ are all hydrogen.

More preferably, R₁ and R₂ are the same or different C₁₋₆ alkyls, saturated or unsaturated such as ethyl, methyl, propyl and butyl. Yet more preferably, both R₁ and R₂ are methyl. In one particular embodiment, the M group is —-CH₂—O—C(CH₃)(CH₃)-G-CH₂—.

In certain embodiments, the releasable polymeric lipids have Formula (IIa):

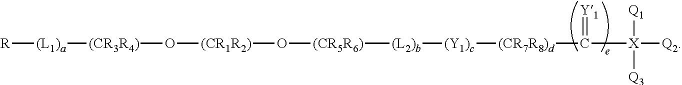

The imine linker has the formula:

—N═CR₁₀— or —CR₁₀═N—,

wherein R₁₀ is hydrogen, C₁₋₆ alkyl, C₃₋₈ branched alkyl, C₃₋₈ cycloalkyl, C₁₋₆ substituted alkyl, C₃₋₈ substituted cycloalkyl, aryl and substituted aryl, preferably hydrogen, alkyl, methyl, or propyl.

Preferably, R₁₀ is hydrogen and the acid-labile linker is —N═CH— or —CH═N—.

In certain embodiments, the releasable polymeric lipids have Formula (IIb) or (II′b):

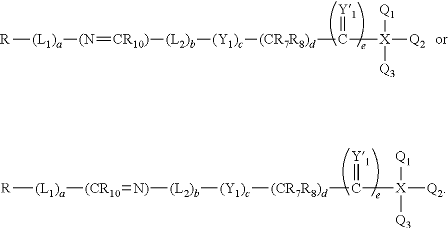

According to the present invention, the releasable polymeric lipids describe herein can include a targeting group. The present invention provides releasable polymeric lipids in which R group, preferably at the terminal, is attached to a targeting group. The releasable polymeric lipids have the formula:

A-R-(L₁)ₐ-M-(L₂)_{b}-Q,

wherein A is a targeting group, preferably a cell surface targeting group.

The targeting group can be attached to the non-antigenic polymer using a linker molecule, such as an amide, amido, carbonyl, ester, peptide, disulphide, silane, nucleoside, abasic nucleoside, polyether, polyamine, polyamide, peptide, carbohydrate, lipid, polyhydrocarbon, phosphate ester, phosphoramidate, thiophosphate, alkylphosphate, maleimidyl linker or photolabile linker. Any known techniques in the art can be used for conjugating a targeting group to the polymer such as polyethylene glycol without undue experimentation. For example, the polymers for conjugation to a targeting group are converted into a suitably activated polymer, using the activation techniques described in U.S. Pat. Nos. 5,122,614 and 5,808,096 and other techniques known in the art without undue experimentation. Examples of activated PEGs useful for conjugating to a targeting group include, but are not limited to, polyethylene glycol-succinate, polyethylene glycol-succinimidyl succinate (PEG-NHS), polyethyleneglycol-acetic acid (PEG-CH₂COOH), polyethylene glycol-amine (PEG-NH₂), polyethylene glycol-maleimide, and polyethylene glycol-tresylate (PEG-TRES).

In certain embodiments, the releasable polymeric lipids have Formula (IIIa):

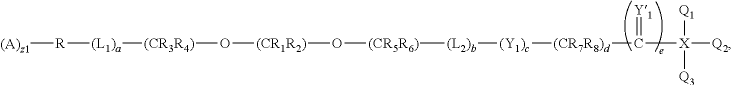

wherein A is a targeting group and (z1) is zero or 1.

In certain embodiments, the releasable polymeric lipids have Formula (IIIb) or (III′b):

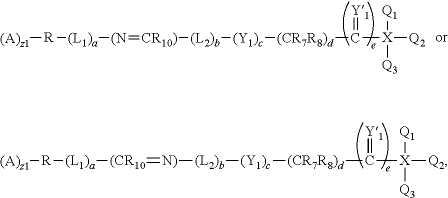

wherein A is a targeting group and (z1) is zero or 1.

### 2. Non-Antigenic Polymer: R Group

Polymers employed in the releasable polymeric lipids described herein are preferably water soluble polymers and substantially non-antigenic such as polyalkylene oxides (PAO's).

In one preferred aspect, the polyalkylene oxide includes polyethylene glycols and polypropylene glycols. More preferably, the polyalkylene oxide includes polyethylene glycol (PEG).

The polyalkylene oxide has a number average molecular weight of from about 200 to about 100,000 daltons, preferably from about 200 to about 20,000 daltons. The polyalkylene oxide can be more preferably from about 500 to about 10,000, and yet more preferably from about 1,000 to about 5,000 daltons. In one particular embodiment, polymeric portion has the total number average molecular weight of about 2,000 daltons.

Preferably, the polyalkylene is a polyethylene glycol with a number average molecular weight ranging from about 200 to about 20,000 daltons, more preferably from about 500 to about 10,000 daltons, yet more preferably from about 1,000 to about 5,000 daltons (i.e., about 1,500 to about 3,000 daltons). In one particular embodiment, the PEG has a molecular weight of about 2,000 daltons. In another particular embodiment, the PEG has a molecular weight of about 750 daltons.

PEG is generally represented by the structure:

—O—(CH₂CH₂O)ₙ—

where (n) is a positive integer from about 5 to about 2300, preferably from about 5 to about 460 so that the polymeric portion of PEG lipid has a number average molecular weight of from about 200 to about 100,000 daltons, preferably from about 200 to about 20,000 daltons. (n) represents the degree of polymerization for the polymer, and is dependent on the molecular weight of the polymer.

Alternatively, the polyethylene glycol (PEG) residue portion can be represented by the structure:

—Y₇₁—(CH₂CH₂O)ₙ—CH₂CH₂Y₇₁—,

—Y₇₁—(CH₂CH₂O)ₙ—CH₂C(═Y₇₂)—Y₇₁—,

—Y₇₁—C(═Y₇₂)—(CH₂)ₐ₁₂—Y₇₃—(CH₂CH₂O)ₙ—CH₂CH₂—Y₇₃—(CH₂)ₐ₁₂—C(═Y₇₂)—Y₇₁— and

—Y₇₁—(CR₇₁R₇₂)ₐ₁₂—Y₇₃—(CH₂)_{b12}—O—(CH₂CH₂O)ₙ—(CH₂)_{b12}—Y₇₃—(CR₇₁R₇₂)ₐ₁₂—Y₇₁—,

wherein:

Y₇₁ and Y₇₃ are independently O, S, SO, SO₂, NR₇₃ or a bond;

Y₇₂ is O, S, or NR₇₄;

R₇₁₋₇₄ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy, preferably hydrogen, methyl, ethyl or propyl;

(a12) and (b12) are independently zero or positive integers, preferably zero or an integer of from about 1 to about 6 (e.g., 1, 2, 3), and more preferably 1; and

(n) is an integer from about 5 to about 2300, preferably from about 5 to about 460.

The terminal end (A′ group) of PEG can end with H, NH₂, OH, CO₂H, C₁₋₆ alkyl (e.g., methyl, ethyl, propyl), C₁₋₆ alkoxy (e.g., methoxy, ethoxy, propyloxy), acyl or aryl. In a preferred embodiment, the terminal hydroxyl group of PEG is substituted with a methoxy or methyl group. In one preferred embodiment, the PEG employed in the PEG lipid is methoxy PEG.

The PEG may be directly conjugated directly to acid labile linkers or via a linker moiety. The polymers for conjugation to an acid labile or a lipid structure are converted into a suitably activated polymer, using the activation techniques described in U.S. Pat. Nos. 5,122,614 and 5,808,096 and other techniques known in the art without undue experimentation.

Examples of activated PEGs useful for the preparation of a PEG lipid include, for example, methoxypolyethylene glycol-succinate, methoxypolyethylene glycol-succinimidyl succinate (mPEG-NHS), methoxypolyethyleneglycol-acetic acid (mPEG-CH₂COOH), methoxypolyethylene glycol-amine (mPEG-NH₂), and methoxypolyethylene glycol-tresylate (mPEG-TRES).

In certain aspects, polymers having terminal carboxylic acid groups can be employed in the PEG lipids described herein. Methods for preparing polymers having terminal carboxylic acids in high purity are described in U.S. patent application Ser. No. 11/328,662, the contents of which are incorporated herein by reference.

In alternative aspects, polymers having terminal amine groups can be employed to make the PEG-lipids described herein. The methods of preparing polymers containing terminal amines in high purity are described in U.S. patent application Ser. Nos. 11/508,507 and 11/537,172, the contents of each of which are incorporated by reference.

In a further aspect of the invention, the polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In yet a further embodiment and as an alternative to PAO-based polymers such as PEG, one or more effectively non-antigenic materials such as dextran, polyvinyl alcohols, carbohydrate-based polymers, hydroxypropylmethacrylamide (HPMA), polyalkylene oxides, and/or copolymers thereof can be used. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose. See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will further realize that the foregoing list is merely illustrative and that all polymeric materials having the qualities described herein are contemplated. For purposes of the present invention, “substantially or effectively non-antigenic” means all materials understood in the art as being nontoxic and not eliciting an appreciable immunogenic response in mammals.

### 3. The Bifunctional Linker: L₁ and L₂ Groups

According to the present invention, the L₁ group as included in the compounds of Formula (I) is selected from among:

—(CR₂₁R₂₂)ₜ₁-[C(═Y₁₆)]ₐ₃—,

—(CR₂₁R₂₂)ₜ₁Y₁₇—(CR₂₃R₂₄)ₜ₂—(Y₁₈)ₐ₂-[C(═Y₁₆)]ₐ₃—,

—(CR₂₁R₂₂CR₂₃R₂₄Y₁₇)ₜ₁—[C(═Y₁₆)]ₐ₃—,

—(CR₂₁R₂₂CR₂₃R₂₄Y₁₇)ₜ₁(CR₂₅R₂₆)ₜ₄—(Y₁₈)ₐ₂-[C(═Y₁₆)]ₐ₃—,

—[(CR₂₁R₂₂CR₂₃R₂₄)ₜ₃Y₁₇]ₜ₃(CR₂₅R₂₆)ₜ₄—(Y₁₈)ₐ₂-[C(═Y₁₆)]ₐ₃—,

—(CR₂₁R₂₂)ₜ₁-[(CR₂₃R₂₄)ₜ₂Y₁₇]ₜ₃(CR₂₅R₂₆)ₜ₄—(Y₁₈)ₐ₂-[C(═Y₁₆)]ₐ₃—,

—(CR₂₁R₂₂)ₜ₁(Y₁₇)ₐ₂[C(═Y₁₆)]ₐ₃(CR₂₃R₂₄)ₜ₂—,

—(CR₂₁R₂₂)ₜ₁(Y₁₇)ₐ₂[C(═Y₁₆)]₁₃Y₁₄(CR₂₃R₂₄)ₜ₂—,

—(CR₂₁R₂₂)ₜ₁(Y₁₇)ₐ₂[C(═Y₁₆)]ₐ₃(CR₂₃R₂₄)ₜ₂—Y₁₅—(CR₂₃R₂₄)ₜ₃—,

—(CR₂₁R₂₂)ₜ₁(Y₁₇)ₐ₂[C(═Y₁₆)]ₐ₃Y₁₄(CR₂₃R₂₄)ₜ₂—Y₁₅—(CR₂₃R₂₄)ₜ₃—,

—(CR₂₁R₂₂)ₜ₁(Y₁₇)ₐ₂[C(═Y₁₆)]ₐ₃(CR₂₃R₂₄CR₂₅R₂₆Y₁₉)ₜ₂(CR₂₇CR₂₈)ₜ₃—,

—(CR₂₁R₂₂)ₜ₁(Y₁₇)ₐ₂[C(═Y₁₆)]ₐ₃Y₁₄(CR₂₃R₂₄CR₂₅R₂₆Y₁₉)ₜ₂(CR₂₇CR₂₈)ₜ₃—, and

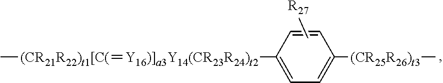

wherein:

Y₁₆ is O, NR₂₈, or S, preferably oxygen;

Y₁₄₋₁₅ and Y₁₇₋₁₉ are independently O, NR₂₉, or S, preferably O, or NR₂₉;

R₂₁₋₂₇ are independently selected from among hydrogen, hydroxyl, amine, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy, preferably, hydrogen, methyl, ethyl or propyl; and

R₂₈₋₂₉ are independently selected from among hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy, preferably, hydrogen, methyl, ethyl or propyl;

(t1), (t2), (t3) and (t4) are independently zero or positive integers, preferably zero or a positive integer of from about 1 to about 10 (e.g., 1, 2, 3, 4, 5, 6); and

(a2) and (a3) are independently zero or 1.

The bifunctional L₁ linkers contemplated within the scope of the present invention include those in which combinations of substituents and variables are permissible so that such combinations result in stable compounds of Formula (I). For example, when (a3) is zero, Y₁₇ is not linked directly to Y₁₄.

For purposes of the present invention, when values for bifunctional linkers are positive integers equal to or greater than 2, the same or different bifunctional linkers can be employed.

R₂₁-R₂₈, in each occurrence, are independently the same or different when each of (t1), (t2), (t3) and (t4) is independently equal to or greater than 2.

In one embodiment, Y₁₄₋₁₅ and Y₁₇₋₁₉ are O or NH; and R₂₁₋₂₉ are independently hydrogen or methyl.

In another embodiment, Y₁₆ is O; Y₁₄₋₁₅ and Y₁₇₋₁₉ are O or NH; and R₂₁₋₂₉ are hydrogen.

In certain embodiments, L₁ is independently selected from among:

—(CH₂)ₜ₁-[C(═O)]ₐ₃—,

—(CH₂)ₜ₁Y₁₇—(CH₂)ₜ₂—(Y₁₈)ₐ₂-[C(═O)]ₐ₃—,

—(CH₂CH₂Y₁₇)ₜ₁-[C(═O)]ₐ₃—,

—(CH₂CH₂Y₁₇)ₜ₁(CH₂)ₜ₄—(Y₁₈)ₐ₂-[C(═O)]ₐ₃—,

—[CH₂CH₂)ₜ₂Y₁₇]ₜ₃(CH₂)ₜ₄—(Y₁₈)ₐ₂-[C(═O)]ₐ₃—,

—(CH₂)ₜ₁-[(CH₂)ₜ₂Y₁₇]ₜ₃(CH₂)ₜ₄—(Y₁₈)ₐ₂-[C(═O)]ₐ₃—,

—(CH₂)ₜ₁(Y₁₇)ₐ₂[C(═O)]ₐ₃(CH₂)ₜ₂—,

—(CH₂)ₜ₁(Y₁₇)ₐ₂[C(═O)]ₐ₃Y₁₄(CH₂)ₜ₂—,

—(CH₂)ₜ₁(Y₁₇)ₐ₂[C(═O)]ₐ₃(CH₂)ₜ₂—Y₁₅—(CH₂)ₜ₃—,

—(CH₂)ₜ₁(Y₁₇)ₐ₂[C(═O)]ₐ₃Y₁₄(CH₂)ₜ₂—Y₁₅—(CH₂)ₜ₃—,

—(CH₂)ₜ₁(Y₁₇)ₐ₂[C(═O)]ₐ₃(CH₂CH₂Y₁₉)ₜ₂(CH₂)ₜ₃—, and

—(CH₂)ₜ₁(Y₁₇)ₐ₂[C(═O)]ₐ₃Y₁₄(CH₂CH₂Y₁₉)ₜ₂(CH₂)ₜ₃—,

wherein

Y₁₄₋₁₅ and Y₁₇₋₁₉ are independently O, or NH;

(t1), (t2), (t3), and (t4) are independently zero or positive integers, preferably zero or positive integers of from about 1 to about 10 (e.g., 1, 2, 3, 4, 5, 6); and

(a2) and (a3) are independently zero or 1.

Y₁₇, in each occurrence, is the same or different, when (t1) or (t3) is equal to or greater than 2.

Y₁₉, in each occurrence, is the same or different, when (t2) is equal to or greater than 2.

In a further embodiment and/or alternative embodiments, illustrative examples of the L₁ group are selected from among:

—CH₂—-(CH₂)₂—, —(CH₂)₃—, —(CH₂)₄—, —(CH₂)₅—, —(CH₂)₆—, —NH(CH₂)—

—CH(NH₂)CH₂—,

—(CH₂)₄—C(═O)—, —(CH₂)₅—C(═O)—, —(CH₂)₆—C(═O)—,

—CH₂CH₂O—CH₂O—C(═O)—,

—(CH₂CH₂O)₂—CH₂O—C(═O)—,

—(CH₂CH₂O)₃—CH₂O—C(═O)—,

—(CH₂CH₂O)₂—C(═O)—,

—CH₂CH₂O—CH₂CH₂NH—C(═O)—,

—(CH₂CH₂O)₂—CH₂CH₂NH—C(═O)—,

—CH₂—O—CH₂CH₂O—CH₂CH₂NH—C(═O)—,

—CH₂—O—(CH₂CH₂O)₂—CH₂CH₂NH—C(═O)—,

—CH₂—O—CH₂CH₂O—CH₂C(═O)—,

—CH₂—O—(CH₂CH₂O)₂—CH₂C(═O)—,

—(CH₂)₄—C(═O)NH—, —(CH₂)₅—C(═O)NH—,

—(CH₂)₆—C(═O)NH—,

—CH₂CH₂O—CH₂O—C(═O)—NH—,

—(CH₂CH₂O)₂—CH₂O—C(═O)—NH—,

—(CH₂CH₂O)₃—CH₂O—C(═O)—NH—,

—(CH₂CH₂O)₂—C(═O)—NH—,

—CH₂CH₂O—CH₂CH₂NH—C(═O)—NH—,

—(CH₂CH₂O)₂—CH₂CH₂NH—C(═O)—NH—,

—CH₂—O—CH₂CH₂O—CH₂CH₂NH—C(O)—NH—,

—CH₂—O—(CH₂CH₂O)₂—CH₂CH₂NH—C(═O)—NH—,

—CH₂—O—CH₂CH₂O—CH₂C(═O)—NH—,

—CH₂—O—(CH₂CH₂O)₂—CH₂C(═O)—NH—,

—(CH₂CH₂O)₂—, —CH₂CH₂O—CH₂O—,

—(CH₂CH₂O)₂—CH₂CH₂NH—,

—(CH₂CH₂O)₃—CH₂CH₂NH—,

—CH₂CH₂O—CH₂CH₂NH—,

—(CH₂CH₂O)₂—CH₂CH₂NH—,

—CH₂—O—CH₂CH₂O—CH₂CH₂NH—,

—CH₂—O—(CH₂CH₂O)₂—CH₂CH₂NH—,

—CH₂—O—CH₂CH₂O—,

—CH₂—O—(CH₂CH₂O)₂—,

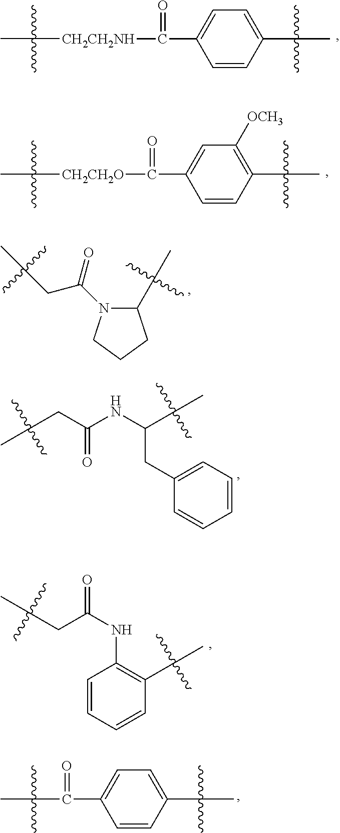

—C(═O)NH(CH₂)₂—, —CH₂C(═O)NH(CH₂)₂—,

—C(═O)NH(CH₂)₃—, —CH₂C(═O)NH(CH₂)₃—,

—C(═O)NH(CH₂)₄—, —CH₂C(═O)NH(CH₂)₄—,

—C(═O)NH(CH₂)₅—, —CH₂C(═O)NH(CH₂)₅—,

—C(═O)NH(CH₂)₆—, —CH₂C(═O)NH(CH₂)₆—,

—C(═O)O(CH₂)₂—, —CH₂C(═O)O(CH₂)₂—,

—C(═O)O(CH₂)₃—, —CH₂C(═O)O(CH₂)₃—,

—C(═O)O(CH₂)₄—, —CH₂C(═O)O(CH₂)₄—,

—C(═O)O(CH₂)₅—, —CH₂C(═O)O(CH₂)₅—,

—C(═O)O(CH₂)₆—, —CH₂C(═O)O(CH₂)₆—,

—(CH₂CH₂)₂NHC(═O)NH(CH₂)₂—,

—(CH₂CH₂)₂NHC(═O)NH(CH₂)₃—,

—(CH₂CH₂)₂NHC(═O)NH(CH₂)₄—,

—(CH₂CH₂)₂NHC(═O)NH(CH₂)₅—,

—(CH₂CH₂)₂NHC(═O)NH(CH₂)₆—,

—(CH₂CH₂)₂NHC(═O)O(CH₂)₂—,

—(CH₂CH₂)₂NHC(═O)O(CH₂)₃—,

—(CH₂CH₂)₂NHC(═O)O(CH₂)₄—,

—(CH₂CH₂)₂NHC(═O)O(CH₂)₅—,

—(CH₂CH₂)₂NHC(═O)O(CH₂)₆—,

—(CH₂CH₂)₂NHC(═O)(CH₂)₂—,

—(CH₂CH₂)₂NHC(═O)(CH₂)₃—,

—(CH₂CH₂)₂NHC(═O)(CH₂)₄—,

—(CH₂CH₂)₂NHC(═O)(CH₂)₅—, and

—(CH₂CH₂)₂NHC(═O)(CH₂)₆—.

In certain embodiments, L₂ is independently selected from among:

—(CR′₂₁R′₂₂)_{t′1}-[C(═Y′₁₆)]_{a′3}(CR′₂₇CR′₂₈)_{t′2}—,

—(CR′₂₁R′₂₂)_{t′1}Y′₁₄(CR′₂₃R′₂₄)_{t′2}-(Y′₁₅)_{a′2}-[C(═Y′₁₆)]_{a′3}(CR′₂₇CR′₂₈)_{t′3}—,

—(CR′₂₁R′₂₂CR′₂₃R′₂₄Y′₁₄)_{t′1}-[C(═Y′₁₆)]_{a′3}(CR′₂₇CR′₂₈)_{t′2}—,

—(CR′₂₁R′₂₂CR′₂₃R′₂₄Y′₁₄)_{t′1}(CR′₂₅R′₂₆)_{t′2}-(Y′₁₅)_{a′2}-[C(═Y′₁₆)]_{a′3}(CR′₂₇CR′₂₈)_{t′3}—,

—[(CR′₂₁R′₂₂CR′₂₃R′₂₄)_{t′2}Y′₁₄]_{t′1}(CR′₂₅R′₂₆)_{t′2}-(Y′₁₅)_{a′2}-[C(═Y′₁₆)]_{a′3}(CR′₂₇CR′₂₈)_{t′3}—,

—(CR′₂₁R′₂₂)_{t′1}-[(CR′₂₃R′₂₄)_{t′2}Y′₁₄]_{t′2}(CR′₂₅R′₂₆)_{t′3}-(Y′₁₅)_{a′2}-[C(═Y′₁₆)]_{a′3}(CR′₂₇CR′₂₈)_{t′4}—,

—(CR′₂₁R′₂₂)_{t′1}(Y′₁₄)_{a′2}[C(═Y′₁₆)]_{a′3}(CR′₂₃R′₂₄)_{t′2}—,

—(CR′₂₁R′₂₂)_{t′1}(Y′₁₄)_{a′2}[C(═Y′₁₆)]_{a′3}Y′₁₅(CR′₂₃R′₂₄)_{t′2}—,

—(CR′₂₁R′₂₂)_{t′1}(Y′₁₄)_{a′2}[C(═Y′₁₆)]_{a′3}(CR′₂₃R′₂₄)_{t′2}—Y′₁₅—(CR′₂₃R′₂₄)_{t′3}—,

—(CR′₂₁R′₂₂)_{t′1}(Y′₁₄)_{a′2}[C(═Y′₁₆)]_{a′3}Y′₁₄(CR′₂₃R′₂₄)_{t′2}—Y′₁₅—(CR′₂₃R′₂₄)_{t′3}—,

—(CR′₂₁R′₂₂)_{t′1}(Y′₁₄)_{a′2}[C(═Y′₁₆)]_{a′3}(CR′₂₃R′₂₄CR′₂₅R′₂₆Y′₁₅)_{t′2}(CR′₂₇CR′₂₈)_{t′3}—,

—(CR′₂₁R′₂₂)_{t′1}(Y′₁₄)_{a′2}[C(═Y′₁₆)]_{a′3}Y′₁₇(CR′₂₃R′₂₄CR′₂₅R′₂₆Y′₁₅)_{t′2}(CR′₂₇CR′₂₈)_{t′3}—, and

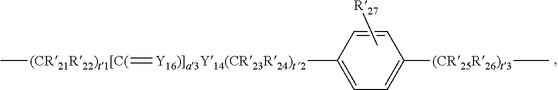

wherein:

Y′₁₆ is O, NR′₂₈, or S, preferably oxygen;

Y′₁₄₋₁₅ and Y′₁₇ are independently O, NR′₂₉, or S, preferably O, or NR′₂₉;

R′₂₁₋₂₇ are independently selected from among hydrogen, hydroxyl, amine, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy, preferably, hydrogen, methyl, ethyl or propyl;

R′₂₈₋₂₉ are independently selected from among hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy, preferably, hydrogen, methyl, ethyl or propyl;

(t′1), (t′2), (t′3) and (t′4) are independently zero or positive integers, preferably zero or a positive integer of from about 1 to about 10 (e.g., 1, 2, 3, 4, 5, 6); and

(a′2) and (a′3) are independently zero or 1.

The bifunctional L₂ linkers contemplated within the scope of the present invention include those in which combinations of variables and substituents of the linkers groups are permissible so that such combinations result in stable compounds of Formula (I). For example, when (a′3) is zero, Y′₁₄ is not linked directly to Y′₁₄ or Y′₁₇.

For purposes of the present invention, when values for bifunctional L₂ linkers including releasable linkers are positive integers equal to or greater than 2, the same or different bifunctional linkers can be employed.

In one embodiment, Y′₁₄₋₁₅ and Y′₁₇ are O or NH; and R′₂₁₋₂₉ are independently hydrogen or methyl.

In another embodiment, Y′₁₆ is O; Y′₁₄₋₁₅ and Y′₁₇ are O or NH; and R′₂₁₋₂₉ are hydrogen.

In certain embodiments, L₂ is selected from among:

—(CH₂)_{t′1}-[C(═O)]_{a′3}(CH₂)_{t′2}—,

—(CH₂)_{t′1}—Y′₁₄—(CH₂)_{t′2}-(Y′₁₅)_{a′2}-[C(═O)]_{a′3}(CH₂)_{t′3}—,

—(CH₂CH₂Y′₁₄)_{t′1}-[C(═O)]_{a′3}(CH₂)_{t′2}—,

—(CH₂CH₂Y′₁₄)_{t′1}(CH₂)_{t′2}-(Y′₁₅)_{a′2}-[C(═O)]_{a′3}(CH₂)_{t′3}—,

—[(CH₂CH₂)_{t′2}Y′₁₄]_{t′1}(CH₂)_{t′2}-(Y′₁₅)_{a′2}-[C(═O)]_{a′3}(CH₂)_{t′3}—,

—(CH₂)_{t′1}-[(CH₂)_{t′2}Y′₁₄]_{t′2}(CH₂)_{t′3}-(Y′₁₅)_{a′2}-[C(═O)]_{a′3}(CH₂)_{t′4}—,

—(CH₂)_{t′1}(Y′₁₄)_{a′2}[C(═O)]_{a′3}(CH₂)_{t′2}—,

—(CH₂)_{t′1}(Y′₁₄)_{a′2}[C(═O)]_{a′3}Y′₁₅(CH₂)_{t′2}—,

—(CH₂)_{t′1}(Y′₁₄)_{a′2}[C(═O)]_{a′3}(CH₂)_{t′2}—Y′₁₅—(CH₂)_{t′3}—,

—(CH₂)_{t′1}(Y′₁₄)_{a′2}[C(═O)]_{a′3}Y′₁₄(CH₂)_{t′2}—Y′₁₅—(CH₂)_{t′3}—,

—(CH₂)_{t′1}(Y′₁₄)_{a′2}[C(═O)]_{a′3}(CH₂CH₂Y′₁₅)_{t′2}(CH₂)_{t′3}—, and

—(CH₂)_{t′1}(Y′₁₄)_{a′2}[C(═O)]_{a′3}Y′₁₇(CH₂CH₂Y′₁₅)_{t′2}(CH₂)_{t′3}—,

wherein

Y′₁₄₋₁₅ and Y′₁₇ are independently O, or NH;

(t′1), (t′2), (t′3), and (t′4) are independently zero or positive integers, preferably 0 or positive integers of from about 1 to about 10 (e.g., 1, 2, 3, 4, 5, 6); and

(a′2) and (a′3) are independently zero or 1.

Y′₁₄, in each occurrence, is the same or different, when (t′1) or (t′2) is equal to or greater than 2.

Y′₁₅, in each occurrence, is the same or different, when (t′2) is equal to or greater than 2.

In a further embodiment and/or alternative embodiments, illustrative examples of the L₂ group are selected from among:

—CH₂— —(CH₂)₂—, —(CH₂)₃—, —(CH₂)₄—, —(CH₂)₅—, —(CH₂)₆—, —NH(CH₂)—

—CH(NH₂)CH₂—,

—O(CH₂)₂—, —C(═O)O(CH₂)₃—, —C(═O)NH(CH₂)₃—,

—C(═O)(CH₂)₂—, —C(═O)(CH₂)₃—,

—CH₂—C(═O)—O(CH₂)₃—,

—CH₂—C(═O)—NH(CH₂)₃—,

—CH₂—OC(═O)—O(CH₂)₃—,

—CH₂—OC(═O)—NH(CH₂)₃—,

—(CH₂)₂—C(═O)—O(CH₂)₃—,

—(CH₂)₂—C(═O)—NH(CH₂)₃—,

—CH₂C(═O)O(CH₂)₂—O—(CH₂)₂—,

—CH₂C(═O)NH(CH₂)₂—O—(CH₂)₂—,

—(CH₂)₂C(═O)O(CH₂)₂—O—(CH₂)₂—,

—(CH₂)₂C(═O)NH(CH₂)₂—O—(CH₂)₂—,

—CH₂C(═O)O(CH₂CH₂O)₂CH₂CH₂—,

—(CH₂)₂C(═O)O(CH₂CH₂O)₂CH₂CH₂—,

—(CH₂CH₂O)₂—, —CH₂CH₂O—CH₂O—.

—(CH₂CH₂O)₂—CH₂CH₂NH—, —(CH₂CH₂O)₃—CH₂CH₂NH—,

—CH₂CH₂O—CH₂CH₂NH—,

—CH₂—O—CH₂CH₂O—CH₂CH₂NH—,

—CH₂—O—(CH₂CH₂O)₂—CH₂CH₂NH—,

—CH₂—O—CH₂CH₂O—, —CH₂—O—(CH₂CH₂O)₂—,

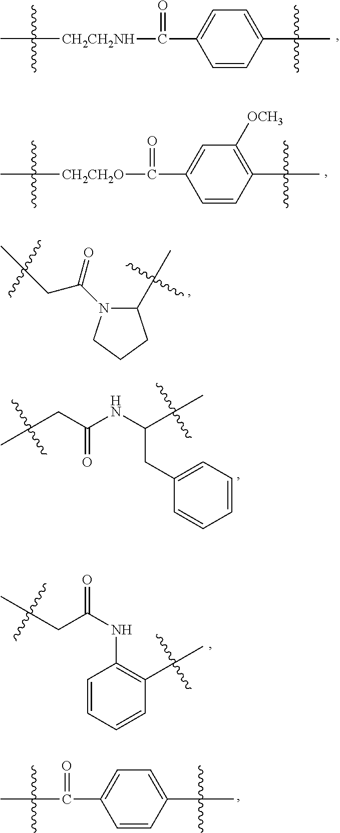

—(CH₂)₂NHC(═O)—(CH₂CH₂O)₂—,

—C(═O)NH(CH₂)₂—, —CH₂C(═O)NH(CH₂)₂—,

—C(═O)NH(CH₂)₃—, —CH₂C(═O)NH(CH₂)₃—,

—C(═O)NH(CH₂)₄—, —CH₂C(═O)NH(CH₂)₄—,

—C(═O)NH(CH₂)₅—, —CH₂C(═O)NH(CH₂)₅—,

—C(═O)NH(CH₂)₆—, —CH₂C(═O)NH(CH₂)₆—,

—C(═O)O(CH₂)₂—, —CH₂C(═O)O(CH₂)₂—,

—C(═O)O(CH₂)₃—, —CH₂C(═O)O(CH₂)₃—,

—C(═O)O(CH₂)₄—, —CH₂C(═O)O(CH₂)₄—,

—C(═O)O(CH₂)₅—, —CH₂C(═O)O(CH₂)₅—,

—C(═O)O(CH₂)₆—, —CH₂C(═O)O(CH₂)₆—,

—(CH₂CH₂)₂NHC(═O)NH(CH₂)₂—,

—(CH₂CH₂)₂NHC(═O)NH(CH₂)₃—,

—(CH₂CH₂)₂NHC(═O)NH(CH₂)₄—,

—(CH₂CH₂)₂NHC(═O)NH(CH₂)₅—,

—(CH₂CH₂)₂NHC(═O)NH(CH₂)₆—,

—(CH₂CH₂)₂NHC(═O)O(CH₂)₂—,

—(CH₂CH₂)₂NHC(═O)O(CH₂)₃—,

—(CH₂CH₂)₂NHC(═O)O(CH₂)₄—,

—(CH₂CH₂)₂NHC(═O)O(CH₂)₅—,

—(CH₂CH₂)₂NHC(═O)O(CH₂)₆—,

—(CH₂CH₂)₂NHC(═O)(CH₂)₂—,

—(CH₂CH₂)₂NHC(═O)(CH₂)₃—,

—(CH₂CH₂)₂NHC(═O)(CH₂)₄—,

—(CH₂CH₂)₂NHC(═O)(CH₂)₅—, and

—(CH₂CH₂)₂NHC(═O)(CH₂)₆—.

In a further embodiment, the bifunctional linkers L₁ and L₂ can be a spacer having a substituted saturated or unsaturated, branched or linear, C₃₋₅₀ alkyl (i.e., C₃₋₄₀ alkyl, C₃₋₂₀ alkyl, C₃₋₁₅ alkyl, C₃₋₁₀ alkyl, etc.), wherein optionally one or more carbons are replaced with NR₆, O, S or C(═Y), (preferably O or NH), but not exceeding 70% (i.e., less than 60%, 50%, 40%, 30%, 20%, 10%) of the carbons being replaced.

### 4. The Bifunctional Spacers: L₁₁, L₁₂ and L₁₃ Groups

According to the present invention, the bifunctional spacers L₁₁₋₁₃ are independently selected from among:

—(CR₃₁R₃₂)_{q1}—; and

—Y₂₆(CR₃₁R₃₂)_{q1}—,

wherein:

Y₂₆ is O, NR₃₃, or S, preferably oxygen or NR₃₃;

R₃₁₋₃₂ are independently selected from among hydrogen, hydroxyl, C₁₋₆ alkyls, C₃₋₁₂ branched alkyls, C₃₋₈ cycloalkyls, C₁₋₆ substituted alkyls, C₃₋₈ substituted cycloalkyls, C₁₋₆ heteroalkyls, substituted C₁₋₆ heteroalkyls, C₁₋₆ alkoxy, phenoxy and C₁₋₆ heteroalkoxy, preferably, hydrogen, methyl, ethyl or propyl;

R₃₃ is selected from among hydrogen, C₁₋₆ alkyls, C₃₋₁₂ branched alkyls, C₃₋₈ cycloalkyls, C₁₋₆ substituted alkyls, C₃₋₈ substituted cycloalkyls, C₁₋₆ heteroalkyls, substituted C₁₋₆ heteroalkyls, C₁₋₆ alkoxy, phenoxy and C₁₋₆ heteroalkoxy, preferably, hydrogen, methyl, ethyl or propyl; and

(q1) is zero or a positive integer, preferably zero or an integer of from about 1 to about 10 (e.g., 1, 2, 3, 4, 5, 6).

The bifunctional spacers contemplated within the scope of the present invention include those in which combinations of substituents and variables are permissible so that such combinations result in stable compounds of Formula (I).

R₃₁ and R₃₂, in each occurrence, are independently the same or different when (q1) is equal to or greater than 2.

In one preferred embodiment, R₃₁₋₃₃ are independently hydrogen or methyl.

In certain preferred embodiments, R₃₁₋₃₂ are hydrogen or methyl; and Y₃ is O or NH.

The C(R₃₁)(R₃₂) moiety is the same or different when (q1) is equal to or greater than 2.

In a further and/or alternative embodiments, L₁₁₋₁₃ are independently selected from among:

—CH₂—, —(CH₂)₂—, —(CH₂)₃—, —(CH₂)₄—, —(CH₂)₅—, —(CH₂)₆—,

—O(CH₂)₂—, —O(CH₂)₃—, —O(CH₂)₄—, —O(CH₂)₅—, —O(CH₂)₆—, CH(OH)—,

—(CH₂CH₂O)—CH₂CH₂—,

—(CH₂CH₂O)₂—CH₂CH₂—,

—C(═O)O(CH₂)₃—, —C(═O)NH(CH₂)₃—,

—C(═O)(CH₂)₂—, —C(═O)(CH₂)₃—,

—CH₂—C(═O)—O(CH₂)₃—,

—CH₂—C(═O)—NH(CH₂)₃—,

—CH₂—OC(═O)—O(CH₂)₃—,

—CH₂—OC(═O)—NH(CH₂)₃—,

—(CH₂)₂—C(═O)—O(CH₂)₃—,

—(CH₂)₂—C(═O)—NH(CH₂)₃—,

—CH₂C(═O)O(CH₂)₂—O—(CH₂)₂—,

—CH₂C(═O)NH(CH₂)₂—O—(CH₂)₂—,

—(CH₂)₂C(═O)O(CH₂)₂—O—(CH₂)₂—,

—(CH₂)₂C(═O)NH(CH₂)₂—O—(CH₂)₂—,

—CH₂C(═O)O(CH₂CH₂O)₂CH₂CH₂—, and

—(CH₂)₂C(═O)O(CH₂CH₂O)₂CH₂CH₂—.

### 5. The Q Group

According to the present invention, the Q group contains one or more substituted or unsubstituted, saturated or unsaturated C4-30-containing moieties. The Q group includes one or more C4-30 aliphatic saturated or unsaturated hydrocarbons.

The Q group is represented by Formula (Ia):

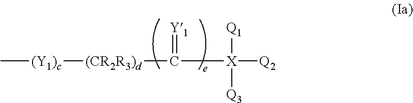

wherein

X is C, N or P;

Q₁ is H, C₁₋₃ alkyl, NR₅, OH, or

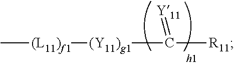

Q₂ is H, C₁₋₃ alkyl, NR₆, OH, or

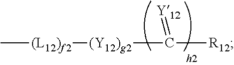

Q₃ is a lone electron pair, (═O), H, C₁₋₃ alkyl, NR₇, OH, or

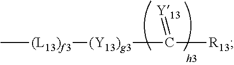

L₁₁, L₁₂ and L₁₃ are independently selected bifunctional spacers;

Y₁₁, Y₁₂, and Y₁₃ are independently O, S or NR₈, preferably oxygen or NH;

Y′₁₁, Y′₁₂, and Y′₁₃ are independently O, S or NR₈, preferably oxygen;

R₁₁, R₁₂ and R₁₃ are independently (substituted or unsubstituted) saturated or unsaturated C₄₋₃₀, and

all other variables are as defined above,

provided that Q includes at least one (one, two, three, preferably two) of R₁₁, R₁₂ and R₁₃.

In one preferred embodiment, R₁₁, R₁₂ and R₁₃ independently include a C4-30 saturated or unsaturated aliphatic hydrocarbon. More preferably, each aliphatic hydrocarbon is a saturated or unsaturated C8-24 hydrocarbon (yet more preferably, C12-22 hydrocarbon: C12-22 alkyl, C12-22 alkenyl, C12-22 alkoxy). Examples of aliphatic hydrocarbon include, but are not limited to, auroyl (C12), myristoyl (C14), palmitoyl (C16), stearoyl (C18), oleoyl (C18), and erucoyl (C22); saturated or unsaturated C12 alkyloxy, C14 alkyloxy, C16 alkyloxy, C18 alkyloxy, C20 alkyloxy, and C22 alkyloxy; and, saturated or unsaturated C12 alkyl, C14 alkyl, C16 alkyl, C18 alkyl, C20 alkyl, and C22 alkyl.

Preferably, at least two of R₁₁, R₁₂ and R₁₃ independently include a saturated or unsaturated C8-24 hydrocarbon (more preferably, C12-22 hydrocarbon).

Some examples of Q group are represented by the formula:

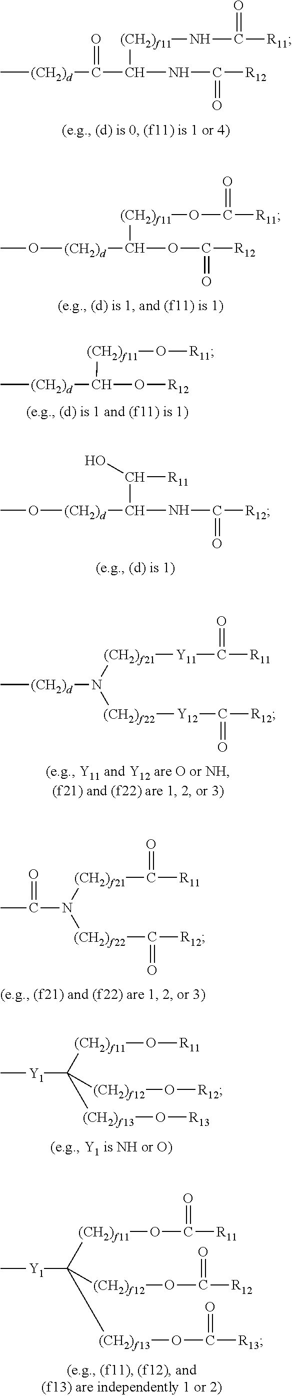

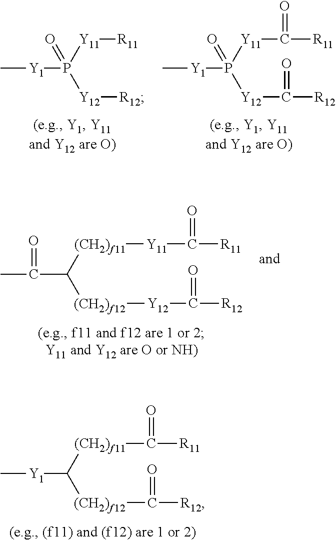

wherein,

Y₁ is O, S, or NR₃₁, preferably oxygen or NH;

R₁₁, R₁₂, and R₁₃ are independently substituted or unsubstituted, saturated or unsaturated. C₄₋₃₀ (alkyl, alkenyl, alkoxy);

R₃₁ is hydrogen, methyl or ethyl;

(d) is 0 or a positive integer, preferably 0 or an integer of from about 1 to about 10 (e.g., 1, 2, 3, 4, 5, 6);

(f11), (f12) and (f13) are independently 0, 1, 2, 3, or 4; and

(f21) and (f22) are independently 1, 2, 3 or 4.

In certain embodiments, the Q group includes diacylglycerol, diacylglycamide, dialkylpropyl, phosphatidylethanolamine or ceramide. Suitable diacylglycerol or diacylglycamide include a dialkylglycerol or dialkylglycamide group having alkyl chain length independently containing from about C₄ to about C₃₀, preferably from about C₈ to about C₂₄, saturated or unsaturated carbon atoms. The dialkylglycerol or dialkylglycamide group can further include one or more substituted alkyl groups.

The term “diacylglycerol” (DAG) used herein refers to a compound having two fatty acyl chains, R₁₁₁ and R₁₁₂. The R₁₁₁ and R₁₁₂ have the same or different about 4 to about 30 carbons (preferably about 8 to about 24) and are bonded to glycerol by ester linkages. The acyl groups can be saturated or unsaturated with various degrees of unsaturation. DAG has the general formula:

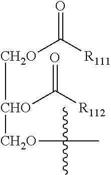

Examples of the DAG can be selected from among a dilaurylglycerol (C12), a dimyristylglycerol (C14, DMG), a dipalmitoylglycerol (C16, DPG), a distearylglycerol (C18, DSG), a dioleoylglycerol (C18), a dierucoyl (C22), a dilaurylglycamide (C12), a dimyristylglycamide (C14), a dipalmitoylglycamide (C16), a disterylglycamide (C18), a dioleoylglycamide (C18), dierucoylglyeamide (C22). Those of skill in the art will readily appreciate that other diacylglycerols are also contemplated.

The term “dialkyloxypropyl” refers to a compound having two alkyl chains, R₁₁₁ and R₁₁₂. The R₁₁₁ and R₁₁₂ alkyl groups include the same or different between about 4 to about 30 carbons (preferably about 8 to about 24). The alkyl groups can be saturated or have varying degrees of unsaturation. Dialkyloxypropyls have the general formula:

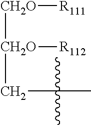

wherein R₁₁₁ and R₁₁₂ alkyl groups are the same or different alkyl groups having from about 4 to about 30 carbons (preferably about 8 to about 24). The alkyl groups can be saturated or unsaturated. Suitable alkyl groups include, but are not limited to, lauryl (C12), myristyl (C14), palmityl (C16), stearyl (C18), oleoyl (C18) and icosyl (C20).

In one embodiment, R₁₁₁ and R₁₁₂ are both the same, i.e., R₁₁₁ and R₁₁₂ are both myristyl (C14) or both oleoyl (C18), etc. In another embodiment, R₁₁₁ and R₁₁₂ are different, i.e., R₁₁₁ is myristyl (C14) and R₁₁₂ is stearyl (C18).

In another embodiment, the Q group can include phosphatidylethanolamines (PE). The phosphatidylethanolamines useful for the releasable fusogenic lipid conjugation can contain saturated or unsaturated fatty acids with carbon chain lengths in the range of about 4 to about 30 carbons (preferably about 8 to about 24). Suitable phosphatidylethanolamines include, but are not limited to: dimyristoylphosphatidylethanolamine (DMPE), dipalmitoylphosphatidylethanolamine (DPPE), dioleoylphosphatidylethanolamine (DOPE) and distearoylphosphatidylethanolamine (DSPE).

In yet another embodiment, the Q group can include ceramides (Cer). Ceramides have only one acyl group. Ceramides can have saturated or unsaturated fatty acids with carbon chain lengths in the range of about 4 to about 30 carbons (preferably about 8 to about 24).

One preferred embodiment includes:

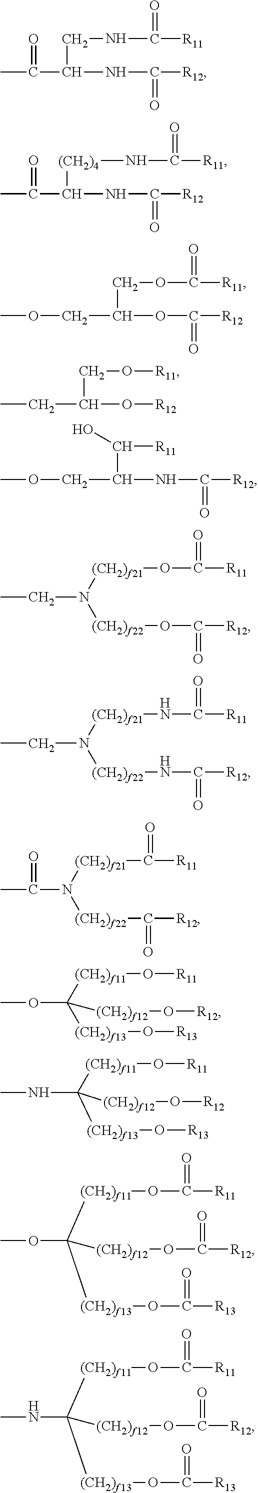

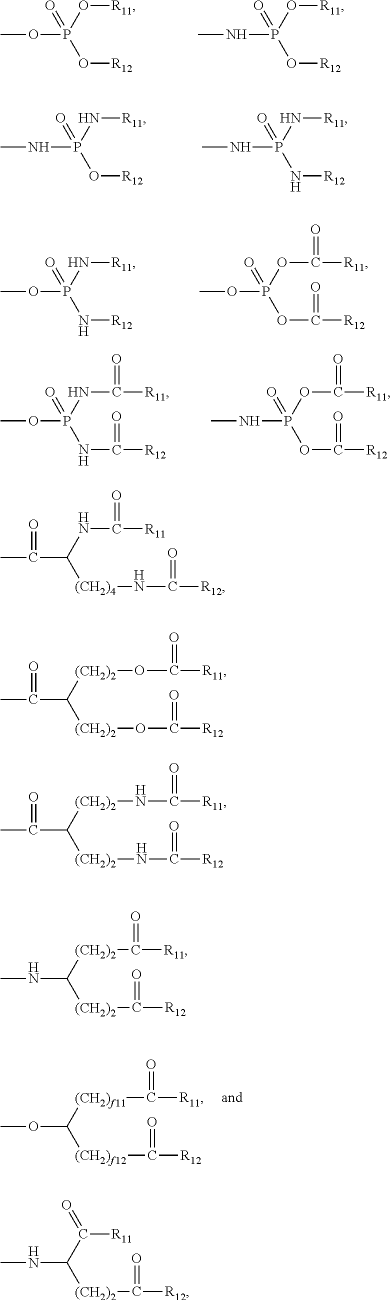

wherein R₁₁₋₁₃ are independently the same or different C12-22 saturated or unsaturated aliphatic hydrocarbons such as a dilauryl (C12), a dimyristyl (C14), a dipalmitoyl (C16), a distearyl (C18), a dioleoyl (C18), and a dierucoyl (C22);

(f11), (f12) and (f13) are independently 0, 1, 2, 3, or 4; and

(f21) and (f22) are independently 1, 2, 3 or 4.

### B. Preparation of Releasable Polymeric Lipids of Formula (I)

Synthesis of representative, specific compounds, is set forth in the Examples. Generally, however, the compounds of the present invention can be prepared in several fashions. In one embodiment, the methods of preparing compounds of Formula (I) described herein include reacting a polymer derivative having a ketal bond with a lipid derivative to provide a polymer-lipid conjugate having a ketal or acetal moiety. Alternatively, the methods include reacting a polymer derivative with a lipid derivative having a ketal or acetal moiety to provide a polymer-lipid conjugate.

In another embodiment, the methods of preparing compound of Formula (I) described herein include reacting an amine-containing compound with an aldehyde-containing compound to provide a polymer-lipid conjugate having an imine moiety. The amine can be a primary amine and the aldehyde can further contains aliphatic or aromatic substituents.

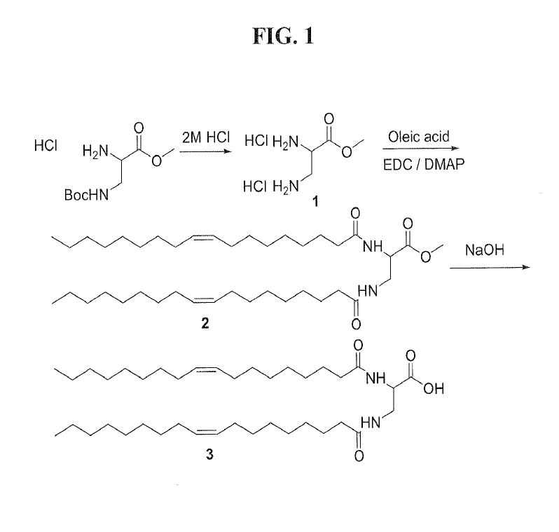

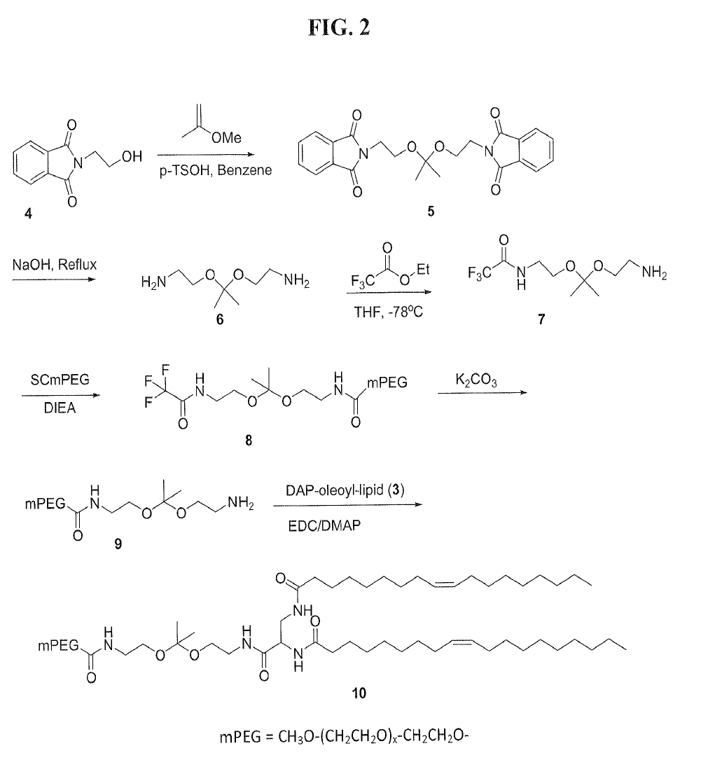

One representative example of preparing releasable polymeric lipids having a ketal-containing linker is shown in FIGS. 1 and 2. First, lipids are coupled with a nucleophilic multifunctional linker to provide compound 2 in the presence of a coupling agent such as EDC or DIPC. Preferably, the reaction is carried out in an inert solvent such as methylene chloride, chloroform, toluene, DMF or mixtures thereof. The reaction can be conducted in the presence of a base, such as DMAP, DIEA, pyridine, triethylamine, etc. at a temperature from −4° C. to about 70° C. (e.g. −4° C. to about 50° C.). In one preferred embodiment, the reaction is performed at a temperature from 0° C. to about 25° C. or 0° C. to about room temperature. Saponification of methyl ester of compound 2 provided a lipid derivative (compound 3).

A bifunctional linker containing a ketal bond (compound 6) is prepared. One of the diamines of the ketal-containing bifunctional linker is protected with ethyl trifluoroacetate. An activated polymer such as SCm PEG is reacted with the remaining nucleophile amin in the bifunctional linker, followed by removal of the trifluoroacetamide protecting group to provide a polymeric amine containing a ketal bond (compound 9). The polymeric amine is conjugated with a lipid derivative (compound 3) in the presence of a coupling agent to provide PEG lipids containing a ketal moiety.

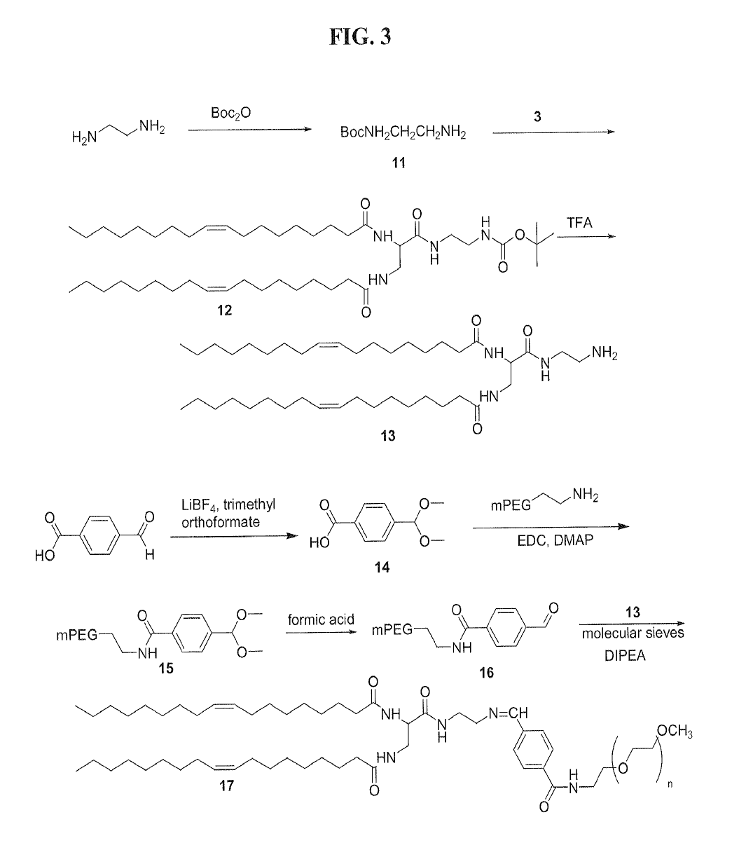

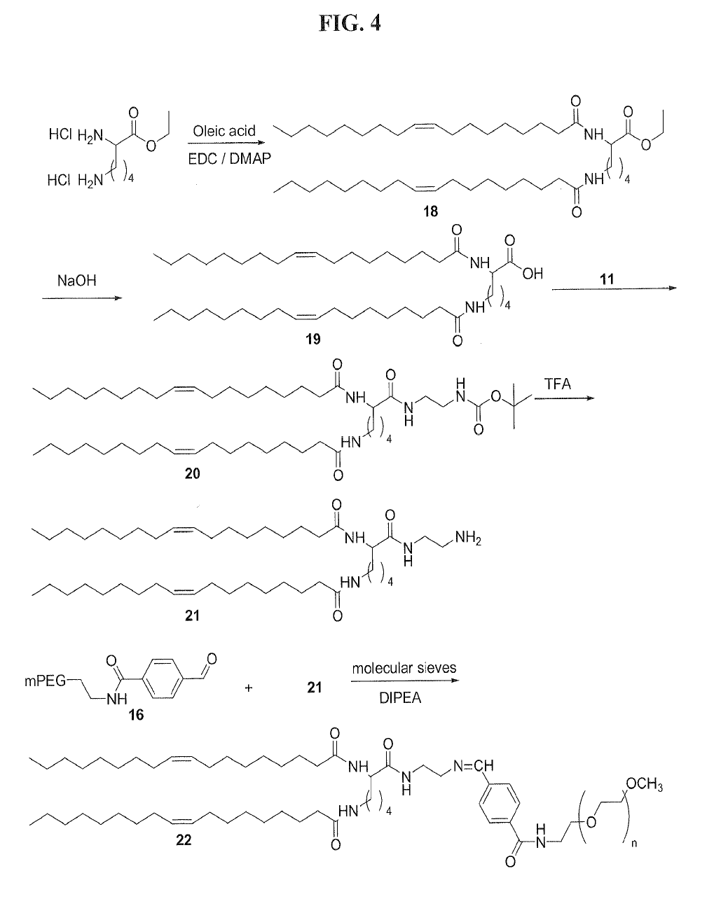

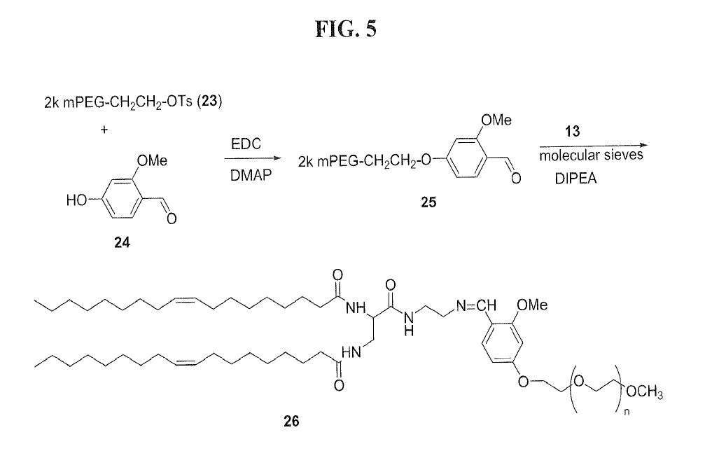

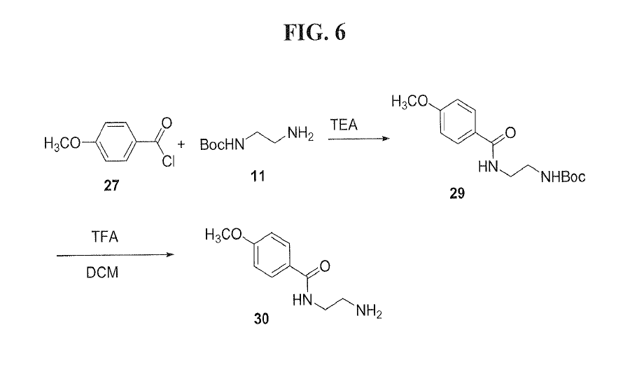

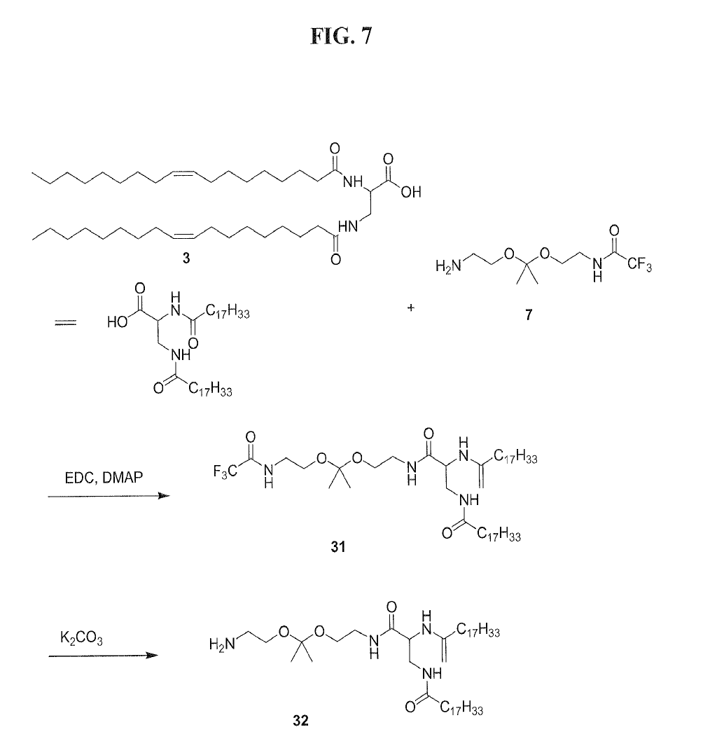

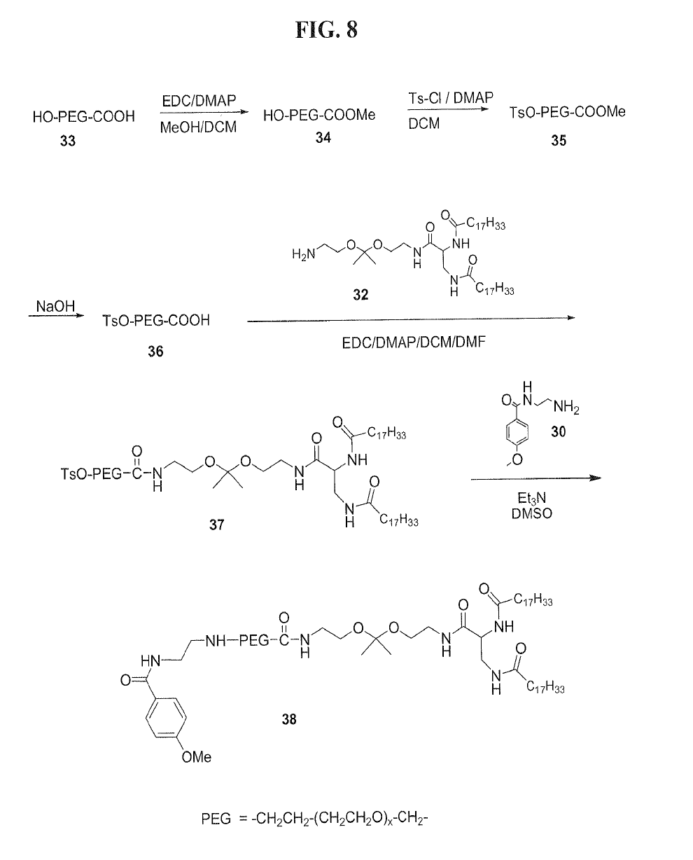

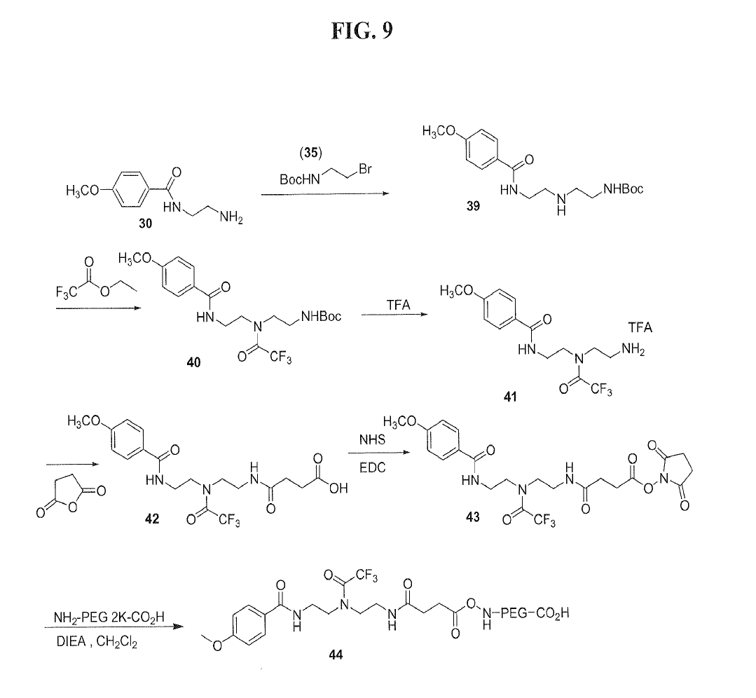

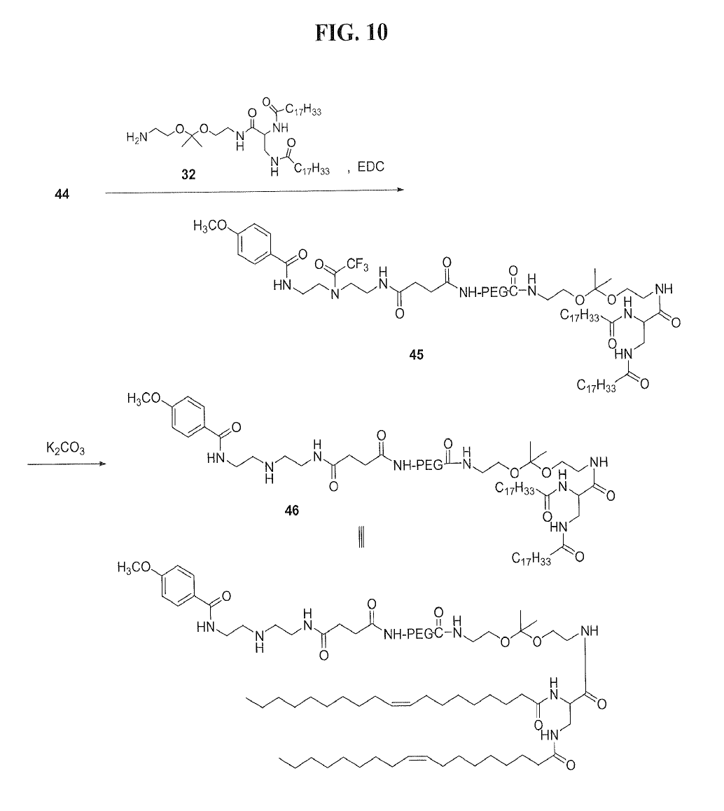

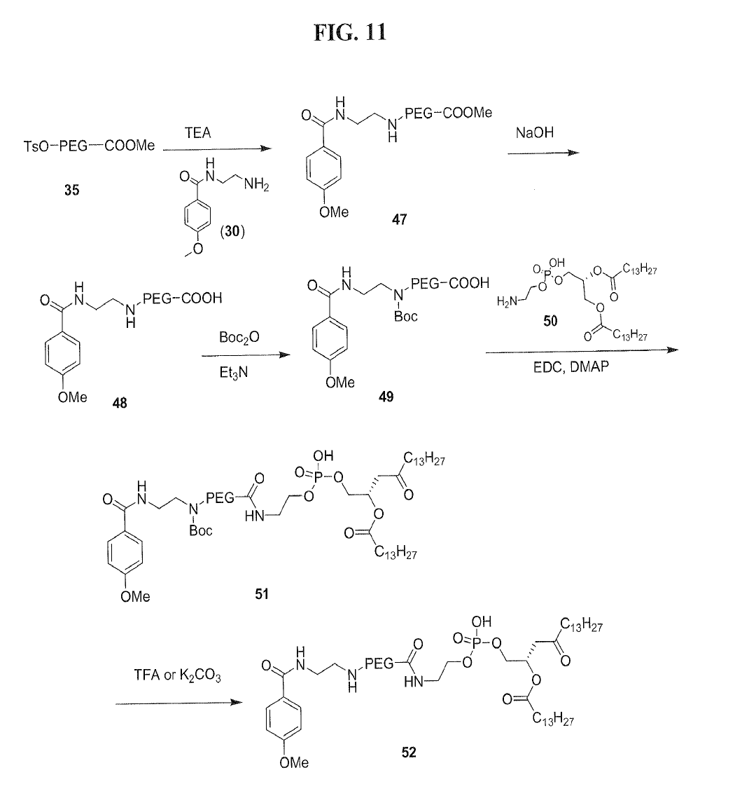

Another representative example of preparing polymer-lipid conjugates containing an imine moiety is shown in FIG. 3. A polymeric amine is reacted with a bifunctional linker to provide a polymer containing a protected aldehyde (compound 15). The aldehyde protecting group is removed to provide a polymeric aldehyde (compound 16). Lipids are coupled with a nucleophilic multifunctional linker containing an amine-protected group to provide a lipid derivative with an amine-protected group. After removal of the amine-protecting group, the lipid derivative having a terminal amine (compound 13) are reacted with the polymeric aldehyde to provide a polymeric lipid containing an imine bond.

Preferably, the reaction is carried out in an inert solvent such as methylene chloride, chloroform, toluene, DMF or mixtures thereof. The reaction is also preferably conducted in the presence of a base, such as DMAP, DIEA, pyridine, triethylamine, etc. at a temperature from −4° C. to about 70° C. (e.g. −4° C. to about 50° C.). In one preferred embodiment, the reaction is performed at a temperature from 0° C. to about 25° C. or 0° C. to about room temperature.

Conjugation of an amine to an acid or vice versa can be carried out using standard organic synthetic techniques in the presence of a base, using coupling agents known to those of ordinary skill in the art such as 1,3-diisopropylcarbodiimide (DIPC), dialkyl carbodiimides, 2-halo-1-alkylpyridinium halides, 1-(3-dimethylaminopropyl)-3-ethyl carbodiimide (EDC), propane phosphonic acid cyclic anhydride (PPACA) and phenyl dichlorophosphates.

In a further embodiment, an activated acid, such as NHS or PNP ester, can be used to react with a nucleophile in conjugation reaction, such as conjugation of compound 1 (amine, nucleophile) to compound 3 (acid, electrophile) or conjugation of compound 9 (amine, nucleophile) to compound 3 (acid, electrophile).

When an acid or electrophile is activated with a leaving group such as NHS, or PNP, a coupling agent is not required and the reaction proceeds in the presence of a base.

Removal of an amine-protecting group can be carried with a base such as NaOH or K₂CO₃. In one embodiment, deprotection of the trifluoroacetyl group is carried out with K₂CO₃. Alternatively, an amine-protecting group can be removed with a strong acid such as trifluoroacetic acid (TFA), HCl, sulfuric acid, etc., or catalytic hydrogenation, radical reaction, etc. Deprotection of Boc group is carried out with HCl solution in dioxane. The deprotection reaction can be carried out at a temperature from −4° C. to about 50° C. Preferably, the reaction is carried out at a temperature from 0° C. to about 25° C. or to room temperature. More preferably, the deprotection of Boc group is carried out at room temperature.

For example, compounds prepared by the methods described herein include:

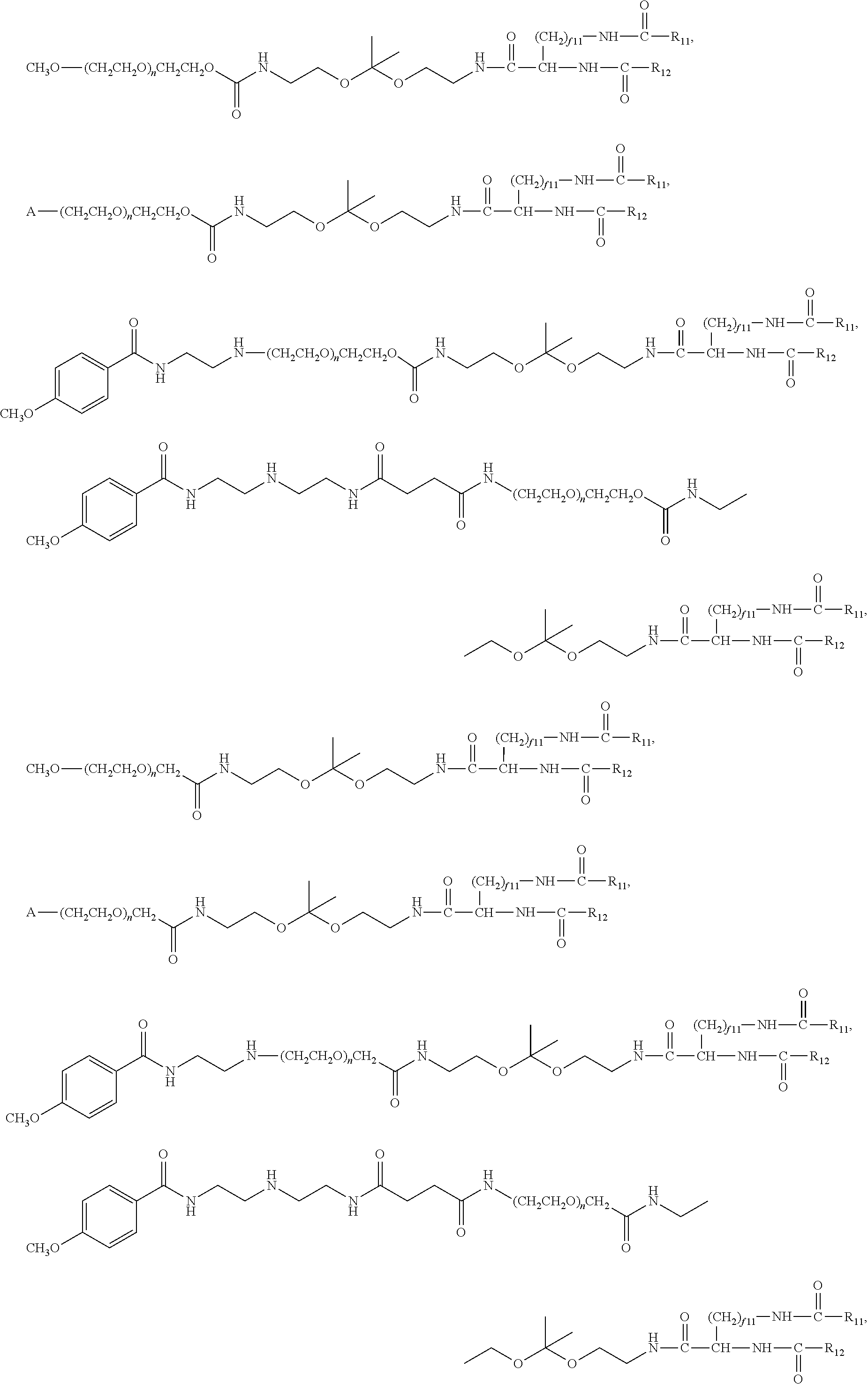

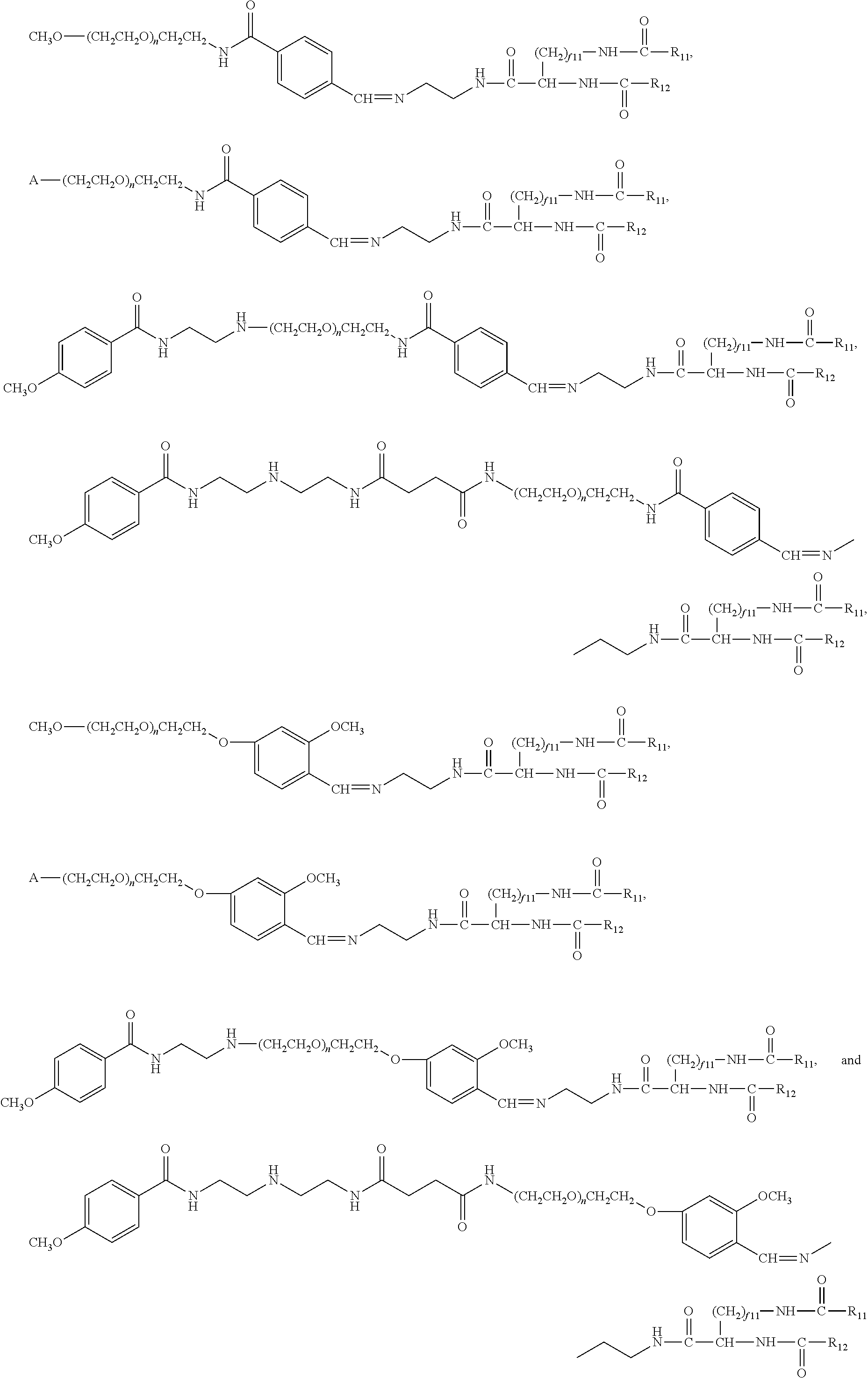

wherein

A is a targeting group;

(x) is the degree of polymerization so that the polymeric portion has the average molecular weight of from about 500 to about 5,000;

(f11) is zero, 1, 2, 3, or 4; and

R₁₁ and R₁₂ are independently C8-22 alkyl, C8-22 alkenyl, or C8-22 alkoxy.

Preferably, the releasable polymeric lipids of Formula (I) include:

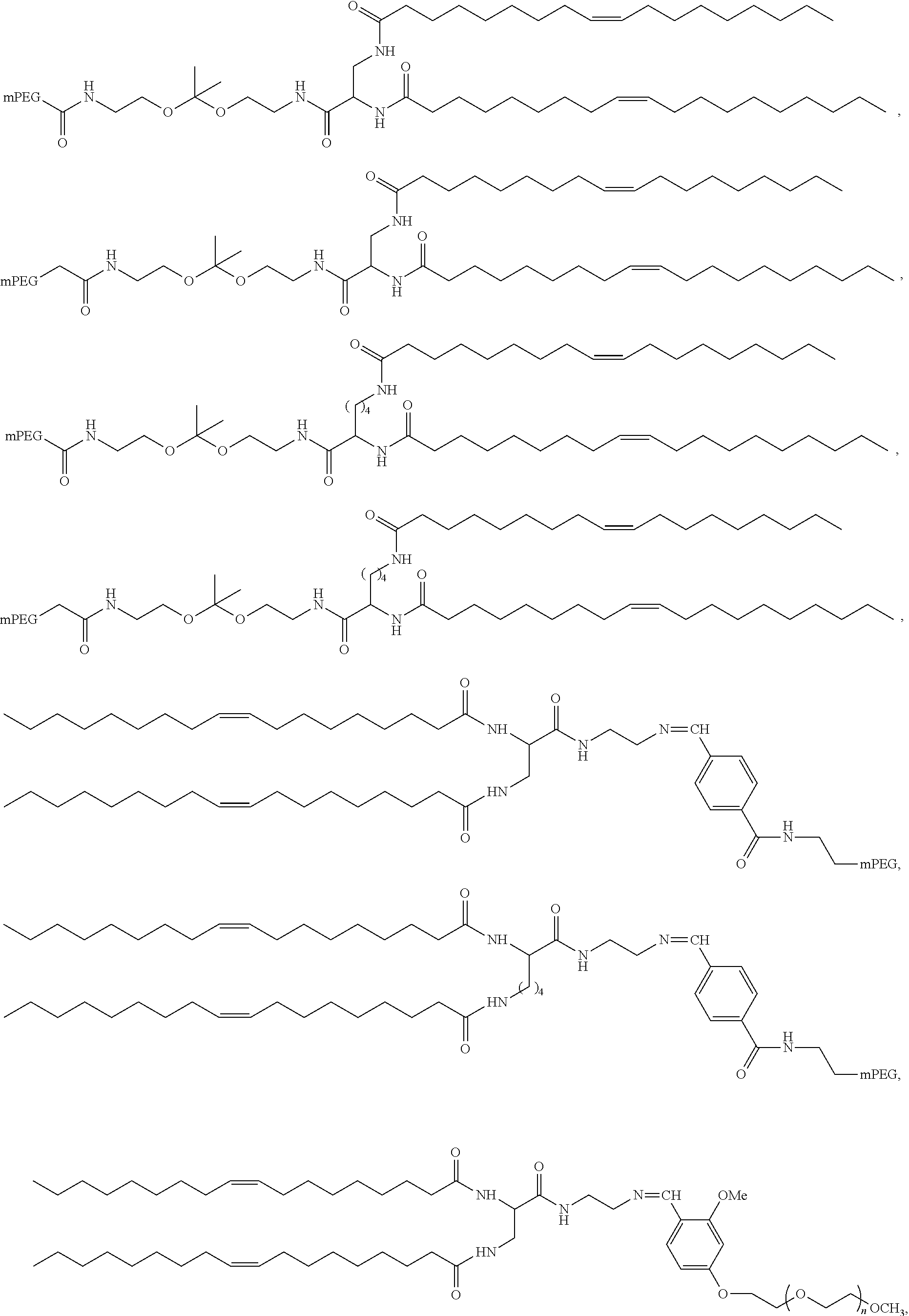

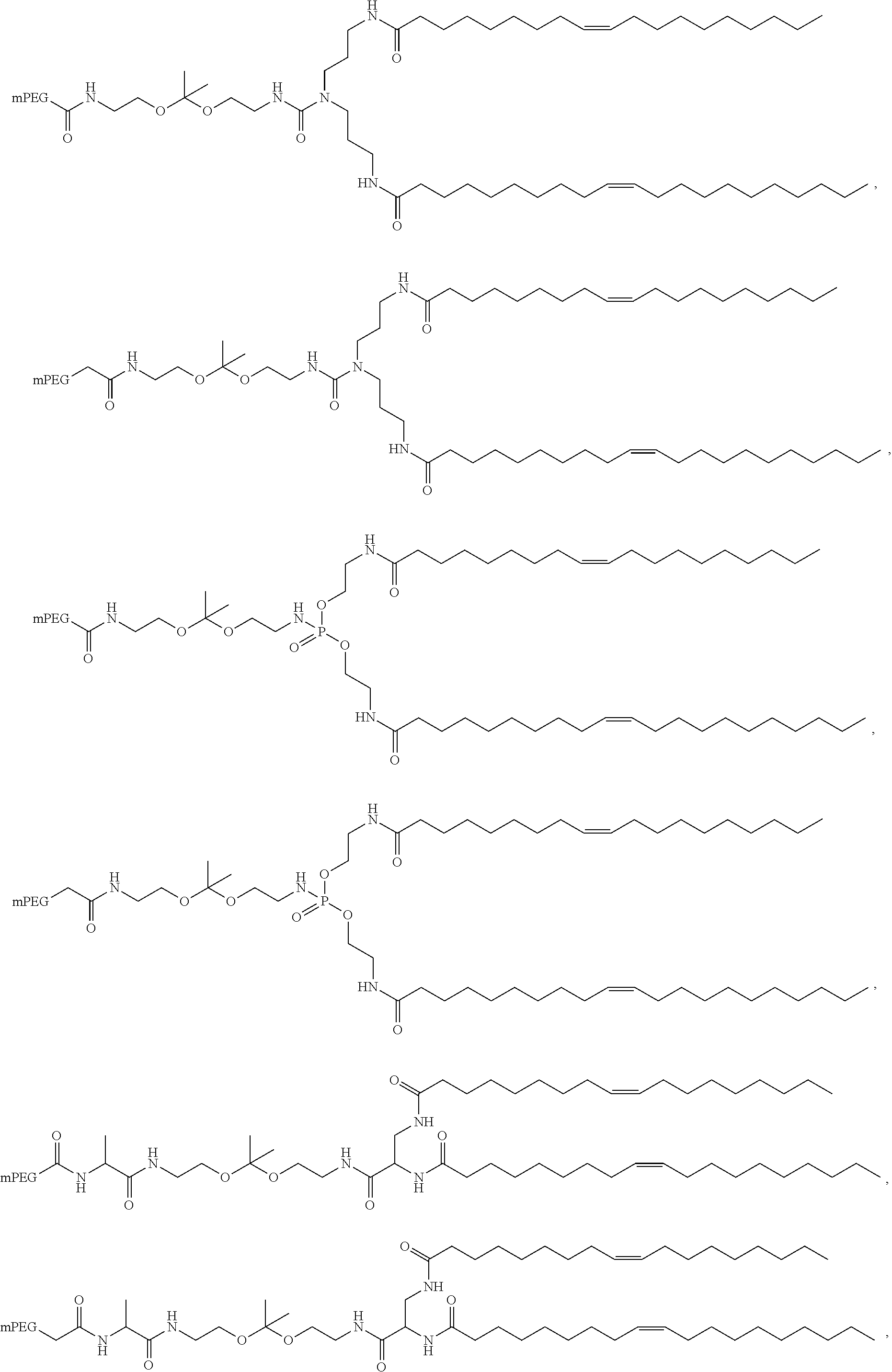

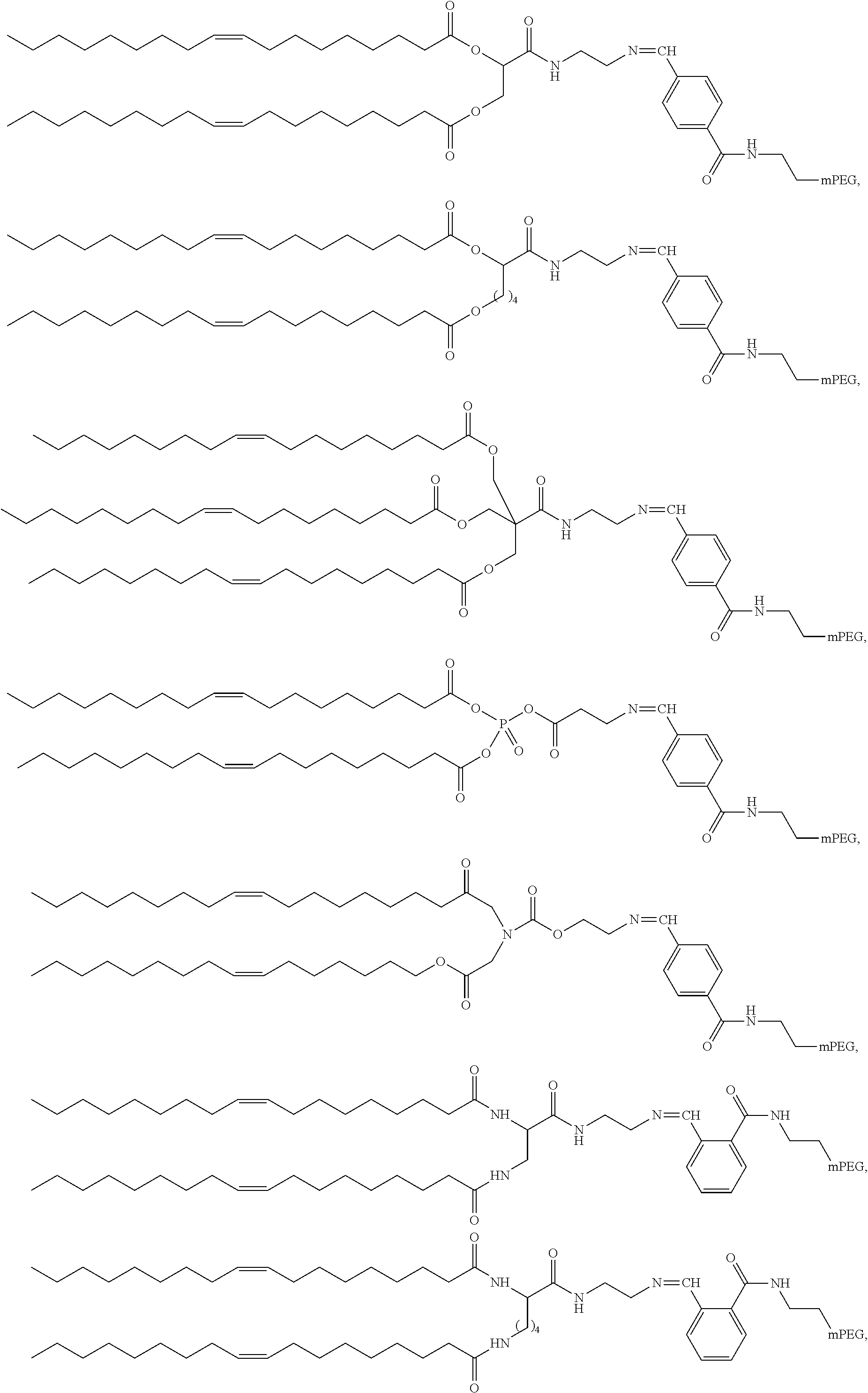

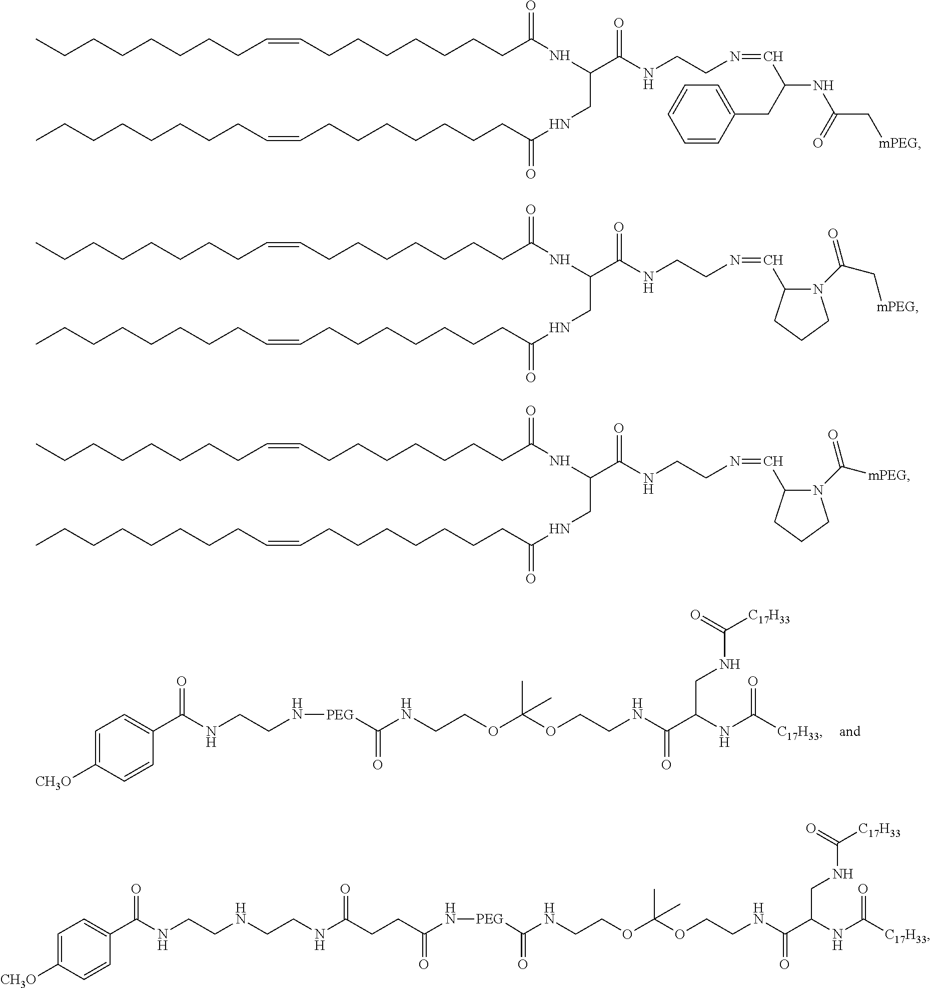

wherein

mPEG is CH₃—O—(CH₂CH₂O)ₙ—CH₂CH₂O—,

PEG is —(CH₂CH₂O)ₙ—CH₂— or —(CH₂CH₂O), —CH₂CH₂O—; and

(n) is an integer of from about 10 to about 460.

According to the present invention, releasable polymeric lipids useful in the preparation of nanoparticles include, but are not limited to:

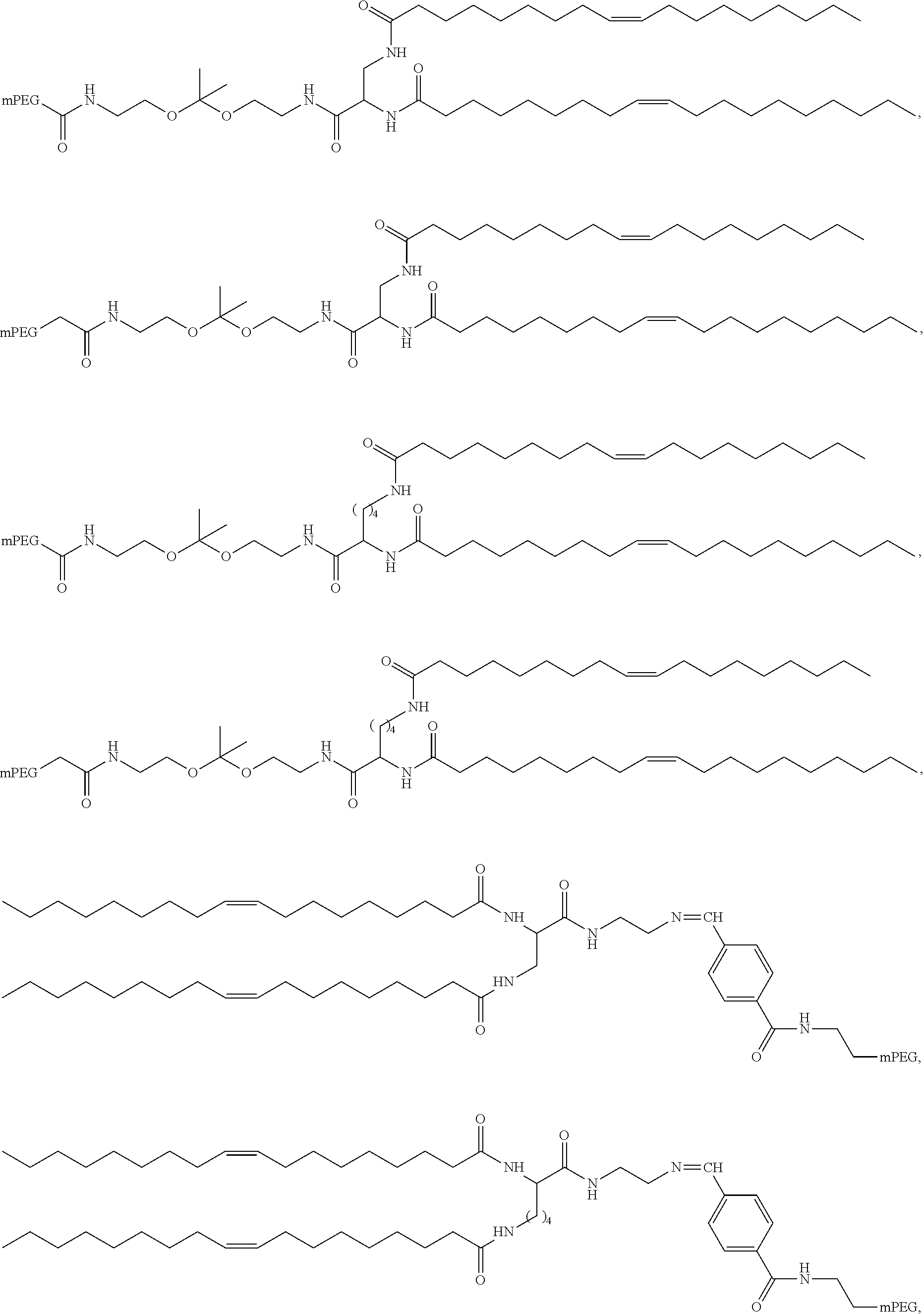

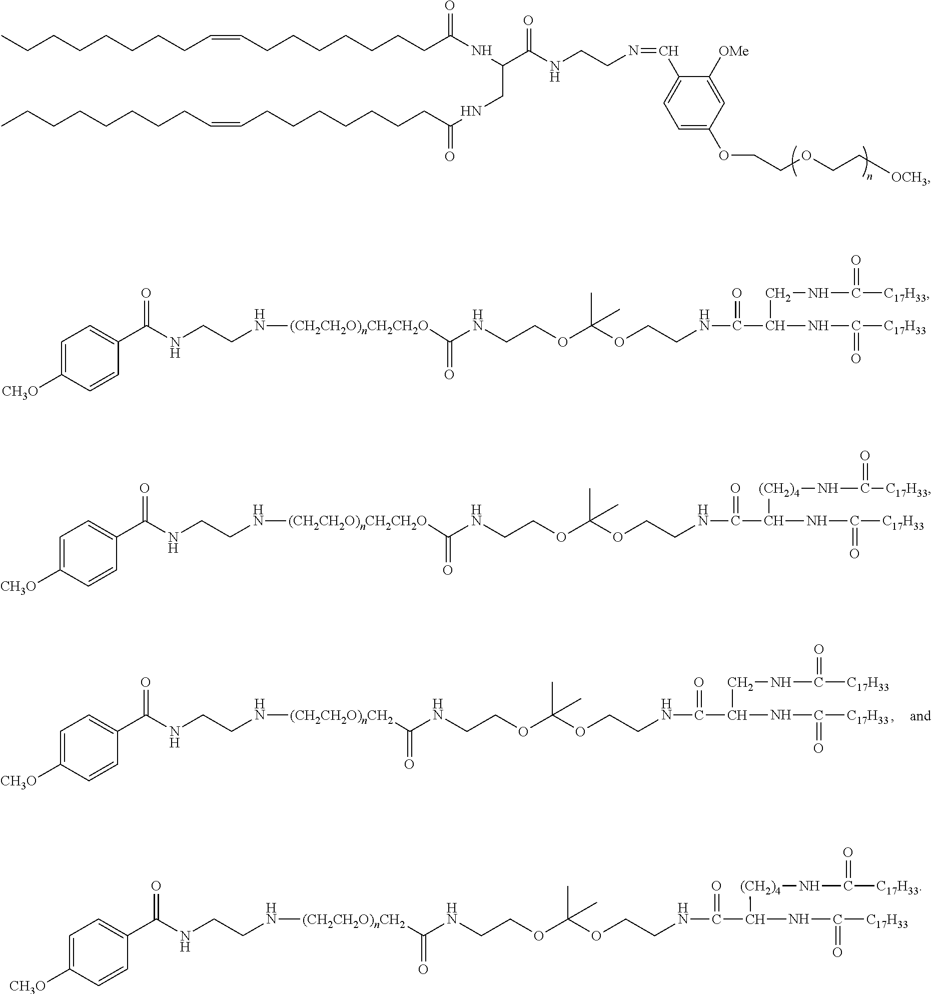

### C. Nanoparticle Compositions

### 1. Overview

According to the present invention, there are provided nanoparticle compositions containing a compound of Formula (I) for the delivery of nucleic acids.

In one aspect, the nanoparticle composition contains a releasable polymeric lipid of Formula (I), a cationic lipid, and a fusogenic lipid.

In one preferred aspect, the nanoparticle composition includes cholesterol.

In a further aspect of the present invention, the nanoparticle composition described herein may contain art-known PEG lipids. The nanoparticle composition containing a mixture of cationic lipids, a mixture of different fusogenic lipids (non-cationic lipids) and/or a mixture of different optional PEG lipids are also contemplated.

In another preferred aspect, the nanoparticle composition contains a cationic lipid in a molar ratio ranging from about 10% to about 99.9% of the total lipid present in the nanoparticle composition.

The cationic lipid component can range from about 2% to about 60%, from about 5% to about 50%, from about 10% to about 45%, from about 15% to about 25%, or from about 30% to about 40% of the total lipid present in the nanoparticle composition.

In one preferred embodiment, the cationic lipid is present in amounts from about 15 to about 25% (i.e., 15, 17, 18, 20 or 25%) of the total lipid present in the nanoparticle composition.

According to the present invention, the nanoparticle compositions contain the total fusogenic lipid, including cholesterol and/or noncholesterol-based fusogenic lipid, in a molar ratio of from about 20% to about 85%, from about 25% to about 85%, from about 60% to about 80% (e.g., 65, 75, 78, or 80%) of the total lipid present in the nanoparticle composition. In one embodiment, the total fusogenic/non-cationic lipid is about 80% of the total lipid present in the nanoparticle composition.

In certain embodiments, a noncholesterol-based fusogenic/non-cationic lipid is present in a molar ratio of from about 25 to about 78% (25, 35, 47, 60, or 78%), or from about 45 to about 78% of the total lipid present in the nanoparticle composition. In one embodiment, a noncholesterol-based fusogenic/non-cationic lipid is about 60% of the total lipid present in the nanoparticle composition.

In certain embodiments, the nanoparticle composition includes cholesterol in addition to non-cholesterol fusogenic lipid, in a molar ratio ranging from about 0% to about 60%, from about 10% to about 60%, or from about 20% to about 50% (e.g., 20, 30, 40 or 50%) of the total lipid present in the nanoparticle composition. In one embodiment, cholesterol is about 20% of the total lipid present in the nanoparticle composition.

In certain embodiments, the PEG-lipids including compounds of Formula (I) contained in the nanoparticle composition ranges in a molar ratio of from about 0.5% to about 20%, from about 1.5% to about 18% of the total lipid present in the nanoparticle composition. In one embodiment of the nanoparticle composition, the PEG lipid is included in a molar ratio of from about 2% to about 10% (e.g., 2, 3, 4, 5, 6, 7, 8, 9 or 10%) of the total lipid. For example, the total PEG lipid is about 2% of the total lipid present in the nanoparticle composition.

For purposes of the present invention, the amount of a releasable fusogenic lipid contained in the nanoparticle composition shall be understood to mean the amount of a releasable polymeric lipid described herein alone, or the sum of a releasable polymeric lipid of Formula (I) and any additional art-known polymeric lipids (either releasable or non-releasable) if present in the nanoparticle composition.

### 2. Polymeric Lipids: Releasable Polymeric Lipids of Formula (I) and Optional PEG Lipids

According to the present invention, the nanoparticle composition described herein contains a polymeric lipid. The polymeric lipids extend circulation of nanoparticles and prevent premature excretion of nanoparticles from the body. The polymeric lipids allow a reduction in the immune response in the body. The PEG lipids also enhance stability of nanoparticles.

In one preferred aspect, the nanoparticle composition described herein contains a releasable polymeric of Formula (I). Without being bound by any theory, the releasable polymeric lipids of Formula (I) facilitate nucleic acids encapsulated in the nanoparticle release from endosomes and the nanoparticle after the nanoparticle enters cells.

In a further aspect of the invention, the nanoparticle composition described herein may include additional art-known PEG lipids. Additional suitable PEG lipids useful in the nanoparticle composition include PEGylated form of fusogenic/noncationic lipids. The PEG lipids include, for example, PEG conjugated to diacylglycerol (PEG-DAG), PEG conjugated to dialkyloxypropyls (PEG-DAA), PEG conjugated to phospholipid such as PEG coupled to phosphatidylethanolamine (PEG-PE), PEG conjugated to ceramides (PEG-Cer), PEG conjugated to cholesterol derivatives (PEG-Chol) or mixtures thereof. See U.S. Pat. Nos. 5,885,613 and 5,820,873, and US Patent Publication No. 2006/051405, the contents of each of which are incorporated herein by reference.

In addition to the releasable polymeric lipids described herein, the nanoparticle composition described herein may include a polyethyleneglycol-diacylglycerol or polyethylene-diacylglycamide (PEG-DAG). Suitable polyethyleneglycol-diacylglycerol or polyethyleneglycol-diacylglycamide conjugates include a dialkylglycerol or dialkylglycamide group having alkyl chain length independently containing from about C₄ to about C₃₀ (preferably from about C₈ to about C₂₄) saturated or unsaturated carbon atoms. The dialkylglycerol or dialkylglycamide group can further include one or more substituted alkyl groups.

The term “diacylglycerol” (DAG) used herein refers to a compound having two fatty acyl chains, R₁₁₁ and R₁₁₂. The R₁₁₁ and R₁₁₂ have the same or different carbon chain in length of about 4 to about 30 carbons (preferably about 8 to about 24) and are bonded to glycerol by ester linkages. The acyl groups can be saturated or unsaturated with various degrees of unsaturation. DAG has the general formula:

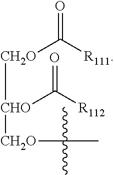

The DAG-PEG conjugate is a PEG-dilaurylglycerol (C12), a PEG-dimyristylglycerol (C14, DMG), a PEG-dipalmitoylglycerol (C16, DPG), a PEG-distearylglycerol (C18, DSG) or a PEG-dioleoylglycerol (C18). Those of skill in the art will readily appreciate that other diacylglycerols are also contemplated in the DAG-PEG. Suitable DAG-PEG conjugates for use in the present invention, and methods of making and using them, are described in U.S. Patent Publication No. 2003/0077829, and PCT Patent Application No. CA 02/00669, the contents of each of which are incorporated herein by reference.

Examples of the PEG-DAG conjugate can be selected from among PEG-dilaurylglycerol (C12), PEG-dimyristylglycerol (C14), PEG-dipalmitoylglycerol (C16), PEG-disterylglycerol (C18), PEG-dioleoylglycerol (C18), PEG-dilaurylglycamide (C12), PEG-dimyristylglycamide (C14), PEG-dipalmitoyl-glycamide (C16), PEG-disterylglycamide (C18), and PEG-dioleoylglycamide (C18).

In another embodiment, the polymeric nanoparticles described herein can includes a polyethyleneglycol-dialkyloxypropyl conjugates (PEG-DAG).

The term “dialkyloxypropyl” refers to a compound having two alkyl chains, R₁₁₁ and R₁₁₂. The R₁₁₁ and R₁₁₂ alkyl groups include the same or different carbon chain length between about 4 to about 30 carbons (preferably about 8 to about 24). The alkyl groups can be saturated or have varying degrees of unsaturation. Dialkyloxypropyls have the general formula:

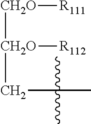

wherein R₁₁₁ and R₁₁₂ alkyl groups are the same or different alkyl groups having from about 4 to about 30 carbons (preferably about 8 to about 24). The alkyl groups can be saturated or unsaturated. Suitable alkyl groups include, but are not limited to, lauryl (C12), myristyl (C14), palmityl (C16), stearyl (C18), oleoyl (C18) and icosyl (C20).

In one embodiment, R₁₁₁ and R₁₁₂ are both the same, i.e., R₁₁₁ and R₁₁₂ are both myristyl (C14), both stearyl (C18) or both oleoyl (C18), etc. In another embodiment, R₁₁₁ and R₁₁₂ are different, i.e., R₁₁₁ is myristyl (C14) and R₁₁₂ is stearyl (C18). In one embodiment, the PEG-dialkylpropyl conjugates include the same R₁₁₁ and R₁₁₂.

In yet another embodiment, the nanoparticle composition described herein can include PEG conjugated to phosphatidylethanolamines (PEG-PE) in addition to the releasable polymeric lipids described herein. The phosphatidylethanolamines useful for the PEG lipid conjugation can contain saturated or unsaturated fatty acids with carbon chain lengths in the range of about 4 to about 30 carbons (preferably about 8 to about 24). Suitable phosphatidylethanolamines include, but are not limited to: dimyristoylphosphatidylethanolamine (DMPE), dipalmitoylphosphatidylethanolamine (DPPE), dioleoylphosphatidylethanolamine (DOPE) and distearoylphosphatidylethanolamine (DSPE).

In yet another embodiment, the nanoparticle composition described herein can include PEG conjugated to ceramides (PEG-Cer). Ceramides have only one acyl group. Ceramides can have saturated or unsaturated fatty acids with carbon chain lengths in the range of about 4 to about 30 carbons (preferably about 8 to about 24).

In alternative embodiments, the nanoparticle composition described herein can include PEG conjugated to cholesterol derivatives. The term “cholesterol derivative” means any cholesterol analog containing a cholesterol structure with modification, i.e., substitutions and/or deletions thereof. The term cholesterol derivative herein also includes steroid hormones and bile acids.

In one preferred aspect, the PEG is a polyethylene glycol with a number average molecular weight ranging from about 200 to about 20,000 daltons, more preferably from about 500 to about 10,000 daltons, yet more preferably from about 1,000 to about 5,000 daltons (i.e., about 1,500 to about 3,000 daltons). In one particular embodiment, the PEG has a molecular weight of about 2,000 daltons. In another particular embodiment, the PEG has a molecular weight of about 750 daltons.

Illustrative examples of PEG lipids includes N-(carbonyl-methoxypolyethyleneglycol)-1,2-dimyristoyl-sn-glycero-3-phosphoethanolamine (^{2 kDa}mPEG-DMPE or ^{5 kDa}mPEG-DMPE); N-(carbonyl-methoxypolyethyleneglycol)-1,2-dipalmitoyl-sn-glycero-3-phosphoethanolamine (^{2 kDa}mPEG-DPPE or ^{5 kDa}mPEG-DPPE); N-(carbonyl-methoxypolyethyleneglycol)-1,2-distearoyl-sn-glycero-3-phosphoethanolamine (^{750 Da}mPEG-DSPE 750, ^{2 kDa}mPEG-DSPE 2000, ^{5 kDa}EG-DSPE); pharmaceutically acceptable salts (i.e., sodium salt) and mixtures thereof.

In certain embodiments, the nanoparticle composition described herein can include a PEG lipid having PEG-DAG or PEG-ceramide, wherein PEG has an average molecular weight of from about 200 to about 20,000, preferably from about 500 to about 10,000, and more preferably from about 1,000 to about 5,000.

A few embodiments of PEG-DAG and PEG-ceramide are provided in Table 1.

TABLE 1
PEG-Lipid

| | |
|---|---|
| PEG-DAG | mPEG-diimyristoylglycerol |
| | mPEG-dipalmitoylglycerol |
| | mPEG-distearoylglycerol |
| PEG-Ceramide | mPEG-CerC8 |
| | mPEG-CerC14 |
| | mPEG-CerC16 |
| | mPEG-CerC20 |

The PEG lipid is selected from among PEG-DSPE, PEG-dipalmitoylglycamide (C16), PEG-Ceramide (C16), etc. and mixtures thereof. The structures of PEG-DSPE, PEG-dipalmitoylglycamide (C16), and PEG-Ceramide (C16) are as follows:

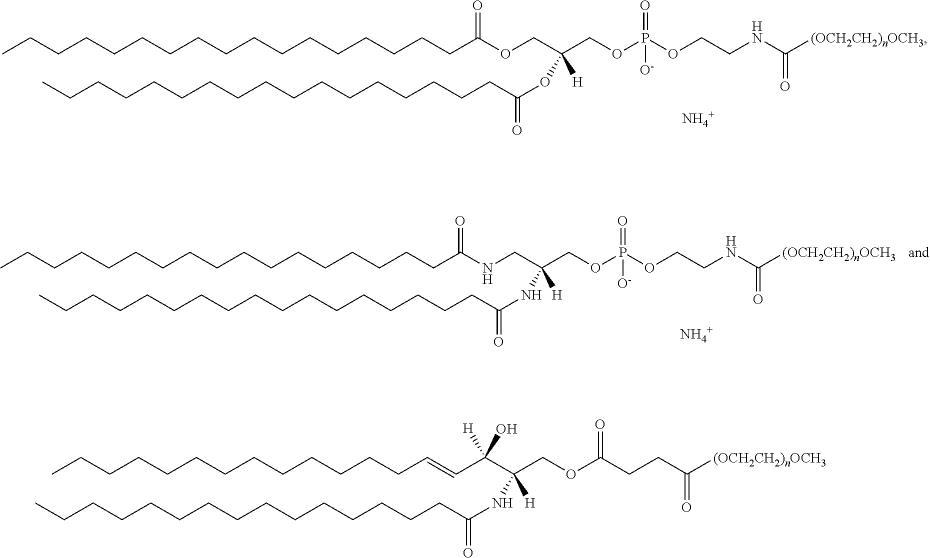

wherein, (n) is an integer from about 5 to about 2300, preferably from about 5 to about 460. In one embodiment, (n) is about 45.

### 3. Cationic Lipids

According to the present invention, the nanoparticle composition described herein can include a cationic lipid. Suitable lipids contemplated include, for example:
N-[1-(2,3-dioleoyloxy)propyl]-N,N,N-trimethylammonium chloride (DOTMA);
1,2-bis(oleoyloxy)-3-3-(trimethylammonium)propane or N-(2,3-dioleoyloxy)propyl)-N,N,N-trimethylammonium chloride (DOTAP);
1,2-bis(dimyrstoyloxy)-3-3-(trimethylammonia)propane (DMTAP);
1,2-dimyristyloxypropyl-3-dimethylhydroxyethylammonium bromide or N-(1,2-dimyristyloxyprop-3-yl)-N,N-dimethyl-N-hydroxyethyl ammonium bromide (DMRIE);
dimethyldioctadecylammonium bromide or N,N-distearyl-N,N-dimethylammonium bromide (DDAB);
3-(N—(N′,N′-dimethylaminoethane)carbamoyl)cholesterol (DC-Cholesterol);
3β-[N′,N′-diguanidinoethyl-aminoethane)carbamoyl cholesterol (BGTC);
2-(2-(3-(bis(3-aminopropyl)amino)propylamino)acetamido)-N,N-ditetradecylacetamide (RPR209120);
1,2-dialkenoyl-sn-glycero-3-ethylphosphocholines (i.e., 1,2-dioleoyl-sn-glycero-3-ethylphosphocholine, 1,2-distearoyl-sn-glycero-3-ethylphosphocholine and 1,2-dipalmitoyl-sn-glycero-3-ethylphosphocholine);
tetramethyltetrapalmitoyl spermine (TMTPS);
tetramethyltetraoleyl spermine (TMTOS);
tetramethlytetralauryl spermine (TMTLS);
tetramethyltetramyristyl spermine (TMTMS);
tetramethyldioleyl spermine (TMDOS);
2,5-bis(3-aminopropylamino)-N-(2-(dioctadecylamino)-2-oxoethyl) pentanamide (DOGS);
2,5-bis(3-aminopropylamino)-N-(2-(di(Z)-octadeca-9-dienylamino)-2-oxoethyl-1) pentanamide (DOGS-9-en);
2,5-bis(3-aminopropylamino)-N-(2-(di(9Z,12Z)-octadeca-9,12-dienylamino)-2-oxoethyl) pentanamide (DLinGS);
N4-Spermine cholesteryl carbamate (GL-67);
(9Z,9′Z)-2-(2,5-bis(3-aminopropylamino)pentanamido)propane-1,3-diyl-dioctadec-9-enoate (DOSPER);
2,3-dioleyloxy-N-[2(sperminecarboxamido)ethyl]-N,N-dimethyl-1-propanaminium trifluoroacetate (DOSPA);
1,2-dimyristoyl-3-trimethylammonium-propane; 1,2-distearoyl-3-trimethylammonium-propane;
dioctadecyldimethylammonium (DODMA);
distearyldimethylammonium (DSDMA);
N,N-dioleyl-N,N-dimethylammonium chloride (DODAC); pharmaceutically acceptable salts and mixtures thereof.

Details of cationic lipids are also described in US2007/0293449 and U.S. Pat. Nos. 4,897,355; 5,279,833; 6,733,777; 6,376,248; 5,736,392; 5,686,958; 5,334,761; 5,459,127; 2005/0064595; 5,208,036; 5,264,618; 5,279,833; 5,283,185; 5,753,613; and 5,785,992.

In one preferred aspect, the cationic lipids would carry a net positive charge at a selected pH, such as pH<13 (e.g. pH 6-12, pH 6-8). One preferred embodiment of the nanoparticle compositions includes the cationic lipids described herein having the structure:

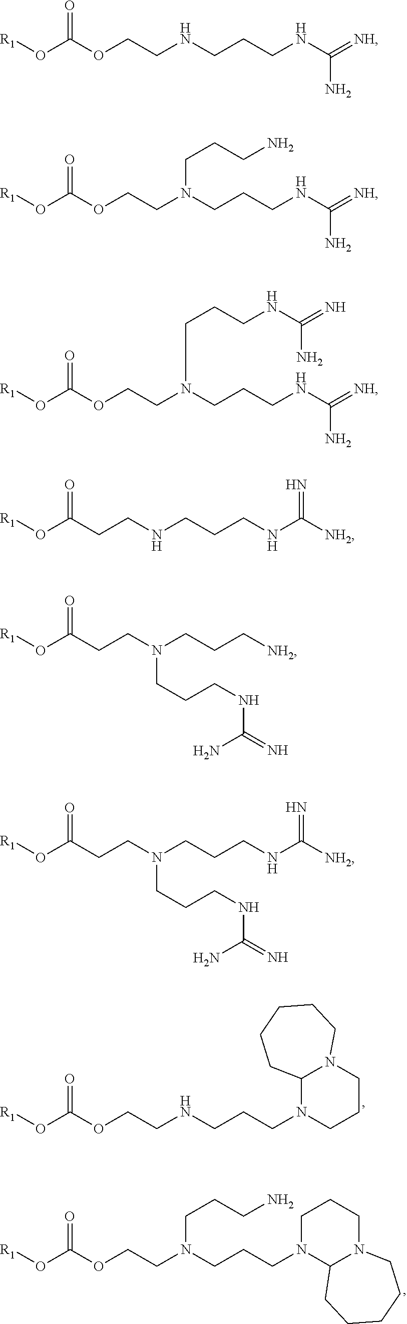

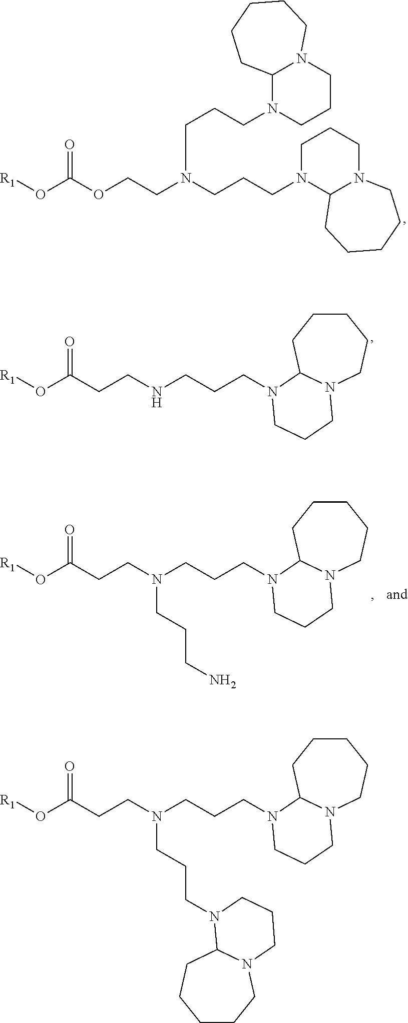

wherein R₁ is cholesterol or an analog thereof.

More preferably, a nanoparticle composition includes the cationic lipid having the structure:

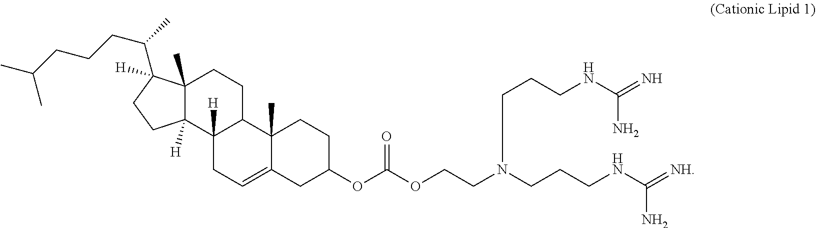

Details of cationic lipids are also described in PCT/US09/52396, the contents of which are incorporated herein by reference.

Additionally, commercially available preparations including cationic lipids can be used: for example, LIPOFECTIN® (cationic liposomes containing DOTMA and DOPE, from GIBCO/BRL, Grand Island, N.Y., USA); LIPOFECTAMINE® (cationic liposomes containing DOSPA and DOPE, from GIBCO/BRL, Grand Island, N.Y., USA); and TRANSFECTAM® (cationic liposomes containing DOGS from Promega Corp., Madison, Wis., USA).

### 4. Fusogenic/Non-Cationic Lipids

According to the present invention, the nanoparticle composition can contain a fusogenic lipid. The fusogenic lipids include non-cationic lipids such as neutral uncharged, zwitter ionic and anionic lipids. For purposes of the present invention, the terms “fusogenic lipid” and “non-cationic lipids” are interchangeable.

Neutral lipids include a lipid that exists either in an uncharged or neutral zwitter ionic form at a selected pH, preferably at physiological pH. Examples of such lipids include diacylphosphatidylcholine, diacylphosphatidylethanolamine, ceramide, sphingomyelin, cephalin, cholesterol, cerebrosides and diacylglycerols.

Anionic lipids include a lipid that is negatively charged at physiological pH. These lipids include, but are not limited to, phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and neutral lipids modified with other anionic modifying groups.

Many fusogenic lipids include amphipathic lipids generally having a hydrophobic moiety and a polar head group, and can form vesicles in aqueous solution.

Fusogenic lipids contemplated include naturally-occurring and synthetic phospholipids and related lipids.

A non-limiting list of the non-cationic lipids are selected from among phospholipids and nonphosphorous lipid-based materials, such as lecithin; lysolecithin; diacylphosphatidylcholine; lysophosphatidylcholine; phosphatidylethanolamine; lysophosphatidylethanolamine; phosphatidylserine; phosphatidylinositol; sphingomyelin; cephalin; ceramide; cardiolipin; phosphatidic acid; phosphatidylglycerol; cerebrosides; dicetylphosphate;
1,2-dilauroyl-sn-glycerol (DLG);
1,2-dimyristoyl-sn-glycerol (DMG);
1,2-dipalmitoyl-sn-glycerol (DPG);
1,2-distearoyl-sn-glycerol (DSG);
1,2-dilauroyl-sn-glycero-3-phosphatidic acid (DLPA);
1,2-dimyristoyl-sn-glycero-3-phosphatidic acid (DMPA);
1,2-dipalmitoyl-sn-glycero-3-phosphatidic acid (DPPA);
1,2-distearoyl-sn-glycero-3-phosphatidic acid (DSPA);
1,2-diarachidoyl-sn-glycero-3-phosphocholine (DAPC);
1,2-dilauroyl-sn-glycero-3-phosphocholine (DLPC);
1,2-dimyristoyl-sn-glycero-3-phosphocholine (DMPC);
1,2-dipalmitoyl-sn-glycero-3-ethylphosphocholine (DPePC);
1,2-dipalmitoyl-sn-glycero-3-phosphocholine or dipalmitoylphosphatidylcholine (DPPC);
1,2-distearoyl-sn-glycero-3-phosphocholine or distearoylphosphatidylcholine (DSPC);
1,2-dilauroyl-sn-glycero-3-phosphoethanolamine (DLPE);
1,2-dimyristoyl-sn-glycero-3-phosphoethanolamine or dimyristoylphosphoethanolamine (DMPE);
1,2-dipalmitoyl-sn-glycero-3-phosphoethanolamine or dipalmitoylphosphatidyl-ethanolamine (DPPE);
1,2-distearoyl-sn-glycero-3-phosphoethanolamine or distearoylphosphatidyl-ethanolamine (DSPE);
1,2-dioleoyl-sn-glycero-3-phosphoethanolamine or dioleoylphosphatidylethanolamine (DOPE);
1,2-dilauroyl-sn-glycero-3-phosphoglycerol (DLPG);
1,2-dimyristoyl-sn-glycero-3-phosphoglycerol (DMPG) or 1,2-dimyristoyl-sn-glycero-3-phospho-sn-1-glycerol (DMP-sn-1-G);
1,2-dipalmitoyl-sn-glycero-3-phosphoglycerol or dipalmitoylphosphatidylglycerol (DPPG);
1,2-distearoyl-sn-glycero-3-phosphoglycerol (DSPG) or 1,2-distearoyl-sn-glycero-3-phospho-sn-1-glycerol (DSP-sn-1-G);
1,2-dipalmitoyl-sn-glycero-3-phospho-L-serine (DPPS);
1-palmitoyl-2-linoleoyl-sn-glycero-3-phosphocholine (PLinoPC);
1-palmitoyl-2-oleoyl-sn-glycero-3-phosphocholine or palmitoyloleoylphosphatidylcholine (POPC);
1-palmitoyl-2-oleoyl-sn-glycero-3-phosphoglycerol (POPG);
1-palmitoyl-2-lyso-sn-glycero-3-phosphocholine (P-lyso-PC);
1-stearoyl-2-lyso-sn-glycero-3-phosphocholine (S-lyso-PC);
diphytanoylphosphatidylethanolamine (DPhPE);
1,2-dioleoyl-sn-glycero-3-phosphocholine or dioleoylphosphatidylcholine (DOPC);
1,2-diphytanoyl-sn-glycero-3-phosphocholine (DPhPC),
dioleoylphosphatidylglycerol (DOPG);
palmitoyloleoylphosphatidylethanolamine (POPE);
dioleoyl-phosphatidylethanolamine 4-(N-maleimidomethyl)-cyclohexane-1-carboxylate (DOPE-mal);
16-O-monomethyl PE;
16-O-dimethyl PE;
18-1-trans PE; 1-stearoyl-2-oleoyl-phosphatidyethanolamine (SOPE);
1,2-dielaidoyl-sn-glycero-3-phophoethanolamine (transDOPE); and pharmaceutically acceptable salts thereof and mixtures thereof. Details of the fusogenic lipids are described in US Patent Publication Nos. 2007/0293449 and 2006/0051405.

Noncationic lipids include sterols or steroid alcohols such as cholesterol.

Additional non-cationic lipids are, e.g., stearylamine, dodecylamine, hexadecylamine, acetylpalmitate, glycerolricinoleate, hexadecylstereate, isopropylmyristate, amphoteric acrylic polymers, triethanolaminelauryl sulfate, alkylarylsulfate polyethyloxylated fatty acid amides, and dioctadecyldimethyl ammonium bromide.

Anionic lipids contemplated include phosphatidylserine, phosphatidic acid, phosphatidylcholine, platelet-activation factor (PAF), phosphatidylethanolamine, phosphatidyl-DL-glycerol, phosphatidylinositol, phosphatidylinositol, cardiolipin, lysophosphatides, hydrogenated phospholipids, sphingoplipids, gangliosides, phytosphingosine, sphinganines, pharmaceutically acceptable salts and mixtures thereof.

Suitable noncationic lipids useful for the preparation of the nanoparticle composition described herein include diacylphosphatidylcholine (e.g., distearoylphosphatidylcholine, dioleoylphosphatidylcholine, dipalmitoylphosphatidylcholine and dilinoleoylphosphatidyl-choline), diacylphosphatidylethanolamine (e.g., dioleoylphosphatidylethanolamine and palmitoyloleoylphosphatidylethanolamine), ceramide or sphingomyelin. The acyl groups in these lipids are preferably fatty acids having saturated and unsaturated carbon chains such as linoyl, isostearyl, oleyl, elaidyl, petroselinyl, linolenyl, elaeostearyl, arachidyl, myristoyl, palmitoyl, and lauroyl. More preferably, the acyl groups are lauroyl, myristoyl, palmitoyl, stearoyl or oleoyl. Alternatively and/preferably, the fatty acids have saturated and unsaturated C₈-C₃₀ (preferably C₁₀-C₂₄) carbon chains.

A variety of phosphatidylcholines useful in the nanoparticle composition described herein includes:
1,2-didecanoyl-sn-glycero-3-phosphocholine (DDPC, C10:0, C10:0);
1,2-dilauroyl-sn-glycero-3-phosphocholine (DLPC, C12:0, C12:0);
1,2-dimyristoyl-sn-glycero-3-phosphocholine (DMPC, C14:0, C14:0);
1,2-dipalmitoyl-sn-glycero-3-phosphocholine (DPPC, C16:0, C16:0);
1,2-distearoyl-sn-glycero-3-phosphocholine (DSPC, C18:0, C18:0);
1,2-dioleoyl-sn-glycero-3-phosphocholine (DOPC, C18:1, C18:1);
1,2-dierucoyl-sn-glycero-3-phosphocholine (DEPC, C22:1, C22:1);
1,2-dieicosapentaenoyl-sn-glycero-3-phosphocholine (EPA-PC, C20:5, C20:5);
1,2-didocosahexaenoyl-sn-glycero-3-phosphocholine (DHA-PC, C22:6, C22:6);
1-myristoyl-2-palmitoyl-sn-glycero-3-phosphocholine (MPPC, C14:0, C16:0);
1-myristoyl-2-stearoyl-sn-glycero-3-phosphocholine (MSPC, C14:0, C18:0);
1-palmitoyl-2-stearoyl-sn-glycero-3-phosphocholine (PMPC, C16:0, C14:0);
1-palmitoyl-2-stearoyl-sn-glycero-3-phosphocholine (PSPC, C16:0, C18:0);
1-stearoyl-2-myristoyl-sn-glycero-3-phosphocholine (SMPC, C18:0, C14:0);
1-stearoyl-2-palmitoyl-sn-glycero-3-phosphocholine (SPPC, C18:0, C16:0);
1,2-myristoyl-oleoyl-sn-glycero-3-phosphoethanolamine (MOPC, C14:0, C18:0);
1,2-palmitoyl-oleoyl-sn-glycero-3-phosphoethanolamine (POPC, C16:0, C18:1);
1,2-stearoyl-oleoyl-sn-glycero-3-phosphoethanolamine (POPC, C18:0, C18:1), and pharmaceutically acceptable salts thereof and mixtures thereof.

A variety of lysophosphatidylcholine useful in the nanoparticle composition described herein includes:
1-myristoyl-2-lyso-sn-glycero-3-phosphocholine (M-LysoPC, C14:0);
1-malmitoyl-2-lyso-sn-glycero-3-phosphocholine (P-LysoPC, C16:0);
1-stearoyl-2-lyso-sn-glycero-3-phosphocholine (S-LysoPC, C18:0), and pharmaceutically acceptable salts thereof and mixtures thereof.

A variety of phosphatidylglycerols useful in the nanoparticle composition described herein are selected from among:
hydrogenated soybean phosphatidylglycerol (HSPG);
non-hydrogenated egg phosphatidylglycerol (EPG);
1,2-dimyristoyl-sn-glycero-3-phosphoglycerol (DMPG, C14:0, C14:0);
1,2-dipalmitoyl-sn-glycero-3-phosphoglycerol (DPPG, C16:0, C16:0);
1,2-distearoyl-sn-glycero-3-phosphoglycerol (DSPG, C18:0, C18:0);
1,2-dioleoyl-sn-glycero-3-phosphoglycerol (DOPG, C18:1, C18:1);
1,2-dierucoyl-sn-glycero-3-phosphoglycerol (DEPG, C22:1, C22:1);
1-palmitoyl-2-oleoyl-sn-glycero-3-phosphoglycerol (POPG, C16:0, C18:1), and pharmaceutically acceptable salts thereof and mixtures thereof.

A variety of phosphatidic acids useful in the nanoparticle composition described herein includes:
1,2-dimyristoyl-sn-glycero-3-phosphatidic acid (DMPA, C14:0, C14:0);
1,2-dipalmitoyl-sn-glycero-3-phosphatidic acid (DPPA, C16:0, C16:0);
1,2-distearoyl-sn-glycero-3-phosphatidic acid (DSPA, C18:0, C18:0), and pharmaceutically acceptable salts thereof and mixtures thereof.

A variety of phosphatidylethanolamines useful in the nanoparticle composition described herein includes:
hydrogenated soybean phosphatidylethanolamine (HSPE);
non-hydrogenated egg phosphatidylethanolamine (EPE);
1,2-dimyristoyl-sn-glycero-3-phosphoethanolamine (DMPE, C14:0, C14:0);
1,2-dipalmitoyl-sn-glycero-3-phosphoethanolamine (DPPE, C16:0, C16:0);
1,2-distearoyl-sn-glycero-3-phosphoethanolamine (DSPE, C18:0, C18:0);
1,2-dioleoyl-sn-glycero-3-phosphoethanolamine (DOPE, C18:1, C18:1);
1,2-dioleoyl-sn-glycero-3-phosphoethanolamine (DEPE, C22:1, C22:1);
1,2-dierucoyl-sn-glycero-3-phosphoethanolamine (POPE, C16:0, C18:1), and pharmaceutically acceptable salts thereof and mixtures thereof.

A variety of phosphatidylserines useful in the nanoparticle composition described herein includes:
1,2-dimyristoyl-sn-glycero-3-phospho-L-serine (DMPS, C14:0, C14:0);
1,2-dipalmitoyl-sn-glycero-3-phospho-L-serine (DPPS, C16:0, C16:0);
1,2-distearoyl-sn-glycero-3-phospho-L-serine (DSPS, C18:0, C18:0);
1,2-dioleoyl-sn-glycero-3-phospho-L-serine (DOPS, C18:1, C18:1);
1-palmitoyl-2-oleoyl-sn-3-phospho-L-serine (POPS, C16:0, C18:1), and pharmaceutically acceptable salts thereof and mixtures thereof.

In one preferred embodiment, suitable neutral lipids useful for the preparation of the nanoparticle composition described herein include, for example,

dioleoylphosphatidylethanolamine (DOPE),

distearoylphosphatidylethanolamine (DSPE),

palmitoyloleoylphosphatidylethanolamine (POPE),

egg phosphatidylcholine (EPC),

dipalmitoylphosphatidylcholine (DPPC),

distearoylphosphatidylcholine (DSPC),

dioleoylphosphatidylcholine (DOPC),

palmitoyloleoylphosphatidylcholine (POPC),

dipalmitoylphosphatidylglycerol (DPPG),

dioleoylphosphatidylglycerol (DOPG),

dioleoyl-phosphatidylethanolamine 4-(N-maleimidomethyl)-cyclohexane-1-carboxylate (DOPE-mal), cholesterol, pharmaceutically acceptable salts and mixtures thereof.

In certain preferred embodiments, the nanoparticle composition described herein includes DSPC, EPC, DOPE, etc, and mixtures thereof.

In a further aspect of the invention, the nanoparticle composition contains non-cationic lipids such as sterol. The nanoparticle composition preferably contains cholesterol or analogs thereof, and more preferably cholesterol.

In yet a further embodiment, the nanoparticle composition contains releasable fusogenic lipids based on an acid-labile imine linker and a zwitterion-containing moiety. Additional details of such releasable fusogenic lipids are described in U.S. Provisional Patent Application No. 61/115,378, and PCT Patent Application No. ______, filed on even date, and entitled “Releasable Fusogenic Lipids For Nucleic Acids Delivery Systems”, the contents of each of which are incorporated herein by reference.

### 5, Nucleic Acids/Oligonucleotides

The nanoparticle compositions described herein can be used for delivering various nucleic acids into cells or tissues. The nucleic acids include plasmids and oligonucleotides. Preferably, the nanoparticle compositions described herein are used for delivery of oligonucleotides.

In order to more fully appreciate the scope of the present invention, the following terms are defined. The artisan will appreciate that the terms, “nucleic acid” or “nucleotide” apply to deoxyribonucleic acid (“DNA”), ribonucleic acid, (“RNA”) whether single-stranded or double-stranded, unless otherwise specified, and to any chemical modifications or analogs thereof, such as, locked nucleic acids (LNA). The artisan will readily understand that by the term “nucleic acid,” included are polynucleic acids, derivates, modifications and analogs thereof. An “oligonucleotide” is generally a relatively short polynucleotide, e.g., ranging in size from about 2 to about 200 nucleotides, preferably from about 8 to about 50 nucleotides, more preferably from about 8 to about 30 nucleotides, and yet more preferably from about 8 to about 20 or from about 15 to about 28 in length. The oligonucleotides according to the invention are generally synthetic nucleic acids, and are single stranded, unless otherwise specified. The terms, “polynucleotide” and “polynucleic acid” may also be used synonymously herein.

The oligonucleotides (analogs) are not limited to a single species of oligonucleotide but, instead, are designed to work with a wide variety of such moieties, it being understood that linkers can attach to one or more of the 3′- or 5′-terminals, usually PO₄ or SO₄ groups of a nucleotide. The nucleic acid molecules contemplated can include a phosphorothioate internucleotide linkage modification, sugar modification, nucleic acid base modification and/or phosphate backbone modification. The oligonucleotides can contain natural phosphorodiester backbone or phosphorothioate backbone or any other modified backbone analogues such as LNA (Locked Nucleic Acid), PNA (nucleic acid with peptide backbone), CpG oligomers, and the like, such as those disclosed at Tides 2002, Oligonucleotide and Peptide Technology Conferences, May 6-8, 2002, Las Vegas, Nev. and Oligonucleotide & Peptide Technologies, 18th & 19th Nov. 2003, Hamburg, Germany, the contents of which are incorporated herein by reference.

Modifications to the oligonucleotides contemplated by the invention include, for example, the addition or substitution of functional moieties that incorporate additional charge, polarizability, hydrogen bonding, electrostatic interaction, and functionality to an oligonucleotide. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodouracil, backbone modifications, methylations, base-pairing combinations such as the isobases isocytidine and isoguanidine, and analogous combinations. Oligonucleotides contemplated within the scope of the present invention can also include 3′ and/or 5′ cap structure

For purposes of the present invention, “cap structure” shall be understood to mean chemical modifications, which have been incorporated at either terminus of the oligonucleotide. The cap can be present at the 5′-terminus (5′-cap) or at the 3′-terminus (3′-cap) or can be present on both termini. A non-limiting example of the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide; 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety. Details are described in WO 97/26270, the contents of which are incorporated by reference herein. The 3′-cap can include for example 4′,5′-methylene nucleotide; 1-(beta-D-erythrofuranosyl) nucleotide; 4′-thio nucleotide, carbocyclic nucleotide; 5¹-aminoalkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide; 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties. See also Beaucage and Iyer, 1993, Tetrahedron 49, 1925; the contents of which are incorporated by reference herein.

A non-limiting list of nucleoside analogs have the structure:

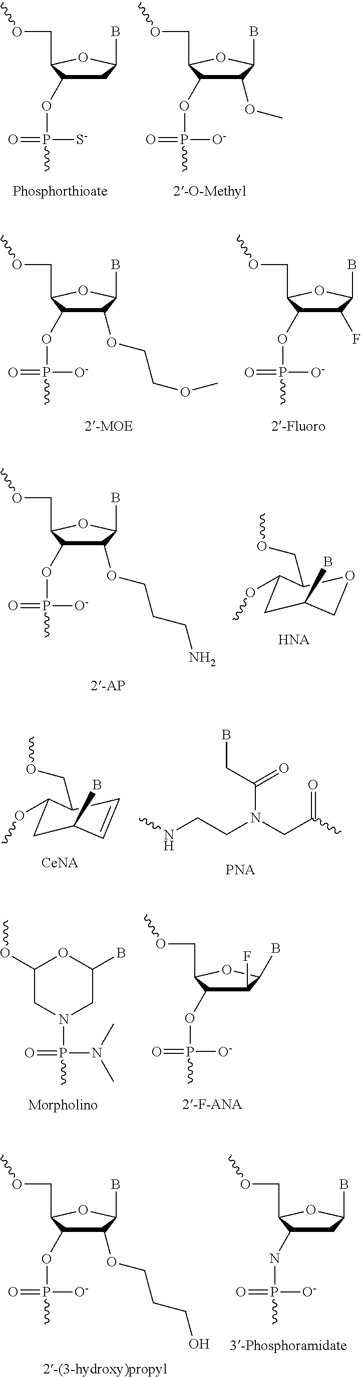

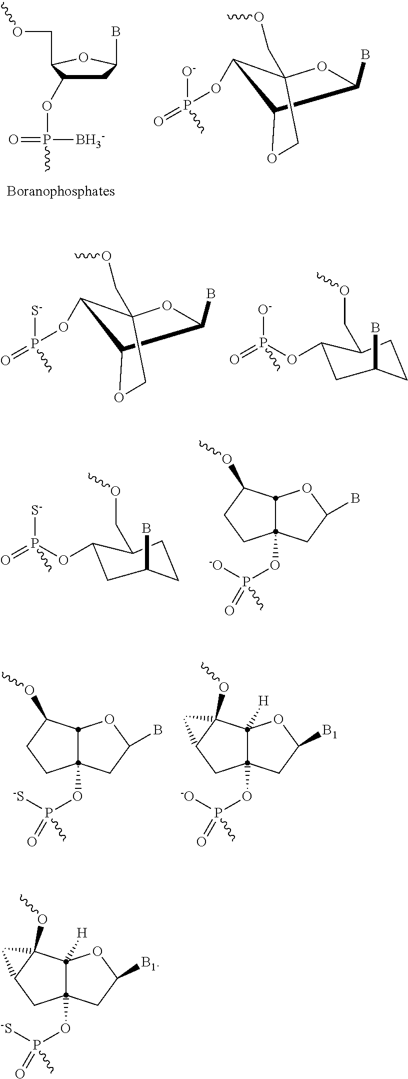

See more examples of nucleoside analogues described in Freier & Altmann; Nucl. Acid Res., 1997, 25, 4429-4443 and Uhlmann; Curr. Opinion in Drug Development, 2000, 3(2), 293-213, the contents of each of which are incorporated herein by reference.

The term “antisense,” as used herein, refers to nucleotide sequences which are complementary to a specific DNA or RNA sequence that encodes a gene product or that encodes a control sequence. The term “antisense strand” is used in reference to a nucleic acid strand that is complementary to the “sense” strand. In the normal operation of cellular metabolism, the sense strand of a DNA molecule is the strand that encodes polypeptides and/or other gene products. The sense strand serves as a template for synthesis of a messenger RNA (“mRNA”) transcript (an antisense strand) which, in turn, directs synthesis of any encoded gene product. Antisense nucleic acid molecules may be produced by any art-known methods, including synthesis. Once introduced into a cell, this transcribed strand combines with natural sequences produced by the cell to form duplexes. These duplexes then block either the further transcription of the mRNA or its translation. The designations “negative” or (−) are also art-known to refer to the antisense strand, and “positive” or (+) are also art-known to refer to the sense strand.

For purposes of the present invention, “complementary” shall be understood to mean that a nucleic acid sequence forms hydrogen bond(s) with another nucleic acid sequence. A percent complementarity indicates the percentage of contiguous residues in a nucleic acid molecule which can form hydrogen bonds, i.e., Watson-Crick base pairing, with a second nucleic acid sequence, i.e., 5, 6, 7, 8, 9, 10 out of 10 being 50%, 60%, 70%, 80%, 90%, and 100% complementary. “Perfectly complementary” means that all the contiguous residues of a nucleic acid sequence form hydrogen bonds with the same number of contiguous residues in a second nucleic acid sequence.

The nucleic acids (such as one or more same or different oligonucleotides or oligonucleotide derivatives) useful in the nanoparticle described herein can include from about 5 to about 1000 nucleic acids, and preferably relatively short polynucleotides, e.g., ranging in size preferably from about 8 to about 50 nucleotides in length (e.g., about 8, 9, 10, 11, 12, 13, 14, 15, 16, 17, 18, 19, 20, 21, 22, 23, 24, 25, 26, 27, 28, 29 or 30).

In one aspect of useful nucleic acids encapsulated within the nanoparticle described herein, oligonucleotides and oligodeoxynucleotides with natural phosphorodiester backbone or phosphorothioate backbone or any other modified backbone analogues include:

LNA (Locked Nucleic Acid);

PNA (nucleic acid with peptide backbone);

short interfering RNA (siRNA);

microRNA (miRNA);

nucleic acid with peptide backbone (PNA);

phosphorodiamidate morpholino oligonucleotides (PMO);

tricyclo-DNA;

decoy ODN (double stranded oligonucleotide);

catalytic RNA sequence (RNAi);

ribozymes;

aptamers;

spiegelmers (L-conformational oligonucleotides);

CpG oligomers, and the like, such as those disclosed at:

Tides 2002, Oligonucleotide and Peptide Technology Conferences, May 6-8, 2002, Las Vegas, Nev. and Oligonucleotide & Peptide Technologies, 18th & 19th Nov. 2003, Hamburg, Germany, the contents of which are incorporated herein by reference.

In another aspect of the nucleic acids encapsulated within the nanoparticle, oligonucleotides can optionally include any suitable art-known nucleotide analogs and derivatives, including those listed by Table 2, below:

TABLE 2
Representative Nucleotide Analogs And Derivatives

| | |
|---|---|
| 4-acetylcytidine | 5-methoxyaminomethyl-2-thiouridine |
| 5-(carboxyhydroxy- | beta, D-mannosylqueuosine |
| methyl)uridine | 5-methoxycarbonylmethyl-2- |
| 2′-O-methylcytidine | thiouridine |
| 5-methoxycarbonylmethyl- | 5-carboxymethylaminomethyl-2- |
| uridine | thiouridine |
| 5-methoxyuridine | 5-carboxymethylaminomethyluridine |
| Dihydrouridine | 2-methylthio-N6-isopentenyladenosine |
| 2′-O-methylpseudouridine | N-[(9-beta-D-ribofuranosyl-2-methyl- |
| D-galactosylqueuosine | thiopurine-6-yl)carbamoyl]threonine |
| 2′-O-methylguanosine | N-[(9-beta-D-ribofuranosylpurine-6- |
| 2′-halo-adenosine | yl)N-methylcarbamoyl]threonine |
| 2′-halo-guanosine | uridine-5-oxyacetic acid-methylester |
| 2′-halo-uridine | 2′-halo-cytidine |
| 2′-amino-adenosine | 2′-halo-thymine |
| 2′-amino-guanosine | 2′-halo-methylcytidine |
| 2′-amino-uridine | 2′-amino-cytidine |
| Inosine | 2′-amino-thymine |
| N6-isopentenyladenosine | 2′-amino-methylcytidine |
| 1-methyladenosine | uridine-5-oxyacetic acid |
| 1-methylpseudouridine | Wybutoxosine |
| 1-methylguanosine | Pseudouridine |
| 1-methylinosine | Queuosine |
| 2,2-dimethylguanosine | 2-thiocytidine |
| 2-methyladenosine | 5-methyl-2-thiouridine |
| 2-methylguanosine | 2-thiouridine |
| 3-methylcytidine | 4-thiouridine |
| 5-methylcytidine | 5-methyluridine |
| N6-methyladenosine | N-[(9-beta-D-ribofuranosylpurine-6-yl)- |
| 7-methylguanosine | carbamoyl]threonine |
| 5-methylaminomethyluridine | 2′-O-methyl-5-methyluridine |
| Locked-adenosine | 2′-O-methyluridine |
| Locked-guanosine | Wybutosine |
| Locked-uridine | 3-(3-amino-3-carboxy-propyl)uridine |
| | Locked-cytidine |
| | Locked-thymine |
| | Locked-methylcytidine |

In one preferred aspect, the target oligonucleotides encapsulated in the nanoparticles include, for example, but are not limited to, oncogenes, pro-angiogenesis pathway genes, pro-cell proliferation pathway genes, viral infectious agent genes, and pro-inflammatory pathway genes,

In one preferred embodiment, the oligonucleotide encapsulated within the nanoparticle described herein is involved in targeting tumor cells or downregulating a gene or protein expression associated with tumor cells and/or the resistance of tumor cells to anticancer therapeutics. For example, antisense oligonucleotides for downregulating any art-known cellular proteins associated with cancer, e.g., BCL-2 can be used for the present invention. See U.S. patent application Ser. No. 10/822,205 filed Apr. 9, 2004, the contents of which are incorporated by reference herein. A non-limiting list of preferred therapeutic oligonucleotides includes antisense bcl-2 oligonucleotides, antisense HIF-1α oligonucleotides, antisense survivin oligonucleotides, antisense ErbB3 oligonucleotides, antisense PIK3CA oligonucleotides, antisense HSP27 oligonucleotides, antisense androgen receptor oligonucleotides, antisense Gli2 oligonucleotides, and anti sense beta-catenin oligonucleotides.

More preferably, the oligonucleotides according to the invention described herein include phosphorothioate backbone and LNA.

In one preferred embodiment, the oligonucleotide can be, for example, antisense survivin LNA, antisense ErbB3 LNA, or antisense HIF1-α LNA.

In another preferred embodiment, the oligonucleotide can be, for example, an oligonucleotide that has the same or substantially similar nucleotide sequence as does Genasense® (a/k/a oblimersen sodium, produced by Genta Inc., Berkeley Heights, N.J.). Genasense® is an 18-mer phosphorothioate antisense oligonucleotide (SEQ ID NO: 4), that is complementary to the first six codons of the initiating sequence of the human bcl-2 mRNA (human bcl-2 mRNA is art-known, and is described, e.g., as SEQ ID NO: 19 in U.S. Pat. No. 6,414,134, incorporated by reference herein).

Preferred embodiments contemplated include:

(i) Antisense Survivin LNA Oligomer (SEQ ID NO: 1)
^{m}Cₛ-Tₛ-^{m}Cₛ-Aₛ-aₛ-tₛ-cₛ-cₛ-aₛ-tₛ-gₛ-gₛ-^{m}Cₛ-Aₛ-Gₛ-c;
where the upper case letter represents LNA, the “s” represents a phosphorothioate backbone;

(ii) Antisense Bcl2 siRNA:

| | |
|---|---|
| SENSE | |
| 5′-gcaugcggccucuguuugadTdT-3′ | (SEQ ID NO: 2) |
| ANTISENSE | |
| 3′-dTdTcguacgccggagacaaacu-5′ | (SEQ ID NO: 3) |

where dT represents DNA;

(iii) Genasense (Phosphorothioate Antisense Oligonucleotide): (SEQ ID NO: 4)
tₛ-cₛ-tₛ-cₛ-cₛ-cₛ-aₛ-gₛ-cₛ-gₛ-tₛ-gₛ-cₛ-gₛ-cₛ-cₛ-cₛ-aₛ-t
where the lower case letter represents DNA and “s” represents phosphorothioate backbone;

(iv) Antisense HIF1α LNA Oligomer (SEQ ID NO: 5)
TₛGₛGₛcₛaₛaₛgₛcₛaₛtₛcₛcₛTₛGₛTₛa
where the upper case letter represents LNA and the “s” represents phosphorothioate backbone.

(v) Antisense ErbB3 LNA Oligomer (SEQ ID NO: 6)
TₛAₛGₛcₛcₛtₛgₛtₛcₛaₛcₛtₛtₛ^{Me}CₛTₛ^{Me}Cₛ
where the upper case letter represents LNA and the “s” represents phosphorothioate backbone.

(vi) Antisense ErbB3 LNA Oligomer (SEQ ID NO: 7)
Gₛ^{Me}CₛTₛcₛcₛaₛgₛaₛcₛaₛtₛcₛaₛ^{Me}CₛTₛ^{Me}C
where the upper case letter represents LNA and the “s” represents phosphorothioate backbone.

(vii) Antisense PIK3CA LNA Oligomer (SEQ ID NO: 8)
AₛGₛ^{Me}CₛcₛaₛtₛtₛcₛaₛtₛtₛcₛcₛAₛ^{Me}Cₛ^{Me}C
where the upper case letter represents LNA and the “s” represents phosphorothioate backbone.

(viii) Antisense PIK3CA LNA Oligomer (SEQ ID NO: 9)
TₛTₛAₛtₛtₛgₛtₛgₛcₛaₛtₛcₛtₛ^{Me}CₛAₛG
where the upper case letter represents LNA and the “s” represents phosphorothioate backbone.

(ix) Antisense HSP27 LNA Oligomer (SEQ ID NO: 10)
C_{S}G_{S}T_{S}g_{S}t_{S}a_{S}t_{S}t_{S}t_{S}c_{S}c_{S}g_{S}c_{S}G_{S}T_{S}G
where the upper case letter represents LNA and the “s” represents phosphorothioate backbone.

(x) Antisense HSP27 LNA Oligomer (SEQ ID NO: 11)
GₛGₛ^{Me}CₛaₛcₛaₛgₛcₛcₛaₛgₛtₛgₛGₛ^{Me}CₛG
where the upper case letter represents LNA and the “s” represents phosphorothioate backbone.

(xi) Antisense Androgen Receptor LNA Oligomer (SEQ ID NO: 12)
^{Me}Cₛ^{Me}Cₛ^{Me}CₛaₛaₛgₛgₛcₛaₛcₛtₛgₛcₛAₛGₛA
where the upper case letter represents LNA and the “s” represents phosphorothioate backbone.

(xii) Antisense Androgen Receptor LNA Oligomer (SEQ ID NO: 13)
A_{S}^{Me}C_{S}^{Me}CₛaₛaₛgₛtₛtₛtₛcₛtₛtₛcₛAₛGₛ^{Me}C
where the upper case letter represents LNA and the “s” represents phosphorothioate backbone.

(xiii) Antisense GLI2 LNA Oligomer (SEQ ID NO: 14)
^{Me}C_{S}T_{S}^{Me}C_{S}c_{S}t_{S}t_{S}g_{S}g_{S}t_{S}g_{S}c_{S}a_{S}g_{S}T_{S}^{Me}C_{S}T
where the upper case letter represents LNA and the “s” represents phosphorothioate backbone.

(xiv) Antisense GLI2 LNA oligomer (SEQ ID NO: 15)
Tₛ^{Me}CₛAₛgₛaₛtₛtₛcₛaₛaₛaₛcₛ^{Me}Cₛ^{Me}CₛA
where the upper case letter represents LNA and the “s” represents phosphorothioate backbone

(xv) Antisense Beta-Catenin LNA Oligomer (SEQ ID NO: 16)
GₛTₛGₛtₛtₛcₛtₛaₛcₛaₛcₛcₛaₛTₛTₛA
where the upper case letter represents LNA and the “s” represents phosphorothioate backbone.

Lower case letters represent DNA units, bold upper case letters represent LNA such as 13-D-oxy-LNA units. All cytosine bases in the LNA monomers are 5-methylcytosine. Subscript “s” represents phosphorothioate linkage.

LNA includes 2′-O, 4′-C methylene bicyclonucleotide as shown below:

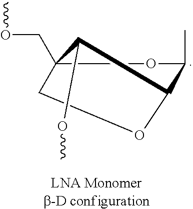

See detailed description of Survivin LNA disclosed in U.S. patent application Ser. Nos. 11/272,124, entitled “LNA Oligonucleotides and the Treatment of Cancer” and 10/776,934, entitled “Oligomeric Compounds for the Modulation Survivin Expression”, the contents of each of which is incorporated herein by reference. See also U.S. Pat. No. 7,589,190 and U.S. Patent Publication No. 2004/0096848 for HIF-1α modulation; U.S. Patent Publication No. 2008/0318894 and PCT/US09/063,357 for ErbB3 modulation; U.S. Patent Publication No. 2009/0192110 for PIK3CA modulation; PCT/IB09/052,860 for HSP27 modulation; U.S. Patent Publication No. 2009/0181916 for Androgen Receptor modulation; and U.S. Provisional Application No. 61/081,135 and PCT Application No. PCT/IB09/006,407, entitled “RNA Antagonists Targeting GLI2”; and U.S. Patent Publication Nos. 2009/0005335 and 2009/0203137 for Beta Catenin modulation; the contents of each which are also incorporated herein by reference. Additional examples of suitable target genes are described in WO 03/74654, PCT/US03/05028, and U.S. patent application Ser. No. 10/923,536, the contents of which are incorporated by reference herein.

In a further embodiment, the nanoparticle described herein can include oligonucleotides releasably linked to an endosomal release-promoting group. The endosomal release-promoting groups such as histidine-rich peptides can destabilize/disrupt the endosomal membrane, thereby facilitating cytoplasmic delivery of therapeutic agents. Histidine-rich peptides enhance endosomal release of oligonucleotides to the cytoplasm. Then, the intracellularly released oligonucleotides can translocate to the nucleus. Additional details of oligonucleotide-histidine rich peptide conjugates are described in U.S. Provisional Patent Application Nos. 61/115,350 and 61/115,326, filed Nov. 17, 2008, and PCT Patent Application No. ______, filed on even date, and entitled “Releasable Conjugates For Nucleic Acids Delivery Systems”, the contents of each of which are incorporated herein by reference.

### 6. Targeting Groups

Optionally/preferably, the nanoparticle compositions described herein further include a targeting ligand for a specific cell of tissue type. The targeting group can be attached to any component of a nanoparticle composition (e.g., fusogenic lipids, PEG-lipids, etc, preferably releasable polymeric lipids of Formula (I)) using a linker molecule, such as an amide, amido, carbonyl, ester, peptide, disulphide, silane, nucleoside, abasic nucleoside, polyether, polyamine, polyamide, peptide, carbohydrate, lipid, polyhydrocarbon, phosphate ester, phosphoramidate, thiophosphate, alkylphosphate, maleimidyl linker or photolabile linker. Any known techniques in the art can be used for conjugating a targeting group to any component of nanoparticle composition without undue experimentation.

For example, targeting agents can be attached to the polymeric portion of PEG lipids, including compounds of Formula (I), to guide the nanoparticles to the target area in vivo. The targeted delivery of the nanoparticle described herein enhances cellular uptake of the nanoparticles encapsulating therapeutic nucleic acids, thereby improving therapeutic efficacies of the nanoparticles. In certain aspects, some cell penetrating peptides can be replaced with a variety of targeting peptides for targeted delivery to the tumor site.

In one preferred aspect of the invention, the targeting moiety, such as a single chain antibody (SCA) or single-chain antigen-binding antibody, monoclonal antibody, cell adhesion peptides such as RGD peptides and Selectin, cell penetrating peptides (CPPs) such as TAT, Penetratin and (Arg)₉, receptor ligands, targeting carbohydrate molecules or lectins allows nanoparticles to be specifically directed to targeted regions. See J Pharm Sci. 2006 September; 95(9):1856-72 Cell adhesion molecules for targeted drug delivery, the contents of which are incorporated herein by reference.

Preferred targeting moieties include single-chain antibodies (SCAs) or single-chain variable fragments of antibodies (sFv). The SCA contains domains of antibodies which can bind or recognize specific molecules of targeting tumor cells. In addition to maintaining an antigen binding site, a SCA conjugated to a PEG-lipid can reduce antigenicity and increase the half life of the SCA in the bloodstream.

The terms “single chain antibody” (SCA), “single-chain antigen-binding molecule or antibody” or “single-chain Fv” (sFv) are used interchangeably. The single chain antibody has binding affinity for the antigen. Single chain antibody (SCA) or single-chain Fvs can and have been constructed in several ways. A description of the theory and production of single-chain antigen-binding proteins is found in commonly assigned U.S. patent application Ser. No. 10/915,069 and U.S. Pat. No. 6,824,782, the contents of each of which are incorporated by reference herein.

Typically, SCA or Fv domains can be selected among monoclonal antibodies known by their abbreviations in the literature as 26-10, MOPC 315, 741F8, 520C9, McPC 603, D1.3, murine phOx, human phOx, RFL3.8 sTCR, 1A6, Se155-4, 18-2-3,4-4-20,7A4-1, B6.2, CC49,3C2,2c, MA-15C5/K₁₂G_{O}, Ox, etc. (see, Huston, J. S. et al., Proc. Natl. Acad. Sci. USA 85:5879-5883 (1988); Huston, J. S. et al., SIM News 38(4) (Supp):11 (1988); McCartney, J. et al., ICSU Short Reports 10:114 (1990); McCartney, J. E. et al., unpublished results (1990); Nedelman, M. A. et al., J. Nuclear Med. 32 (Supp.):1005 (1991); Huston, J. S. et al., In: Molecular Design and Modeling: Concepts and Applications, Part B, edited by J. J. Langone, Methods in Enzymology 203:46-88 (1991); Huston, J. S. et al., In: Advances in the Applications of Monoclonal Antibodies in Clinical Oncology, Epenetos, A. A. (Ed.), London, Chapman & Hall (1993); Bird, R. E. et al., Science 242:423-426 (1988); Bedzyk, W. D. et al., J. Biol. Chem., 265:18615-18620 (1990); Colcher, D. et al., J. Nat. Cancer Inst. 82:1191-1197 (1990); Gibbs, R. A. et al., Proc. Natl. Acad. Sci. USA 88:4001-4004 (1991); Milenic, D. E. et al., Cancer Research 51:6363-6371 (1991); Pantoliano, M. W. et al., Biochemistry 30:10117-10125 (1991); Chaudhary, V. K. et al., Nature 339:394-397 (1989); Chaudhary, V. K. et al., Proc. Natl. Acad. Sci. USA 87:1066-1070 (1990); Batra, J. K. et al., Biochem. Biophys. Res. Comm. 171:1-6 (1990); Batra, J. K. et al., J. Biol. Chem. 265:15198-15202 (1990); Chaudhary, V. K. et al., Proc. Natl. Acad. Sci. USA 87:9491-9494 (1990); Batra, J. K. et al., Mol. Cell. Biol. 11:2200-2205 (1991); Brinkmann, U. et al., Proc. Natl. Acad. Sci. USA 88:8616-8620 (1991); Seetharam, S. et al., J. Biol. Chem. 266:17376-17381 (1991); Brinkmann, U. et al., Proc. Natl. Acad. Sci. USA 89:3075-3079 (1992); Glockshuber, R. et al., Biochemistry 29:1362-1367 (1990); Skerra, A. et al., Bio/Technol. 9:273-278 (1991); Pack, P. et al., Biochemistry 31:1579-1534 (1992); Clackson, T. et al., Nature 352:624-628 (1991); Marks, J. D. et al., J. Mol. Biol. 222:581-597 (1991); Iverson, B. L. et al., Science 249:659-662 (1990); Roberts, V. A. et al., Proc. Natl. Acad. Sci. USA 87:6654-6658 (1990); Condra, J. H. et al., J. Biol. Chem. 265:2292-2295 (1990); Laroche, Y. et al., J. Biol. Chem. 266:16343-16349 (1991); Holvoet, P. et al., J. Biol. Chem. 266:19717-19724 (1991); Anand, N. N. et al., J. Biol. Chem. 266:21874-21879 (1991); Fuchs, P. et al., Biol Technol. 9:1369-1372 (1991); Breitling, F. et al., Gene 104:104-153 (1991); Seehaus, T. et al., Gene 114:235-237 (1992); Takkinen, K. et al., Protein Engng. 4:837-841 (1991); Dreher, M. L. et al., J. Immunol. Methods 139:197-205 (1991); Mottez, E. et al., Eur. J. Immunol. 21:467-471 (1991); Traunecker, A. et al., Proc. Natl. Acad. Sci. USA 88:8646-8650 (1991); Traunecker, A. et al., EMBO J. 10:3655-3659 (1991); Hoo, W. F. S. et al., Proc. Natl. Acad. Sci. USA 89:4759-4763 (1993)). Each of the foregoing publications is incorporated herein by reference.

A non-limiting list of targeting groups includes vascular endothelial cell growth factor, FGF2, somatostatin and somatostatin analogs, transferrin, melanotropin, ApoE and ApoE peptides, von Willebrand's Factor and von Willebrand's Factor peptides, adenoviral fiber protein and adenoviral fiber protein peptides, PD1 and PD1 peptides, EGF and EGF peptides, ROD peptides, folate, anisamide, etc. Other optional targeting agents appreciated by artisans in the art can be also employed in the nanoparticles described herein.

In one preferred embodiment, the targeting agents useful for the compounds described herein include single chain antibody (SCA), RGD peptides, selectin, TAT, penetratin, (Arg)₉, folic acid, anisamide, etc., and some of the preferred structures of these agents are:

| | | |
|---|---|---|
| C-TAT: | CYGRKKRRQRRR; | (SEQ ID NO: 17) |
| C-(Arg)₉: | CRRRRRRRRR; | (SEQ ID NO: 18) |

RGD can be linear or cyclic:

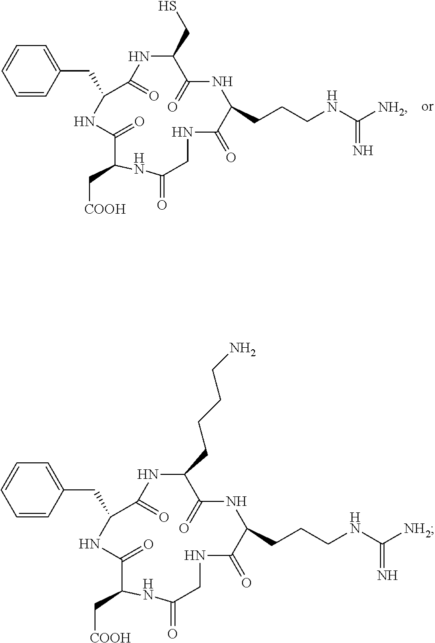

Folic acid is a residue of

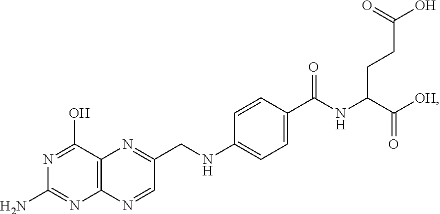

and

Anisamide is p-MeO-Ph-C(═O)OH.

Arg₉ can include a cysteine for conjugating such as CRRRRRRRRR and TAT can add an additional cysteine at the end of the peptide such as CYGRKKRRQRRRC,

For purpose of the current invention, the abbreviations used in the specification and figures represent the following structures:

(i) C-diTAT (SEQ ID NO: 19)=CYGRKKRRQRRRYGRKKRRQRRR—NH₂;

(ii) Linear RGD (SEQ ID NO: 20)=RGDC;

(iii) Cyclic RGD (SEQ ID NO: 21 and SEQ ID NO: 22)=c-RGDFC or c-RGDFK;

(iv) RGD-TAT (SEQ ID NO: 23)=CYGRKKRRQRRRGGGRGDS-NH₂; and

(v) Arg₉ (SEQ ID NO: 24)=RRRRRRRRR.

Alternatively, the targeting group include sugars and carbohydrates such as galactose, galactosamine, and N-acetyl galactosamine; hormones such as estrogen, testosterone, progesterone, glucocortisone, adrenaline, insulin, glucagon, cortisol, vitamin D, thyroid hormone, retinoic acid, and growth hormones; growth factors such as VEGF, EGF, NGF, and PDGF; neurotransmitters such as GABA, Glutamate, acetylcholine; NOW; inostitol triphosphate; epinephrine; norepinephrine; Nitric Oxide, peptides, vitamins such as folate and pyridoxine, drugs, antibodies and any other molecule that can interact with a cell surface receptor in vivo or in vitro.

### D. Preparation of Nanoparticles

The nanoparticle described herein can be prepared by any art-known process without undue experimentation.

For example, the nanoparticle can be prepared by providing nucleic acids such as oligonucleotides in an aqueous solution (or an aqueous solution without nucleic acids for comparison study) in a first reservoir, and providing an organic lipid solution containing the nanoparticle composition described herein in a second reservoir, and mixing the aqueous solution with the organic lipid solution such that the organic lipid solution mixes with the aqueous solution to produce nanoparticles encapsulating the nucleic acids. Details of the process are described in U.S. Patent Publication No. 2004/0142025, the contents of which are incorporated herein by reference.

Alternatively, the nanoparticles described herein can be prepared using any methods known in the art including, e.g., a detergent dialysis method or a modified reverse-phase method which utilizes organic solvents to provide a single phase during mixing the components. In a detergent dialysis method, nucleic acids (i.e., siRNA) are contacted with a detergent solution of cationic lipids to form a coated nucleic acid complex.

In one embodiment of the invention, the cationic lipids and nucleic acids such as oligonucleotides are combined to produce a charge ratio of from about 1:20 to about 20:1, preferably in a ratio of from about 1:5 to about 5:1, and more preferably in a ratio of from about 1:2 to about 2:1.

In one preferred embodiment, the nanoparticle described herein can be carried out using a dual pump system. Generally, the process includes providing an aqueous solution containing nucleic acids in a first reservoir and a lipid solution containing the nanoparticle composition described in a second reservoir. The two solutions are mixed using a dual pump system to provide nanoparticles. The resulting mixed solution is subsequently diluted with an aqueous buffer and the nanoparticles formed can be purified and/or isolated by dialysis. The nanoparticles can be further processed to be sterilized by filtering through a 0.22 μm filter.

The nanoparticles containing nucleic acids range from about 5 to about 300 nm in diameter. Preferably, the nanoparticles have a median diameter of less than about 150 nm, more preferably a diameter of less than about 100 nm. A majority of the nanoparticles have a median diameter of about 30 to 100 nm (e.g., 59.5, 66, 68, 76, 80, 93, 96 nm), preferably 60-95 nm. The nanoparticles of the present invention are desirably uniform in size as shown by polydispersity.

Optionally, the nanoparticles can be sized by any methods known in the art. The sizing may be conducted in order to achieve a desired size range and relatively narrow distribution of nanoparticle sizes. Several techniques are available for sizing the nanoparticles to a desired size. See, for example, U.S. Pat. No. 4,737,323, the contents of which are incorporated herein by reference.

The present invention provides methods for preparing serum-stable nanoparticles such that the nucleic acid (e.g., LNA or siRNA) is encapsulated in a lipid bilayer and is protected from degradation. The nucleic acids when present in the nanoparticles of the present invention are resistant to aqueous solution degradation with a nuclease.

Additionally, the nanoparticles prepared according to the present invention are preferably neutral or positively-charged at physiological pH.

The nanoparticle or nanoparticle complex prepared using the nanoparticle composition described herein includes: (i) a cationic lipid; (ii) a neutral lipid (fusogenic lipid); (iii) a releasable polymeric lipid of Formula (I), and (iv) nucleic acids such as an oligonucleotide.

In one embodiment, the nanoparticle composition includes a mixture of

a mixture of a cationic lipid, a diacylphosphatidylethanolamine, a compound of Formula (I), and cholesterol;

a mixture of a cationic lipid, a diacylphosphatidylcholine, a compound of Formula (I), and cholesterol;

a mixture of a cationic lipid, a diacylphosphatidylethanolamine, a diacylphosphatidylcholine, a compound of Formula (I), and cholesterol; and

a mixture of a cationic lipid, a diacylphosphatidylethanolamine, a compound of Formula (I), a PEG conjugated to ceramide (PEG-Cer), and cholesterol.

Additional nanoparticle compositions can be prepared by modifying compositions containing art-known cationic lipid(s). Nanoparticle compositions containing a compound of Formula (I) can be modified by adding art-known cationic lipids. See art-known compositions described in Table IV of US Patent Application Publication No. 2008/0020058, the contents of which are incorporated herein by reference.

A non-limiting list of nanoparticle compositions is contemplated to prepare nanoparticles as set forth in Table 3.

TABLE 3

| Sample# | Nanoparticle Composition | Molar Ratio | Oligo |
|---|---|---|---|
| 1 | Cationic Lipid 1:DOPE:DSPC:Chol:Compd 10 | 15:15:20:40:10 | Oligo-1 |
| 2 | Cationic Lipid 1:DOPE:DSPC:Chol:Compd 10 | 15:5:20:50:10 | Oligo-1 |
| 3 | Cationic Lipid 1:DOPE:DSPC:Chol:Compd 10 | 25:15:20:30:10 | Oligo-1 |
| 4 | Cationic Lipid 1:EPC:Chol:Compd 10 | 20:47:30:3 | Oligo-1 |
| 5 | Cationic Lipid 1:DOPE:Chol:Compd 10 | 17:60:20:3 | Oligo-1 |
| 6 | Cationic Lipid 1:DOPE:Compd 10 | 20:78:2 | Oligo-1 |
| 7 | Cationic Lipid 1:DOPE:Chol:Compd 10 | 17:60:20:3 | Oligo-2 |
| 8 | Cationic Lipid 1:DOPE:Chol:Compd 10 | 18:60:20:2 | Oligo-2 |
| 9 | Cationic Lipid 1:DOPE:Chol:Compd 10 | 18:52:20:10 | Oligo-2 |
| 10 | Cationic Lipid 1:DOPE:Chol:Compd 10 | 18:57:20:5 | Oligo-2 |

In one embodiment, a cationic lipid 1: DOPE: cholesterol: compound 10 in the nanoparticle is present in a molar ratio of about 18%:52%:20%:10%, respectively. (Sample No. 9)

In another embodiment, the nanoparticle contains a cationic lipid (compound 1), DOPE, cholesterol and compound 10 in a molar ratio of about 18%:57%:20%:5% of the total lipid present in the nanoparticle composition. (Sample No. 10)

These nanoparticle compositions preferably contain a releasable polymeric lipid having the structure:

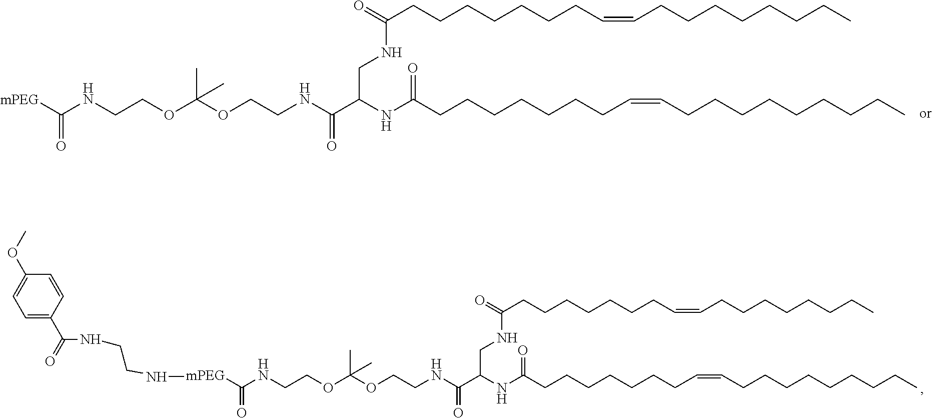

wherein the polymer portion of the PEG lipid has a number average weight of about 2,000 daltons.

In one embodiment, the cationic lipid contained in the compositions has the structure:

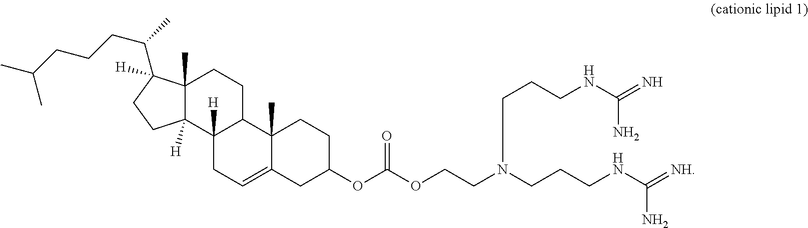

The molar ratio as used herein refers to the amount relative to the total lipid present in the nanoparticle composition.

### E. Methods of Treatment

The nanoparticles described herein can be employed in the treatment for preventing, inhibiting, reducing or treating any trait, disease or condition that is related to or responds to the levels of target gene expression in a cell or tissue, alone or in combination with other therapies. The methods include administering the nanoparticles described herein to a mammal in need thereof.

One aspect of the present invention provides methods of introducing or delivering therapeutic agents such as nucleic acids/oligonucleotides into a mammalian cell in vivo and/or in vitro.

The method according to the present invention includes contacting a cell with the compounds described herein. The delivery can be made in vivo as part of a suitable pharmaceutical composition or directly to the cells in an ex vivo or in vitro environment.

The present invention is useful for introducing oligonucleotides to a mammal. The compounds described herein can be administered to a mammal, preferably human.

According to the present invention, the present invention preferably provides methods of inhibiting, or downregulating (or modulating) gene expression in mammalian cells or tissues. The downregulation or inhibition of gene expression can be achieved in vivo, ex vivo and/or in vitro. The methods include contacting human cells or tissues with nanoparticles encapsulating nucleic acids or administering the nanoparticles to a mammal in need thereof. Once the contacting has occurred, successful inhibition or down-regulation of gene expression such as in mRNA or protein levels shall be deemed to occur when at least about 10%, preferably at least about 20% or higher (e.g., at least about 25%, 30%, 40%, 50%, 60%) is realized in vivo, ex vivo or in vitro when compared to that observed in the absence of the nanoparticles described herein.

For purposes of the present invention, “inhibiting” or “downregulating” shall be understood to mean that the expression of a target gene, or level of RNAs or equivalent RNAs encoding one or more protein subunits, or activity of one or more protein subunits is reduced when compared to that observed in the absence of the nanoparticles described herein.

In one preferred embodiment, a target gene includes, for example, but is not limited to, oncogenes, pro-angiogenesis pathway genes, pro-cell proliferation pathway genes, viral infectious agent genes, and pro-inflammatory pathway genes.

Preferably, gene expression of a target gene is inhibited in cancer cells or tissues, for example, brain, breast, colorectal, gastric, lung, mouth, pancreatic, prostate, skin or cervical cancer cells. The cancer cells or tissues can be from one or more of the following: solid tumors, lymphomas, small cell lung cancer, acute lymphocytic leukemia (ALL), pancreatic cancer, glioblastoma, ovarian cancer, gastric cancer, breast cancer, colorectal cancer, prostate cancer, cervical cancer, brain tumors, KB cancer, lung cancer, colon cancer, epidermal cancer, etc.

In one particular embodiment, the nanoparticles according to the methods described herein include, for example, antisense bcl-2 oligonucleotides, antisense HIF-1α oligonucleotides, antisense survivin oligonucleotides, antisense ErbB3 oligonucleotides, antisense PIK3CA oligonucleotides, antisense HSP27 oligonucleotides, antisense androgen receptor oligonucleotides, antisense Gli2 oligonucleotides, and antisense beta-catenin oligonucleotides.

According to the present invention, the nanoparticles can include oligonucleotides (SEQ ID NO: 1, SEQ ID NOs 2 and 3, SEQ ID NO:3, SEQ ID NO: 4, SEQ ID NO: 5, SEQ ID NO: 6, SEQ ID NO: 7, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16 in which each nucleic acid is a naturally occurring or modified nucleic acid) can be used. The therapy contemplated herein uses nucleic acids encapsulated in the aforementioned nanoparticle. In one embodiment, therapeutic nucleotides containing eight or more consecutive antisense nucleotides can be employed in the treatment.

Alternatively, there are also provided methods of treating a mammal. The methods include administering an effective amount of a pharmaceutical composition containing a nanoparticle described herein to a patient in need thereof. The efficacy of the methods would depend upon efficacy of the nucleic acids for the condition being treated. The present invention provides methods of treatment for various medical conditions in mammals. The methods include administering, to the mammal in need of such treatment, an effective amount of a nanoparticle containing encapsulated therapeutic nucleic acids. The nanoparticles described herein are useful for, among other things, treating diseases such as (but not limited to) cancer, inflammatory disease, and autoimmune disease.

In one embodiment, there are also provided methods of treating a patient having a malignancy or cancer, comprising administering an effective amount of a pharmaceutical composition containing the nanoparticle described herein to a patient in need thereof. The cancer being treated can be one or more of the following: solid tumors, lymphomas, small cell lung cancer, acute lymphocytic leukemia (ALL), pancreatic cancer, glioblastoma, ovarian cancer, gastric cancers, colorectal cancer, prostate cancer, cervical cancer, brain tumors, KB cancer, lung cancer, colon cancer, epidermal cancer, etc. The nanoparticles are useful for treating neoplastic disease, reducing tumor burden, preventing metastasis of neoplasms and preventing recurrences of tumor/neoplastic growths in mammals by downregulating gene expression of a target gene. For example, the nanoparticles are useful in the treatment of metastatic disease (i.e. cancer with metastasis into the liver).

In yet another aspect, the present invention provides methods of inhibiting the growth or proliferation of cancer cells in vivo or in vitro. The methods include contacting cancer cells with the nanoparticle described herein. In one embodiment, the present invention provides methods of inhibiting the growth of cancer in vivo or in vitro wherein the cells express ErbB3 gene.

In another aspect, the present invention provides a means to deliver nucleic acids (e.g., antisense ErbB3 LNA oligonucleotides) inside a cancer cell where it can bind to ErbB3 mRNA, e.g., in the nucleus. As a consequence, the ErbB3 protein expression is inhibited, which inhibits the growth of the cancer cells. The methods introduce oligonucleotides (e.g. antisense oligonucleotides including LNA) to cancer cells and reduce target gene (e.g., survivin, HIF-1α or ErbB3) expression in the cancer cells or tissues.

Alternatively, the present invention provides methods of modulating apoptosis in cancer cells. In yet another aspect, there are also provided methods of increasing the sensitivity of cancer cells or tissues to chemotherapeutic agents in vivo or in vitro.

In yet another aspect, there are provided methods of killing tumor cells in vivo or in vitro. The methods include introducing the compounds described herein to tumor cells to reduce gene expression such as ErbB3 gene and contacting the tumor cells with an amount of at least one anticancer agent (e.g., a chemotherapeutic agent) sufficient to kill a portion of the tumor cells. Thus, the portion of tumor cells killed can be greater than the portion which would have been killed by the same amount of the chemotherapeutic agent in the absence of the nanoparticles described herein.

In a further aspect of the invention, an anticancer/chemotherapeutic agent can be used in combination, simultaneously or sequentially, with the compounds described herein. The compounds described herein can be administered prior to, or concurrently with, the anticancer agent, or after the administration of the anticancer agent. Thus, the nanoparticles described herein can be administered prior to, during, or after treatment of the chemotherapeutic agent.

Still further aspects include combining the compound of the present invention described herein with other anticancer therapies for synergistic or additive benefit.

Alternatively, the nanoparticle composition described herein can be used to deliver a pharmaceutically active agent, preferably having a negative charge or a neutral charge to a mammal. The nanoparticle encapsulating pharmaceutically active agents/compounds can be administered to a mammal in need thereof. The pharmaceutically active agents/compounds include small molecular weight molecules. Typically, the pharmaceutically active agents have a molecular weight of less than about 1,500 daltons (i.e., less than 1,000 daltons).

In a further embodiment, the compounds described herein can be used to deliver nucleic acids, a pharmaceutically active agent, or in combination thereof.

In yet a further embodiment, the nanoparticle associated with the treatment can contain a mixture of one or more therapeutic nucleic acids (either the same or different, for example, the same or different oligonucleotides), and/or one or more pharmaceutically active agents for synergistic application.

### F. Pharmaceutical Compositions/Formulations of Nanoparticles

Pharmaceutical compositions/formulations including the nanoparticles described herein may be formulated in conjunction with one or more physiologically acceptable carriers comprising excipients and auxiliaries which facilitate processing of the active compounds into preparations which can be used pharmaceutically. Proper formulation is dependent upon the route of administration chosen, i.e., whether local or systemic treatment is treated.

Suitable forms, in part, depend upon the use or the route of entry, for example oral, transdermal, or injection. Factors for considerations known in the art for preparing proper formulations include, but are not limited to, toxicity and any disadvantages that would prevent the composition or formulation from exerting its effect.

Administration of pharmaceutical compositions of nanoparticles described herein may be oral, pulmonary, topical or parentarel. Topical administration includes, without limitation, administration via the epidermal, transdermal, ophthalmic routes, including via mucous membranes, e.g., including vaginal and rectal delivery. Parenteral administration, including intravenous, intraarterial, subcutaneous, intraperitoneal or intramuscular injection or infusion, is also contemplated.

In one preferred embodiment, the nanoparticles containing therapeutic oligonucleotides are administered intravenously (i.v.) or intraperitoneally (i.p.). Parenteral routes are preferred in many aspects of the invention.

For injection, including, without limitation, intravenous, intramuscular and subcutaneous injection, the nanoparticles of the invention may be formulated in aqueous solutions, preferably in physiologically compatible buffers such as physiological saline buffer or polar solvents including, without limitation, a pyrrolidone or dimethylsulfoxide.

The nanoparticles may also be formulated for bolus injection or for continuous infusion. Formulations for injection may be presented in unit dosage form, e.g., in ampoules or in multi-dose containers. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water soluble form. Aqueous injection suspensions may contain substances that modulate the viscosity of the suspension, such as sodium carboxymethyl cellulose, sorbitol, or dextran. Optionally, the suspension may also contain suitable stabilizers and/or agents that increase the concentration of the nanoparticles in the solution. Alternatively, the nanoparticles may be in powder form for constitution with a suitable vehicle, e.g., sterile, pyrogen-free water, before use.

For oral administration, the nanoparticles described herein can be formulated by combining the nanoparticles with pharmaceutically acceptable carriers well-known in the art. Such carriers enable the nanoparticles of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars (for example, lactose, sucrose, mannitol, or sorbitol), cellulose preparations such as maize starch, wheat starch, rice starch and potato starch and other materials such as gelatin, gum tragacanth, methyl cellulose, hydroxypropylmethylcellulose, sodium carboxymethylcellulose, and/or polyvinylpyrrolidone (PVP). If desired, disintegrating agents may be added, such as cross-linked polyvinyl pyrrolidone, agar, or alginic acid. A salt such as sodium alginate may also be used.

For administration by inhalation, the nanoparticles of the present invention can conveniently be delivered in the form of an aerosol spray using a pressurized pack or a nebulizer and a suitable propellant.

The nanoparticles may also be formulated in rectal compositions such as suppositories or retention enemas, using, e.g., conventional suppository bases such as cocoa butter or other glycerides.

In addition to the formulations described previously, the nanoparticles may also be formulated as depot preparations. Such long acting formulations may be administered by implantation (for example, subcutaneously or intramuscularly) or by intramuscular injection. A nanoparticle of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Additionally, the nanoparticles may be delivered using a sustained-release system, such as semi-permeable matrices of solid hydrophobic polymers containing the nanoparticles. Various sustained-release materials have been established and are well known by those skilled in the art.

In addition, antioxidants and suspending agents can be used in the pharmaceutical compositions of the nanoparticles described herein.

### G. Dosages

Determination of doses adequate to inhibit the expression of one or more preselected genes, such as a therapeutically effective amount in the clinical context, is well within the capability of those skilled in the art, especially in light of the disclosure herein.

For any therapeutic nucleic acids used in the methods of the invention, the therapeutically effective amount can be estimated initially from in vitro assays. Then, the dosage can be formulated for use in animal models so as to achieve a circulating concentration range that includes the effective dosage. Such information can then be used to more accurately determine dosages useful in patients.

The amount of the pharmaceutical composition that is administered will depend upon the potency of the nucleic acids included therein. Generally, the amount of the nanoparticles containing nucleic acids used in the treatment is that amount which effectively achieves the desired therapeutic result in mammals. Naturally, the dosages of the various nanoparticles will vary somewhat depending upon the nucleic acids (or pharmaceutically active agents) encapsulated therein (e.g., oligonucleotides). In addition, the dosage, of course, can vary depending upon the dosage form and route of administration. In general, however, the nucleic acids encapsulated in the nanoparticles described herein can be administered in amounts ranging from about 0.1 to about 1 g/kg/week, preferably from about 1 to about 500 mg/kg and more preferably from 1 to about 100 mg/kg (i.e., from about 3 to about 90 mg/kg/dose).

The range set forth above is illustrative and those skilled in the art will determine the optimal dosing based on clinical experience and the treatment indication. Moreover, the exact formulation, route of administration and dosage can be selected by the individual physician in view of the patient's condition. Additionally, toxicity and therapeutic efficacy of the nanoparticles described herein can be determined by standard pharmaceutical procedures in cell cultures or experimental animals using methods well-known in the art.

Alternatively, an amount of from about 1 mg to about 100 mg/kg/dose (0.1 to 100 mg/kg/dose) can be used in the treatment depending on potency of the nucleic acids. Dosage unit forms generally range from about 1 mg to about 60 mg of an active agent, oligonucleotides.

In one embodiment, the treatment of the present invention includes administering the nanoparticles described herein in an amount of from about 1 to about 60 mg/kg/dose (from about 25 to 60 mg/kg/dose, from about 3 to about 20 mg/kg/dose), such as 60, 45, 35, 30, 25, 15, 5 or 3 mg/kg/dose (either in a single or multiple dose regime) to a mammal. For example, the nanoparticles described herein can be administered introvenously in an amount of 5, 25, 30, or 60 mg/kg/dose at q3d×9. For another example, the treatment protocol includes administering an antisense oligonucleotide in an amount of from about 4 to about 18 mg/kg/dose weekly, or about 4 to about 9.5 mg/kg/dose weekly (e.g., about 8 mg/kg/dose weekly for 3 weeks in a six week cycle).

Alternatively, the delivery of the oligonucleotide encapsulated within the nanoparticles described herein includes contacting a concentration of oligonucleotides of from about 0.1 to about 1000 μM, preferably from about 10 to about 1500 μM (i.e. from about 10 to about 1000 μM, from about 30 to about 1000 μM) with tumor cells or tissues in vivo, ex vivo or in vitro.

The compositions may be administered once daily or divided into multiple doses which can be given as part of a multi-week treatment protocol. The precise dose will depend on the stage and severity of the condition, the susceptibility of the disease such as tumor to the nucleic acids, and the individual characteristics of the patient being treated, as will be appreciated by one of ordinary skill in the art.

In all aspects of the invention where nanoparticles are administered, the dosage amount mentioned is based on the amount of oligonucleotide molecules rather than the amount of nanoparticles administered.

It is contemplated that the treatment will be given for one or more days until the desired clinical result is obtained. The exact amount, frequency and period of administration of the nanoparticles encapsulating therapeutic nucleic acids (or pharmaceutically active agents) will vary, of course, depending upon the sex, age and medical condition of the patent as well as the severity of the disease as determined by the attending clinician.

Still further aspects include combining the nanoparticles of the present invention described herein with other anticancer therapies for synergistic or additive benefit.

### EXAMPLES

The following examples serve to provide further appreciation of the invention but are not meant in any way to restrict the effective scope of the invention. In the examples, all synthesis reactions are run under an atmosphere of dry nitrogen or argon. N-(3-aminopropyl)-1,3-propanediamine), BOC—ON, LiOCl₄, Cholesterol and 1H-Pyrazole-1-carboxamidine.HCl were purchased from Aldrich. All other reagents and solvents were used without further purification. An LNA Oligo-1 targeting survivin gene, Oligo-2 targeting ErbB3 gene and Oligo-3 (scrambled Oligo-2) were prepared in house and their sequences are given in Table 4. The internucleoside linkage is phosphorothioate, ^{m}C represents methylated cytosine, and the upper case letters indicate LNA.

TABLE 4

| LNA Oligo | Sequence |
|---|---|
| Oligo-1 | 5′-^{m}CT^{m}CAatccatgg^{m}CAGc-3′ |
| (SEQ ID NO: 1) | |
| Oligo-2 | 5′-TAGcctgtcactt^{m}CT^{m}C-3′ |
| (SEQ ID NO: 6) | |
| Oligo-3 | 5′-TAGcttgtcccat^{m}CT^{m}C-3 |
| (SEQ ID NO: 25) | |
| Oligo-4 | 5′-gcaugcggccucuguuugadTdT-3′ |
| (SEQ ID NOs: | 3′-dTdTcguacgccggagacaaacu-5′ |
| 2 and 3) | |

Following abbreviations are used throughout the examples such as, LNA (Locked nucleic acid), BACC (2-[N,N′-di(2-guanidiniumpropyl)]aminoethyl-cholesteryl-carbonate), Chol (cholesterol), DIEA (diisopropylethylamine), DMAP (4-N,N-dimethylamino-pyridine), DOPE (L-α-dioleoyl phosphatidylethanolamine, Avanti Polar Lipids, USA or NOF, Japan), DLS (Dynamic Light Scattering), DSPC (1,2-distearoyl-sn-glycero-3-phosphocholine) (NOF, Japan), DSPE-PEG (1,2-distearoyl-sn-glycero-3-phosphoethanolamine-N-(polyethylene glycol)2000 ammonium salt or sodium salt, Avanti Polar Lipids, USA and NOF, Japan), KD (knowndown), EPC (egg phosphatidylcholine, Avanti Polar Lipids, USA) and C16 mPEG-Ceramide (N-palmitoyl-sphingosine-1-succinyl(methoxypolyethylene glycol)2000, Avanti Polar Lipids, USA). Other abbreviations such as the FAM (6-carboxyfluorescein), FBS (fetal bovine serum), GAPDH (glyceraldehyde-3-phosphate dehydrogenase), DMEM (Dulbecco's Modified Eagle's Medium), MEM (Modified Eagle's Medium), TEAA (tetraethylammonium acetate), TFA (trifluoroacetic acid), RT-qPCR (reverse transcription-quantitative polymerase chain reaction) were also used.

### Example 1

### General NMR Method

¹H NMR spectra were obtained at 300 MHz and ¹³C NMR spectra at 75.46 MHz using a Varian Mercury 300 NMR spectrometer and deuterated chloroform as the solvents unless otherwise specified. Chemical shifts (d) are reported in parts per million (ppm) downfield from tetramethylsilane (TMS).

### Example 2

### General HPLC Method

The reaction mixtures and the purity of intermediates and final products are monitored by a Beckman Coulter System Gold® HPLC instrument. It employs a ZORBAX® 300SB C8 reversed phase column (150×4.6 mm) or a Phenomenex Jupiter® 300A C18 reversed phase column (150×4.6 mm) with a 168 Diode Array UV Detector, using a gradient of 10-90% of acetonitrile in 0.05% TFA at a flow rate of 1 mL/minute or a gradient of 25-35% acetonitrile in 50 mM TEAA buffer at a flow rate of 1 mL/minute. The anion exchange chromatography was run on AKTA explorer 100A from GE healthcare (Amersham Biosciences) using Poros 50HQ strong anion exchange resin from Applied Biosystems packed in an AP-Empty glass column from Waters. Desalting was achieved by using HiPrep 26/10 desalting columns from Amersham Biosciences. (for PEG-Oligo)

### Example 3

### General mRNA Down-Regulation Procedure

The cells were maintained in complete medium (F-12K or DMEM, supplemented with 10% FBS). A 12 well plate containing 2.5×10⁵ cells in each well was incubated overnight at 37° C. Cells were washed once with Opti-MEM® and 400 μL of Opti-MEM® was added per each well. Then, a solution of nanoparticles or Lipofectamine2000 containing oligonucleotides was added to each well. The cells were incubated for 4 hours, followed by addition of 600 μL of media per well, and incubation for 24 hours. After 24 hours of treatment, the intracellular mRNA levels of the target gene, such as human ErbB3, and a housekeeping gene, such as GAPDH were quantified by RT-qPCR. The expression levels of mRNA were normalized.

### Example 4

### General RNA Preparation Procedure

For in vitro mRNA down-regulation studies, total RNA was prepared using RNAqueous Kit® (Ambion) following the manufacturer's instruction. The RNA concentrations were determined by OD_{260 nm} using Nanodrop.

### Example 5

### General RT-qPCR Procedure

All the reagents were from Applied Biosystems: High Capacity cDNA Reverse Transcription Kit® (4368813), 20×PCR master mix (4304437), and TaqMan® Gene Expression Assays kits for human GAPDH (Cat. #0612177) and survivin (BIRKS Hs00153353). 2.0 μg of total RNA was used for cDNA synthesis in a final volume of 50 μL. The reaction was conducted in a PCR thermocycler at 25° C. for 10 minutes, 37° C. for 120 minutes, 85° C. for 5 seconds and then stored at 4° C. Real-time PCR was conducted with the program of 50° C.-2 minutes, 95° C.-10 minutes, and 95° C.-15 seconds/60° C.-1 minute for 40 cycles. For each qPCR reaction, 1 μL of cDNA was used in a final volume of 30 μL.

### Example 6

### Preparation of H-Dap-OMe:2HCl (Compound 1)

H-Dap-(Boc)-OMe:HCl (5 g, 19.63 mmol) was treated with 2M HCl in 1,4-dioxane (130 mL) for 30 minutes at room temperature. The solvents were removed in vacuo at 30-35° C. The residue was resuspended in diethyl ether and filtered. Isolated solids were dried in vacuo over P₂O₅ to yield 3.4 g (90%) of product: ¹³C NMR (DMSO-d₆) δ 40.05, 49.98, 53.47, 166.73.

### Example 7

### Preparation of Dioleoyl-Dap-OMe (Compound 2)

A solution of compound 1 (3.4 g, 17.8 mmol) in 26 mL of anhydrous DMF was added to a solution of oleic acid (22.5 mL, 20.0 g, 71.1 mmol) in 170 mL of anhydrous DCM. The mixture was cooled to 0 to 5° C., followed by addition of EDC (20.5 g, 106.7 mmol) and DMAP (28.2 g, 231.1 mmol). The reaction mixture was stirred overnight and warmed to room temperature under nitrogen. Completion of reaction was monitored by TLC (DCM:MeOH=90:1, v/v). The reaction mixture was diluted with 200 mL of reagent grade of DCM and washed with 1N HCl (3×80 mL) and 0.5% aqueous NaHCO₃ (3×80 mL). The resulting organic layer was separated, dried over anhydrous magnesium sulfate and concentrated in vacuo at 30° C. The residue was purified by silica gel column chromatography (DCM/MeOH/TEA=95:5:0.1, v/v/v) to yield 7.0 g (61%) of product: ¹³C NMR δ 14.15, 22.60, 25.55, 25.69, 27.20, 27.25, 29.18, 29.23, 29.29, 29.34, 29.55, 29.75, 29.78, 31.91, 36.43, 36.52, 41.53, 52.63, 53.58, 129.49, 129.54, 129.82, 129.85, 170.55, 173.59, 174.49.

### Example 8

### Preparation of Dioleoyl-Dap-OH (Compound 3)

A solution of NaOH (0.87 g, 21.63 mmol) in 7 mL of water was added to a solution of compound 2 (7.0 g, 10.8 mmol) in 70 mL of ethanol. The mixture was stirred at room temperature overnight and concentrated in vacuo at room temperature. The residue was suspended in 63 mL of water and the solution was acidified with 1N HCl at 0 to 5° C. The aqueous solution was extracted with DCM three times. Organic layers were combined and dried over anhydrous magnesium sulfate. The solvent was removed in vacuo at 35° C. to yield 5.5 g (80%) of product: ¹³C NMR δ 14.19, 22.75, 25.51, 25.68, 27.25, 27.29, 29.21, 29.26, 29.32, 29.38, 29.59, 29.79, 29.82, 31.95, 36.30, 36.37, 41.58, 55.15, 129.53, 129.91, 171.49, 175.67, 176.19.

### Example 9

### Preparation of Compound 5

N-(2-hydroxyethyl)phthalimide (4, 25 g, 130.8 mmol, 1 eq.) was dissolved in 500 mL of dry benzene and azeotroped for 1 hour, removing 125 mL of benzene, followed by cooling to room temperature and addition of p-TsOH (0.240 g, 1.26 mmol, 0.0096 eq). The mixture was cooled to 0-5° C., then added 2-methoxypropene (10.4 g, 13.8 mL, 143.8 mmol, 1.1 eq.) through an addition funnel over 15 minutes at 0-5° C. The reaction mixture was stirred at 0-5° C. for 1 hour, followed by heating to 89-95° C. and azeotroping for 3 hours to remove MeOH/benzene. Following each removal of solvent, the solution was cooled to stop the azeotroping and an equivalent volume of benzene was added. After 3 hours, the reaction mixture was cooled to room temperature and added 30 mL of TEA and 5 mL of acetic anhydride and stirred overnight at room temperature. The reaction mixture was concentrated in vacuo at 35° C. to remove ⅔ volume of benzene and crude products were precipitated with 300 mL of hexane dropwise. The precipitates were filtered and washed with hexane. The solids (8.5 g) were dissolved in 70 mL of toluene at 65° C. and the solution was cooled to 0° C. The product was collected by centrifugation, washed with hexane, and coevaporated with CCl₄ in vacuo to yield 4.9 g of product: ¹³C NMR δ 24.67, 38.09, 57.88, 100.39, 123.05, 131.92, 133.66, 167.88.

### Example 10

### Preparation of Compound 6

Compound 5 (4.9 g, 11.6 mmol) was dissolved in 6 M NaOH (9.1 g of NaOH in 38 mL water) and the solution was refluxed overnight. The resulting solution was cooled to room temperature, then extracted three times with 40 mL of 1:1 (v/v) of chloroform/IPA, dried over anhydrous sodium sulfate, and concentrated in vacuo at 35° C. The solids were suspended in hexane twice and once in CCl₄, and dried in vacuo at 35° C. to obtain the product (1.8 g, 95%): ¹³C NMR δ 24.99, 42.08, 43.81, 62.82, 63.58, 77.41, 99.64.

### Example 11

### Preparation of Compound 7

Compound 6 (1.8 g, 11.1 mmol, 1 eq.) was dissolved in 36 mL of anhydrous THF, cooled to −78° C. in a dry ice/IPA bath, followed by addition of ethyl-trifluoroacetate. The reaction mixture was stirred at room temperature for 1.5 hours before the solvent was removed in vacuo by coevaporating with hexane to give crude product. The crude product was purified by column chromatography on deactivated alumina using DCM and MeOH (100:0.1 to 98:2, v/v) to yield 1.30 g of product: ¹³C NMR δ 24.88, 40.68, 41.11, 42.13, 57.99, 60.26, 62.10, 99.83.

### Example 12

### Preparation of Compound 8 (MW 2,000)

mPEG-OH (MW 2,000, 50 g) was recrystallized from 500 mL IPA at 65° C. to obtain 44 g of dried mPEG-OH. The recrystallized mPEG-OH (44 g, 22 mmol, 1 eq.) was dissolved in 775 mL of anhydrous DCM. Triphosgene (2.61 g, 8.8 mmol, 0.40 eq) and pyridine (2.1 mL, 2.1 g, 26.4 mmol, 1.20 eq) were added to the solution and the reaction mixture was stirred for 4 hours at room temperature. To the resulting reaction solution, NHS (3.4 g, 29.3 mmol, 1.33 eq) and pyridine (2.4 mL, 2.3 g, 29.3 mmol, 1.33 eq.) were added and the mixture was stirred overnight at room temperature. The reaction mixture was concentrated in vacuo and the residue was dissolved in 88 mL of DCM. Addition of ether precipitated solids which were recrystallized from a mixture of 44 mL acetonitrile/1600 mL IPA. The solids were filtered, washed with IPA and ether, and dried in vacuo to give SCmPEG. SCmPEG (MW 2,000, 5.76 g, 2.88 mmol, 1 eq.) and compound 7 (1.30 g, 5.0 mmol, 1.75 eq) were dissolved in 60 mL dry DCM and 8 mL dry DMF. DIEA (0.60 g, 0.82 mL. 4.61 mmol, 1.6 eq) was added and the reaction mixture was stirred at room temperature overnight. The resulting reaction solution was concentrated in vacuo at room temperature, followed by addition of ether to precipitate solids at 0-5° C. in an ice bath. The solids were collected by centrifugation and recrystallized from a mixture of 2 mL acetonitrile and 80 mL IPA. The product was collected by centrifugation and washed with IPA and ether, dried in vacuum oven at 40° C. to yield 5.5 g, 90% of product: ¹³C NMR δ 24.72, 39.80, 40.95, 58.45, 58.73, 58.96, 59.74, 63.86, 69.49, 70.06, 70.45, 70.77, 71.83, 76.21, 77.20, 100.20, 113.80, 117.60, 156.25, 157.26.

### Example 13

### Preparation of Compound 9

A solution of potassium carbonate (0.393 g, 2.84 mmol, 1.1 eq.) in 7 mL of water was added to a solution of compound 8 (5.5 g, 2.59 mmol, 1 eq.) in 44 mL reagent grade MeOH. The reaction solution was stirred overnight at room temperature, followed by removal of MeOH in vacuo. The residue was dissolved in 500 mL DCM, washed with 25 mL water, with 35 mL brine, dried over anhydrous magnesium sulfate, filtered and concentrated in vacuo at room temperature. The residue was recrystallized from a mixture of 2.5 mL acetonitrile and 80 mL IPA. The product was collected by centrifugation and washed with IPA and ether, dried in vacuum oven at 40° C. to yield 3.38 g of product: ¹³C NMR δ 24.93, 25.38, 41.22, 41.98, 59.00, 59.57, 62.97, 63.83, 69.61, 70.10, 70.50, 71.87, 75.78, 76.19, 77.20, 99.79, 156.27.

### Example 14

### Preparation of Compound 10

Compound 9 (20 mmol) was dissolved in 50 mL of anhydrous DMF and 400 mL of anhydrous DCM and the solution was cooled in an ice bath. DMAP (6.2 g, 51.2 mmol) was added to the solution, followed by addition of compound 3 (40 mmol) and EDC (40 mmol). The solvent was removed and the residue was recrystallized from DCM/ethyl ether twice to give the product.

### Example 15

### Preparation of BocNHCH₂CH₂NH₂ (Compound 11)

A solution of Boc-anhydride (60 g, 274.9 mmol) in 150 mL of anhydrous DCM was slowly added to a solution of ethane-1,2-diamine (41.3 g, 687.3 mmol) in 250 mL of anhydrous THF and 200 mL of anhydrous DCM at 0-5° C. over 1.5 hours. The reaction mixture was stirred overnight while warmed to room temperature. 300 mL of water was added to the mixture, which was concentrated under vacuum at 30° C. The resulting aqueous solution was washed with DCM (3×300 mL) and the organic layers were combined and extracted with 0.5 N HCl (3×300 mL). Aqueous layers were combined and pH was adjusted to 9-10 with 4N NaOH solution, followed by extraction with DCM (3×500 mL). Organic layers were combined and dried over anhydrous magnesium sulfate. The solvent was removed in vacuo at 35° C. to yield 17.6 g (40%) of product: ¹³C NMR δ 28.23, 41.67, 43.19, 78.77, 155.93.

### Example 16

### Preparation of Dioleoyl-Dap-NHCH₂CH₂NHBoc (Compound 12)

DMAP (6.2 g, 51.2 mmol) was added to a solution of compound 3 (5.4 g, 8.53 mmol) in 50 mL of anhydrous DMF and 400 mL of anhydrous DCM and the solution was cooled in an ice bath. Compound 11 (2.73 g, 17.1 mmol) and EDC (6.6 g, 34.1 mmol) were added to the solution and the solution was stirred overnight while allowed to warm to room temperature. Completion of reaction was monitored by TLC (DCM/MeOH=9:1, v/v) and the reaction mixture was diluted with 500 mL of DCM, washed with 0.2 N HCl (3×500 mL) and water (3×500 mL), and dried over anhydrous magnesium sulfate. The solvent was removed in vacuo at 35° C. to yield 5.6 g (85%) of product: ¹³C NMR δ 14.16, 22.72, 25.52, 25.77, 27.23, 27.26, 28.43, 29.24, 29.35, 29.56, 29.79, 31.92, 36.50, 40.25, 40.38, 41.99, 55.22, 76.57-77.42 (CDCl₃), 79.41, 129.54, 129.86, 156.35, 170.44, 174.25, 175.35.

### Example 17

### Preparation of Dioleoyl-Dap-NHCH₂CH₂NH₂ (Compound 13)

Compound 12 (5.6 g, 7.2 mmol) was dissolved in 95 mL DCM and the solution was treated with 24 mL of trifluoroacetic acid for 30 minutes at room temperature. The solvent was removed in vacuo at room temperature and the residue was redissolved in 200 mL DCM. The solution was washed with water and with 1% NaHCO₃ several times until pH was 8-9. Organic layer was dried over anhydrous magnesium sulfate and the solvent was removed in vacuo at 30° C. to yield 4.13 g (85%) of product: ¹³C NMR δ 14.15, 22.70, 25.62, 25.77, 27.25, 29.24, 29.35, 29.55, 29.78, 31.91, 36.43, 41.53, 54.95, 129.48, 129.85, 170.99, 174.43, 175.33.

### Example 18

### Preparation of 4-(dimethyl acetal) benzoic acid (Compound 14)

4-Formyl benzoic acid (1.5 g, 10 mmol) was dissolved in 30 mL of anhydrous methanol followed by the addition of 1.0 M lithium tetrafluororoborate in acetonitrile (300 μL, 0.3 mmol), trimethyl orthoformate (1.38 g, 10 mmol). The reaction mixture was refluxed overnight. The solvent was removed and the residue was suspended in boiling hexane for 30 minutes. The mixture was cooled to room temperature and the solid was isolated by filtration to yield 1.5 g (77%) of product: ¹³C NMR (CD₃OD) δ 53.26, 103.88, 127.75, 130.47, 131.14, 144.29, 169.30.

### Example 19

### Preparation of 4-(dimethyl acetal)phenylcarboxyamino PEG (Compound 15)

mPEG-amine (MW 5,000, 3 g, 0.60 mmol) and DMAP (219.6 mg, 1.80 mmol) were dissolved in 30 mL of anhydrous DCM. The mixture was cooled to 0-5° C., followed by the addition of EDC (345.6 mg, 1.80 mmol) and compound 14 (352.8 mg, 1.80 mmol). The reaction mixture was stirred at 0° C. to room temperature overnight under N₂. The solvent was removed and the residue was recrystallized from mixed solvent of DMF/IPA (10 mL/100 mL) to give 2.7 g (82%) of product: ¹³C NMR δ 39.60, 52.38, 58.79, 69.63-71.67 (PEG), 102.06 [—C(OMe)₂], 126.50, 126.7, 134.30, 140.90, 166.72.

### Example 20

### Preparation of 4-formylphenylcarboxyamino PEG (Compound 16)

Compound 15 (2.4 g, 0.46 mmol) in 6.75 mL chloroform was treated with 1.68 mL of 86% formic acid at room temperature overnight. The solvent was removed and the residue was recrystallized from DCM ethyl ether twice to give the product (2.3 g, 97%): ¹³C NMR δ 39.82, 58.79, 69.34-71.67 (PEG), 127.59, 129.34, 137.69, 139.43, 165.91, 191.21 (HC═O).

### Example 21

### Preparation of 4-Dioleoyl-Dap-NHCH₂CH₂-iminomethylphenylcarboxamino-PEG (Compound 17)

Compound 13 (202.5 mg, 0.30 mmol) was dissolved in 10 mL of anhydrous DCM and 2 mL of anhydrous DMF, followed by addition of compound 16 (1.0 g, 0.2 mmol), molecular sieves (2 g) and DIEA (25.8 mg, 0.2 mmol). The reaction mixture was stirred at room temperature overnight under N₂. The reaction mixture was filtered and the filtrate was concentrated in vacuo. The residue was recrystallized from acetonitrile-IPA. The very fine solid was isolated by centrifugation to give 0.6 g (52%) of product: ¹³C NMR δ 13.94, 22.20, 22.45, 25.42, 25.61, 26.96, 28.96, 29.07, 29.27, 29.51, 31.65, 36.17, 36.46, 38.20, 39.66, 39.82, 52.65, 58.73, 59.92, 69.40-71.64 (PEG), 127.11, 127.78, 129.30, 129.54, 136.20, 137.97, 161.44 (—C═N—), 166.45, 171.49, 173.01.

### Example 22

### Preparation of Dioleoyl-Lys-Ethyl Ester (Compound 18)

L-Lysine-ethyl ester (2.1 g, 8.55 mmol) and oleic acid (14.5 g, 51.3 mmol) were dissolved in 105 mL of anhydrous DCM and the solution was cooled in an ice bath. EDC (9.9 g, 51.3 mmol) was added to the solution, followed by addition of DMAP (15.5 g, 127.4 mmol). The reaction mixture was stirred overnight at 0° C. to room temperature. The reaction mixture was washed with dilute HCl until a pH was adjusted to 2. Crude product was purified by silica gel column chromatography using 3.5% MeOH in DCM to yield 3.9 g (65%) of product: ¹³C NMR: δ 13.97, 14.03, 22.23, 22.53, 25.54, 25.72, 27.04, 28.37, 28.78, 29.04, 29.16, 29.37, 29.59, 31.59, 31.74, 36.21, 36.50, 38.42, 51.67, 53.25, 61.03, 129.33, 129.59, 172.14, 172.96, 173.18.

### Example 23

### Preparation of Dioleoyl-Lys-OH (Compound 19)

A solution of NaOH (0.393 g, 9.84 mmol) in 3.5 mL of water was added to a solution of compound 18 (3.46 g, 4.92 mmol) in 32 mL of ethanol. The reaction mixture was stirred at room temperature overnight and cooled to 0-5° C. 20 mL of 0.5N HCl (ice cold) was added to the reaction mixture to obtain pH 2.5, followed by extraction with DCM (3×100 mL). Organic layers were combined and dried over magnesium sulfate and solvent was removed to yield 3.23 g (97%) of product: ¹³C NMR: δ 14.12, 22.19, 22.68, 25.72, 25.86, 27.22, 28.83, 29.20, 29.32, 29.52, 29.75, 31.61, 31.89, 36.43, 36.66, 38.95, 51.96, 129.51, 129.82, 173.92, 174.17, 174.27.

### Example 24

### Preparation of Dioleoyl-Lys-NHCH₂CH₂NHBoc (Compound 20)

Compound 19 (2.62 g, 3.88 mmol) was dissolved in 75 mL of anhydrous DMF and 200 mL of anhydrous DCM and the solution was cooled to 0-5° C., followed by addition of DMAP (2.84 g, 23.29 mmol), Compound 11 (1.24 g, 7.76 mmol) and EDC (2.98 g, 15.53 mmol). The reaction mixture was stirred overnight under nitrogen 0° C. to room temperature. Completion of reaction was monitored by TLC (DCM/MeOH=9:1, v/v). The reaction mixture was diluted with 250 mL of DCM, washed with 0.2N HCl (3×250 mL) and water (3×200 mL). Organic layer was dried over anhydrous magnesium sulfate and the solvent was removed to yield 2.81 g (89%) of product: ¹³C NMR: δ 14.15, 22.50, 22.70, 25.70, 25.89, 27.22, 27.25, 28.44, 29.07, 29.23, 29.34, 29.53, 29.78, 31.91, 32.02, 36.52, 36.78, 38.68, 40.13, 52.80, 79.36, 129.53, 129.83, 156.36, 172.15, 173.39.

### Example 25

### Preparation of Dioleoyl-Lys-NHCH₂CH₂NH₂ (Compound 21)

Compound 20 (2.82 g, 3.45 mmol) was dissolved in 48 mL of reagent grade DCM, followed by addition of 12 mL of trifluoroacetic acid. The reaction mixture was stirred for 30 minutes at room temperature followed by concentrated in vacuo at room temperature. Oily residue was redissolved in 100 mL of DCM and washed with 1% aqueous NaHCO₃ solution until pH was 8-9. Organic layer was dried over anhydrous magnesium sulfate and the solvent was removed to yield 1.96 g (80%) of product: ¹³C NMR: δ 14.18, 22.52, 22.73, 25.77, 25.89, 27.23, 27.26, 29.15, 29.23, 29.35, 29.56, 29.78, 31.81, 31.92, 36.55, 36.84, 38.59, 52.98, 129.54, 129.86, 172.05, 173.38, 173.53.

### Example 26

### Preparation of 4-Dioleoyl-Lys-NHCH₂CH₂-iminomethylphenylcarboxamino-PEG (Compound 22)

Compound 21 (286.8 mg, 0.40 mmol) was dissolved in 10 mL of DCM and 2 mL of DMF, followed by addition of compound 16 (1.0 g, 0.2 mmol), molecular sieves (2 g) and DIEA (25.8 mg, 0.2 mmol). The reaction mixture was stirred at room temperature overnight under N₂ and filtered. The solvent was removed in vacuo and the residue was recrystallized from acetonitrile-IPA. The very fine solid was isolated by centrifugation to give 0.6 g (52%) of product: ¹³C NMR δ 13.94, 22.20, 22.45, 25.42, 25.61, 26.96, 28.96, 29.07, 29.27, 29.51, 31.65, 36.17, 36.46, 38.20, 39.66, 39.82, 52.65, 58.73, 59.92, 69.40-71.64 (PEG), 127.11, 127.78, 129.30, 129.54, 136.20, 137.97, 161.44 (—C═N—), 166.45, 171.49, 173.01.

### Example 27

### Preparation of Compound 25

mPEG-Tosylate (MW 2,000, compound 23, 3 g, 1.39 mmol), 2-methoxy 4-hydroxy benzaldehyde (compound 24, 52.9 mg, 3.48 mmol, 2.5 eq) and potassium carbonate (576.6 mg, 4.18 mmol, 3 eq) in anhydrous DMF were stirred at 60-65° C. overnight. After completion of the reaction was confirmed by HPLC, the mixture was cooled to room temperature and filtered. Ethyl ether (300 mL) was added to precipitate crude product. The crude produce was filtered and the isolated wet cake was dissolved in DCM (100 mL) and washed with 0.5% NaHCO₃ (2×10 mL). The organic layer was dried over anhydrous MgSO₄ and concentrated to dryness. The residue was recrystallized from CH₃CN/IPA (2 mL/80 mL). The precipitate was isolated by filtration and dried under vacuum at 35° C. to yield 1.75 g of the product: ¹³C NMR δ 55.6, 58.9, 67.7-71.8 (PEG), 98.5, 106.0, 118.9, 130.5, 163.3, 165.1, 187.9.

### Example 28

### Preparation of Compound 26

Compound 25 (868 mg, 0.41 mmol) and compound 13 (480 mg, 0.71 mmol, 1.75 eq) were dissolved in a mixture of DCM (15 mL) and DMF (2 mL). Molecular sieves (2 g) were added, followed by addition of DIEA (52.5 mg, 0.41 mmol, 1.0 eq). The mixture was stirred at room temperature overnight and filtered. The filtrate was concentrated and the residue was precipitated with ethyl ether and centrifuged. Isolated wet solids were recrystallized from CH₃CN/IPA. The solids were isolated by centrifugation and dried under vacuum at 35° C. to yield 570 mg of product: ¹³C NMR δ 14.1, 22.7, 25.4, 25.7, 27.2, 29.2, 29.3, 29.5, 29.7, 31.9, 36.4, 36.5, 40.4, 42.1, 55.2, 55.4, 59.0, 60.2, 67.4-76.6 (PEG), 98.6, 105.8, 117.6, 128.3, 129.5, 129.8, 158.0 159.9, 162.2, 170.0, 174.1, 175.2.

### Example 29

### Preparation of Compound 29

Boc-NHCH₂CH₂NH₂ (11, 4 g, 25.0 mmol, 1.2 eq.) was reacted with 4-methoxy benzoyl chloride (27, 3.6 g, 20.81 mmol, 1 eq.) in the presence of TEA (4.27 g, 5.9 mL, 42.2 mmol, 2 eq.) in 35 mL anhydrous THF for 30 minutes at room temperature. Reaction completion was checked by TLC. The reaction mixture was diluted with 350 mL DCM, washed with 300 mL 1N HCl, and 300 mL water, dried over anhydrous magnesium sulfate, and concentrated in vacuo to yield 7.2 g, 98% of product: ¹³C NMR δ 28.38, 40.10, 41.81, 55.33, 79.70, 113.49, 126.37, 128.71, 157.26, 161.90, 167.27.

### Example 30

### Preparation of Compound 30

Boc-NHCH₂CH₂NHCO-4-methoxy benzene (compound 29, 7.1 g, 24.1 mmol) was dissolved in 23 mL of DCM:TFA (4:1, v/v) and stirred at room temperature for 30 minutes. The reaction completion was checked by TLC. The solvents were removed in vacuo at room temperature and the residue was dissolved in 40 mL DCM, washed once with 40 mL 1 N NaOH and the organic layer was dried over anhydrous magnesium sulfate. The solvent was removed in vacuo to yield 2.65 g, 57% of product: ¹³C NMR (DMSO-d₆) δ 41.32, 42.54, 55.10, 113.23, 126.57, 128.48, 161.58, 167.08.

### Example 31

### Preparation of Compound 31

Compound 3 (3.88 mmol) was dissolved in 75 mL of anhydrous DMF and 200 mL of anhydrous DCM and the solution was cooled to 0-5° C., followed by addition of DMAP (2.84 g, 23.29 mmol), compound 7 (7.76 mmol) and EDC (2.98 g, 15.53 mmol). The reaction mixture was stirred overnight under nitrogen from 0° C. to room temperature. The reaction mixture was diluted with 250 mL of DCM, washed with 0.2N HCl (3×250 mL) and water (3×200 mL). Organic layer was dried over anhydrous magnesium sulfate and the solvent was removed to yield product.

### Example 32

### Preparation of Compound 32

Compound 31 (0.102 mmol) was treated with K₂CO₃ (42 mg, 0.305 mmol) in CH₃OH/H₂O at room temperature. The reaction was followed by HPLC. After reaction was completed, the solvent was removed and the residue was redissolved in DCM and filtered through 0.45 um membrane. The solvent was removed and the residue was recrystallized from IPA to yield the product:

### Example 33

### Preparation of Compound 34

HO-^{2K}PEG-COOH (33, 7 g, 3.5 mmol, 1 eq.) was dissolved in anhydrous MeOH (56 g, 70.8 mL, 1750 mmol, 500 eq.) and 70 mL anhydrous DCM. The mixture was cooled to 0° C., followed by addition of EDC (3.36 g, 17.5 mmol, 5 eq.), and DMAP (2.1 g, 17.5 mmol, 5 eq) at 0° C. The reaction mixture was stirred overnight at room temperature, and concentrated in vacuo. The residue was redissolved in 40 mL of 0.1 N HCl (pH ˜2), and extracted three times with DCM. The organic layers were combined and dried over anhydrous magnesium sulfate and the solvent was removed in vacuo. The residue was recrystallized from 100 mL IPA, recovered and washed with ether by centrifugation and dried in vacuo at 40° C. to yield 6.5 g (92%) of product: ¹³C NMR δ 51.77, 61.70, 68.57, 70.36, 70.50, 70.85, 72.42, 170.65.

### Example 34

### Preparation of Compound 35

HO-2 kPEG-COOMe (34, 6.3 g, 3.15 mmol, 1 eq.) and DMAP (1.92 g, 15.75 mmol, 5 eq) were dissolved in 38 mL anhydrous DCM and cooled to 0 degrees. Tosyl chloride (3.00 g, 15.75 mmol, 5 eq) in 63 ml anhydrous DCM was added dropwise over 3 hours at 0° C. The mixture was stirred overnight at 0 degrees to room temperature The solvent was removed and residue was precipitated with IPA to yield 5.85 g product: ¹³C NMR δ 21.73, 51.80, 68.61, 69.22, 70.53, 70.88, 76.21, 77.21, 127.86, 129.69, 132.82, 144.62, 170.69.

### Example 35

### Preparation of Compound 36

NaOH (0.066 g, 1.65 mmol, 1.1 eq.) was added to a solution of TsO-PEG-COOMe (compound 35, 3.00 g, 1.5 mmol, 1 eq.) in 15 mL water. The reaction monitored by HPLC-ELSD was completed after 3 hours. The solution was acidified to pH 2 with addition of 1 HCl dropwise at 0° C. and extracted with 150 mL DCM three times. The organic layers were combined, dried over anhydrous magnesium sulfate and concentrated in vacuo at 30° C. The residue was recrystallized from 20 mL IPA and isolated with centrifugation. The final product was dried in vacuo at 40° C. to yield 2.6 g product: ¹³C NMR δ 21.67, 68.59, 68.79, 69.17, 70.30, 70.45, 71.22, 127.80, 128.65, 132.81, 144.58, 171.29.

### Example 36

### Preparation of Compound 37

EDC (2.84 mmol) and DMAP (5.68 mmol) were added to a solution of TsO-PEG-COOH (36, 2.0 g, 1.00 mmol, 1 eq.) and compound 32 (2.42 mmol) in 20 mL anhydrous DCM and 4 mL DMF at 0° C. The reaction mixture was stirred overnight at 0° C. to room temperature. The solution was concentrated in vacuo at room temperature and the residue was precipitated with ether and isolated with centrifugation. The material was purified by alumina (deactivated, 3% water) column chromatography with 0-2% MeOH in DCM, v/v, to yield 1.16 g of product: ¹³C NMR δ 14.10, 22.61, 24.74, 25.51, 25.64, 27.13, 29.10, 29.22, 29.45, 29.68, 31.82, 36.43, 38.86, 39.45, 40.17, 41.71, 54.55, 59.22, 59.34, 68.53, 69.11, 70.42, 99.77, 127.74, 129.46, 129.57, 129.72, 169.99, 174.11, 174.86.

### Example 37

### Preparation of Compound 38

Compound 30 (5 eq.) and Et₃N (5 eq.) were added to a solution of TsO-PEG-COO-Dap-lipid (37, 1.0 eq.) in DMSO (2 vol) at room temperature. The reaction was heated at 90° C. for 2.5 hours. The material was recrystallized from IPA at −78° C., washed with Et₂O twice, dried at 40° C. under vacuum, and purified by neutral alumina column chromatography to give product in 60% yield: ¹³C NMR δ 14.13, 22.66, 24.78, 25.54, 25.68, 27.16, 28.41, 29.15, 29.48, 29.71, 31.86, 38.49, 38.89, 39.25, 39.48, 41.80, 48.24, 28.55, 54.58, 55.29, 59.26, 59.39, 70.25, 70.39, 70.42, 70.88, 99.82, 113.42, 126.76, 128.49, 129.49, 129.78, 161.76, 166.77, 170.02, 174.16, 174.94.

### Example 38

### Preparation of Compound 39

A solution of compound 30 (1 g, 5.15 mmol), BocNHCH₂CH₂Br (35, 1.38 g, 6.18 mmol) and DIPEA (1.33 g, 10.3 mmol) were refluxed in THF (20 ml). The reaction was monitored by by TLC. After reaction is completed, the solvent was removed and the residue was purified by silica gel column to yield 0.78 g, 28% of product: ¹³C NMR δ 28.25, 39.25, 39.98, 48.27, 48.62, 55.15, 78.92, 113.31, 126.37, 128.63, 156.00, 161.70, 167.11.

### Example 39

### Preparation of Compound 40

Ethyl trifluoroethanoate (0.42 g, 2 mmol) was added slowly to a mixture of tert-butyl 2-(2-(4-methoxybenzamido)ethylamino)ethylcarbamate (39, 0.45 g, 1.33 mmol) and DIEA (0.52 g, 4 mmol) in THF (20 ml) and the mixture was stirred for 15 min at −10˜−15° C. 50 ml of brine was added to quench the reaction and the solution was extracted with ethyl acetate several times. The organic layers were combined and dried over anhydrous MgSO₄. The solvent was removed and the residue was purified by silica gel column chromatography to yield 0.52 g, 90% of product: ¹³C NMR δ 27.171, 28.02, 30.10, 37.54, 38.01, 38.42, 44.52, 45.27, 45.59, 46.76, 47.59, 48.12, 55.06, 55.41, 55.46, 60.15, 79.32, 113.27, 113.37, 113.90, 114.33, 114.42, 117.13, 117.71, 118.00, 122.58, 122.67, 125.15, 125.88, 125.95, 128.55, 128.77, 131.97, 132.12, 155.63, 155.23, 157.04, 159.58, 160.10, 161.88, 164.52, 164.81, 167.27, 170.24, 170.53, 170.82.

### Example 40

### Preparation of Compound 41

TFA (2 ml) was added to a solution of tert-butyl 2-(2,2,2-trifluoro-N-(2-(4-methoxybenzamido)ethyl)acetamido)ethylcarbamate (40, 0.2 g) in DCM (8 ml). The mixture was stirred at room temperature and the reaction was monitored by TLC. After reaction was completed, the solvent was removed in vacuo to yield 100% product: ¹³C NMR (CD₃OD) δ 37.37, 37.78, 37.92, 38.45, 38.85, 46.15, 46.50, 47.78, 47.89, 48.15, 55.86, 11466, 115.11, 118.94, 119.43, 126.86, 127.04, 12/9.02, 129.75, 130.00, 130.29, 159.24, 159.71, 160.83, 161.33, 163.83, 163.989, 164.04, 170.05, 170.17, 170.86.

### Example 41

### Preparation of Compound 42

A mixture of TFA salt of N-(2-(N-(2-aminoethyl)-2,2,2-trifluoroacetamido)ethyl)-4-methoxybenzamide (41, 255 mg, 0.593 mmol), succinic anhydride (59 mg, 0.593 mmol) and TEA (59 mg, 0.593 mmol) in DCM (10 ml) was stirred at room temperature. The reaction progress was followed by TLC. After the reaction was completed, the solvent was removed and the residue was purified with silica gel column chromatography to yield 170 mg, 66% of product: ¹³C NMR (CD₃OD) δ 28.84, 30.09, 30.31, 38.75, 38.89, 39.10, 46.07, 46.70, 48.15, 55.91, 114.63, 116.07, 119.12, 119.96, 127.05, 127.28, 129.97, 130.00, 158.52, 159.01, 162.28, 162.73, 163.57, 163.66, 169.58, 174.53, 174.61, 175.92, 176.11.

### Example 42

### Preparation of Compound 43

EDC (151 mg, 0.786 mmol) was added to a mixture of 4-oxo-4-(2-(2,2,2-trifluoro-N-(2-(4-methoxybenzamido)ethyl)acetamido)ethylamino)butanoic acid (42, 170 mg, 0.393 mmol), NHS (91 mg, 0.786 mmol) and DMAP (144 mg, 1.18 mmol) in DCM in an ice bath and the mixture was stirred at 0° C. to room temperature for 3 hours. The reaction mixture was washed with 0.5 N HCl and dried over anhydrous Na₂SO₄. The solvent was removed in vacuo to yield 0.2 g of crude product, which was used without further purification.

### Example 43

### Preparation of Compound 44

A mixture of an activated ester (43, 0.2 g, 0.37 mmol), NH₂—PEG(2,000)-COOH (0.4 g, 0.2 mmol) and DIEA (0.35 ml, 2 mmol) in DCM (10 ml) was stirred at room temperature overnight followed by washing with 1 N HCl. The reaction mixture was dried over anhydrous Na₂SO₄. The solvent was removed in vacuo and the residue was recrystallized from IPA to yield 0.3 g, 62% of product: ¹³C NMR δ 25.16, 27.45, 27.58, 29.31, 30.92, 38.24, 38.38, 38.56, 39.00, 39.08, 45.62, 46.06, 48.01, 55.01, 68.14-70.52 (PEG), 77.20, 113.15, 113.54, 125.61, 125.75, 128.51, 128.64, 156.76, 157.25, 161.61, 161.72, 166.84, 167.07, 169.42, 171.25, 171.81, 171.95, 173.44, 174.02.

### Example 44

### Preparation of Compound 45

EDC was added to a mixture of anisamide-PEG acid (44, 0.3 g, 0.123 mmol), ketal lipid (32, 0.185 g, 0.247 mmol) and DMAP (90 mg, 0.74 mmol) in DCM (20 ml) at 0-5° C. and the reaction mixture was stirred 0° C. to room temperature overnight. The solvent was removed in vacuo and residue was recrystallized from IPA to yield 0.32 g, 82% of product: ¹³C NMR δ 13.83, 22.32, 24.49, 25.10, 25.29, 25.37, 26.85, 27.46, 27.60, 28.80, 28.94, 29.15, 29.31, 30.98, 31.53, 36.08, 36.11, 38.27, 38.38, 38.58, 38.94, 39.03, 39.16, 41.23, 45.62, 46.09, 47.44, 48.03, 54.21, 54.97, 58.95, 59.07, 63.48, 69.22-70.58 (PEG), 99.49, 113.09, 125.72, 125.85, 128.48, 128.63, 129.16, 129.42, 161.55, 161.66, 166.67, 166.98, 169.71, 169.80, 171.46, 171.80, 173.37, 173.75, 173.91, 174.56.

### Example 45

### Preparation of Compound 46

A mixture of the protected anisamide-PEG-ketal lipid (45, 0.32 g 0.102 mmol) and K₂CO₃ (42 mg, 0.305 mmol) in CH₃OH/H₂O was stirred at room temperature until the reaction was completed, monitored by HPLC. The solvent was removed and the residue was redissolved in DCM and filtered through 0.45 μm membrane. The solvent was removed and residue was recrystallized from IPA to yield 0.28 g of product: ¹³C NMR δ 14.15, 22.69, 24.81, 24.84, 25.59, 25.72, 27.20, 27.23, 28.39, 29.17, 29.31, 29.52, 29.69, 29.76, 31.38, 31.90, 36.53, 38.93, 39.31, 39.54, 39.82, 40.54, 41.85, 45.04, 46.05, 48.43, 51, 57, 54.63, 55.32, 59.30, 59.44, 69.71-70.92 (PEG), 77.20, 99.87, 113.46, 126.43, 128.75, 128.95, 129.54, 161.79, 166.81, 167.13, 170.06, 170.15, 171.86, 172.01, 173.88, 174.20, 174.97.

### Example 46

### Preparation of Compound 47

Benzoxy diethyl amine (30, 5 eq.) and TEA (5 eq.) was added to a solution of TsO-PEG-COOMe (35, 1.0 equiv) in DMSO (2 vol) at room temperature and the reaction mixture was stirred at 80° C. for 1.5 hours. The reaction progress was monitored. DCM was added to the reaction mixture and the reaction mixture was washed with water and 0.1N HCl. The organic layer was dried, filtered and concentrated. The residue was recrystallized from IPA, and washed with Et₂O twice, dried at 40° C. in vacuo to give target compound (870 mg) in 88% yield which was confirmed by NMR: ¹³C NMR δ 46.60, 47.57, 48.30, 51.75, 55.31, 65.80, 68.56, 70.83, 113.39, 125.63, 128.75, 129.48, 162.05, 167.40, 170.65.

### Example 47

### Preparation of Compound 48

A mixture of anisamide-PEG-COOMe (48, 1.0 eq.), water (5 vol.) and NaOH (1.1 eq.) was stirred at room temperature overnight. The reaction was monitored by HPLC. DCM was added to the reaction mixture. The reaction mixture was washed with water and 0.1N HCl. The organic layer was dried, filtered and concentrated. The residue was recrystallized from IPA, washed with Et₂O twice, and dried at 40° C. under vacuum to give the product (80 mg) in 95% yield: ¹³C NMR δ 36.63, 47.56, 48.27, 55.31, 65.83, 68.83, 70.01, 70.42, 71.17, 109.67, 113.41, 125.62, 128.59, 129.46, 162.05, 167.44, 171.39.

### Example 48

### Preparation of Compound 49

Boc₂O (69 mg, 1.4 eq.) and TEA (0.044 ml, 1.4 eq.) were added to a solution of anisamide-PEG-COOH (48, 450 mg, 1.0 eq.) in DCM and the reaction mixture was stirred at room temperature for 1 hour. The solution was washed with 0.1N HCl, dried, filtered and concentrated. The residue was recrystallized from IPA, and washed with Et₂O twice, and dried at 40° C. under vacuum to give product (420 mg) in 93% yield: ¹³C NMR δ 28.30, 42.60, 47.80, 55.21, 68.55, 69.85, 70.20, 70.26, 70.40, 71.18, 77.43, 113.58, 161.66, 166.69, 171.22.

### Example 49

### Preparation of Compound 51

DMAP (98 mg, 4 eq.) and EDC (115 mg, 3 eq.) were added to a solution of compound 49 (400 mg, 1.0 eq.) and DSPE-amine (449 mg, 3 eq.) in DCM at 0° C. The mixture was stirred at room temperature overnight. The solution was washed with 0.1N HCl, dried, filtered and concentrated. The residue was recrystallized from IPA, and washed with Et₂O twice, and dried at 40° C. under vacuum to give product (357 mg) in 75% yield: ¹³C NMR δ 13.95, 14.74, 22.47, 24.62, 24.69, 24.75, 28.18, 28.94, 29.31, 29.43, 29.47, 31.20, 31.68, 33.77, 33.88, 34.08, 36.24, 40.38, 42.49, 46.98, 47.71, 48.06, 50.06, 55.09, 62.58, 63.13, 63, 31, 63.48, 68.22, 69.24, 69.41, 69.51, 69.64, 69.67, 69.82, 69.97, 70.03, 70.26, 70.75, 71.05, 77.20, 78.96, 113.14, 128.46, 161.52, 162.07, 166.49, 169.52, 172.53, 172.91.

### Example 50

### Preparation of Compound 52

A mixture of compound 51 (300 mg) and TFA (0.6 ml) in DCM (2.4 ml) was stirred at room temperature for 3 hours. The reaction solution was washed with saturated aqueous NaHCO₃, dried, and concentrated in vacuo. The residue was purified by Prep HPLC to yield 160 mg of product: ¹³C NMR δ 14.13, 22.67, 24.85, 24.91, 29.14, 29.32, 29.50, 29.69, 31.88, 34.05, 34.23, 36.90, 36.89, 39.89, 39.95, 45.45, 47.51, 47.82, 55.27, 62.55, 63.51, 63.58, 64.10, 64.18, 66.08, 70.14, 70.23, 70.41, 70.49, 70.76, 71.27, 77.21, 113.34, 125.87, 129.28, 161.98, 167.34, 169.94, 172.74, 173.11.

### Example 51

### Preparation of Nucleic Acids-Nanoparticle Compositions

In this example, nanoparticle compositions carrying oligonucleotides including LNA were prepared. For example, cationic lipid, DOPE: Chol: compound 10 were mixed at molar ratio 18:60:20:2 in 10 mL of 90% ethanol (total lipid 30 μmole). Oligonucleotides (anti-BCl siRNA: SEQ ID NO: 2 and 3, 0.4 μmole) were dissolved in equal volume of 20 mM Tris buffer (pH 7.4-7.6). After being heated to 37° C., the two solutions were mixed together through a duel syringe pump and the mixed solution was subsequently diluted with 20 mL of 20 mM Tris buffer (300 mM NaCl, pH 7.4-7.6). The mixture was incubated at 37° C. for 30 minutes and dialyzed in 10 mM PBS buffer (138 mM NaCl, 2.7 mM KCl, pH 7.4). Stable particles were obtained after the removal of ethanol from the mixture by dialysis. The nanoparticle solution was concentrated by centrifugation. The nanoparticle solution was transferred into a 15 mL centrifugal filter device (Amicon Ultra-15, Millipore, USA). The centrifuge speed was at 3,000 rpm at 4° C. The concentrated suspension was collected and sterilized by filtration through a 0.22 μm syringe filter (Millex-GV, Millipore, USA). A homogeneous nanoparticle suspension was obtained.

The diameter and polydispersity of nanoparticle were measured at 25° in water (Sigma) as medium on a Plus 90 Particle Size Analyzer Dynamic Light Scattering Instrument (Brookhaven, N.Y.).

Nucleic acids encapsulation efficiency was determined by UV-VIS (Agilent 8453). The background UV-vis spectrum was obtained by scanning solution, which was a mixed solution composed of PBS buffer saline (250 μL), methanol (625 μL) and chloroform (250 μL). In order to determine the encapsulated nucleic acids concentration, methanol (625 μL) and chloroform (250 μL) were added to PBS buffer saline nanoparticle suspension (250 μL). After mixing, a clear solution was obtained and the solution was sonicated for 2 minutes before measuring absorbance at 260 nm. The encapsulated nucleic acids concentration and loading efficiency was calculated according to the equation (1) and (2):

Cₑₙ (μg/ml)=A₂₆₀×OD₂₆₀ unit (μg/mL)×dilution factor (μL/μL) (1)

where the dilution factor is given by the assay volume (4) divided by the sample stock volume (μL).

Encapsulation efficiency (%)=[Cₑₙ/Cᵢₙᵢₜᵢₐₗ]×100 (2)

where Cₑₙ is the nucleic acid (i.e., LNA oligonucleotide) concentration encapsulated in nanoparticle suspension after purification, and Cᵢₙᵢₜᵢₐₗ is the initial nucleic acid (LNA oligonucleotide) concentration before the formation of the nanoparticle suspension.

The particle size and polydispersity of various nanoparticle compositions are summarized in Table 5.

TABLE 5

| | | | | Carrier: | | Particle | | Zeta |
|---|---|---|---|---|---|---|---|---|
| Sample | Nanoparticle | Molar | Charge | Drug | | Size | Poly- | Potential |
| No. | Composition | Ratio | Ratio | (mole) | Oligo | (nm) | dispersity | (mV) |
| NP1 | Cholesterol: | 20:18:60:2 | 2.5:1 | 10:1 | Oligo-4 | 102 | 0.160 | +18 |
| | Cationic Lipid 1: | | | | | | | |
| | DOPE: | | | | | | | |
| | Compound 10 | | | | | | | |
| NP2 | Cationic Lipid 1: | 37:61:2 | 5:1 | 20:1 | Oligo-4 | 130 | 0.139 | +22 |
| | DOPE: | | | | | | | |
| | Compound 10 | | | | | | | |
| NP3 | Chololesterol: | 20:18:60:2 | 5:1 | 20:1 | Oligo-4 | 104 | 0.146 | +22 |
| | Cationic Lipid 1: | | | | | | | |
| | DOPE: | | | | | | | |
| | Compound 10 | | | | | | | |

### Example 52

### Nanoparticle Stability In pH 7.4 and 37° C.

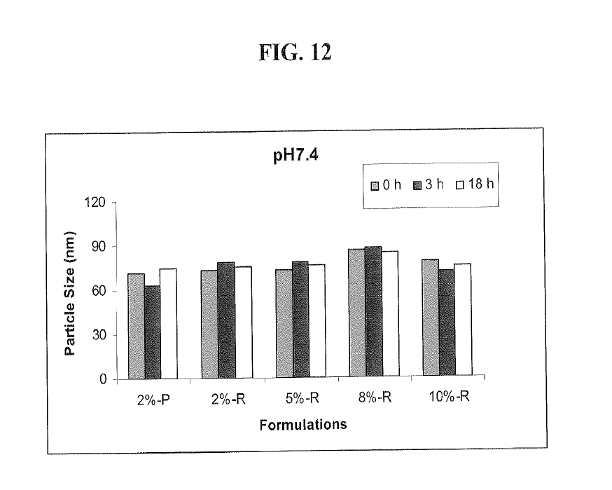

Stability of nanoparticles prepared according to the present invention was evaluated at pH7.4. Nanoparticle stability was defined as their capability to retain the structural integrity in PBS buffer at 37° C. over time. The colloidal stability of nanoparticles was evaluated by monitoring changes in the mean diameter over time. Nanoparticles containing 2-10% releasable polymeric lipids (compound 10) were dispersed in 10 mM PBS buffer (138 mM NaCl, 2.7 mM KCl, pH 7.4) and stored at 37° C. At a given time point, about 20-50 μL of the suspension was taken and diluted with pure water up to 2 mL. The sizes of nanoparticles were measured by DLS at 25° C. The results show that the nanoparticles containing compound 10 in 2-10% are stable at pH 7.4 which is comparable to storage, formulation, and normal body fluid condition. The results are set forth in FIG. 12.

### Example 53

### Nanoparticle Stability in Acidic pH

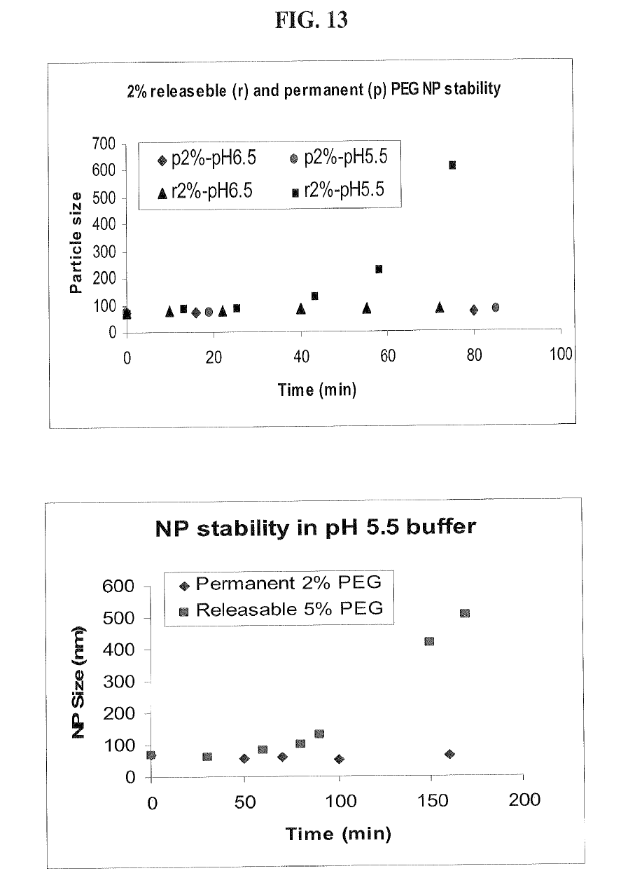

Stability of nanoparticles was evaluated in acidic environment. Changes in size of nanoparticles containing 2 or 5% releasable polymeric lipids (compound 10) or 2% permanently bonded polymeric lipids (compound 52) were measure in pH 6.5 and 5.5. The nanoparticles containing 2 or 5% releasable polymeric lipids (compound 10) were degraded significantly in acidic pH 5.5 as compared to nanoparticles containing permanently bonded polymeric lipids (compound 52). The nanoparticles containing permanently bonded polymeric lipids were very stable in pH 5.5. The results were set forth in FIG. 13. The results show that nanoparticles containing releasable polymeric lipids of the present invention enhance release of encapsulated active drugs in acidic environment such as in tumors and endosome. The nanoparticle can disrupt/rupture endosome and promote release of encapsulated nucleic acids into the cytoplasm. The nucleic acids encapsulated within the nanoparticles containing the permanently bonded polymeric lipids were trapped and were not available as compared to nanoparticles prepared according to the present invention. The results show that the nanoparticles prepared according to the present invention provides a means for increasing bioavailability of therapeutic agents at the target area.

### Example 54

### Nanoparticle Stability in Mouse Plasma

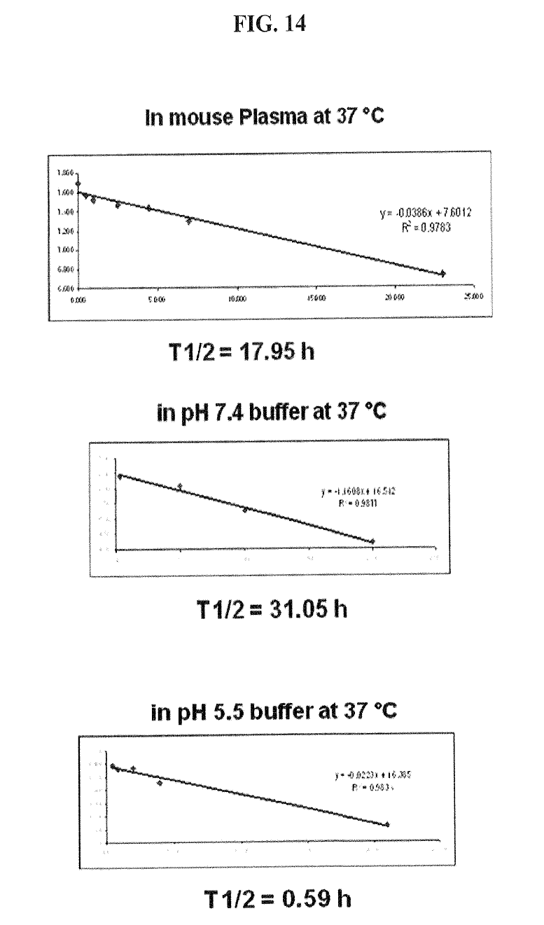

Stability of nanoparticles containing releasable polymeric lipids (compound 10) evaluated in mouse plasma. The results show that the half life of the nanoparticles in mouse plasma at 37° C. was about 17.95 hours. The half life of the nanoparticles in pH 7.4 and 5.5 buffer was 31.05 and 0.59 hour, respectively. The results are shown in FIG. 14. The results show that the nanoparticles according to the present invention are stable in physiological condition sufficient to circulate in the body and deliver nucleic acids to the target area. The very short half life in pH 5.5 buffer show that the nanoparticles stable in the physiological pH degrade rapidly in acidic environment such as cancer cells and endosome to facilitate release of encapsulated nucleic acids in the target area.

### Example 55

### Effects on Cellular Uptake and Cytoplasmic Localization of Nucleic Acids

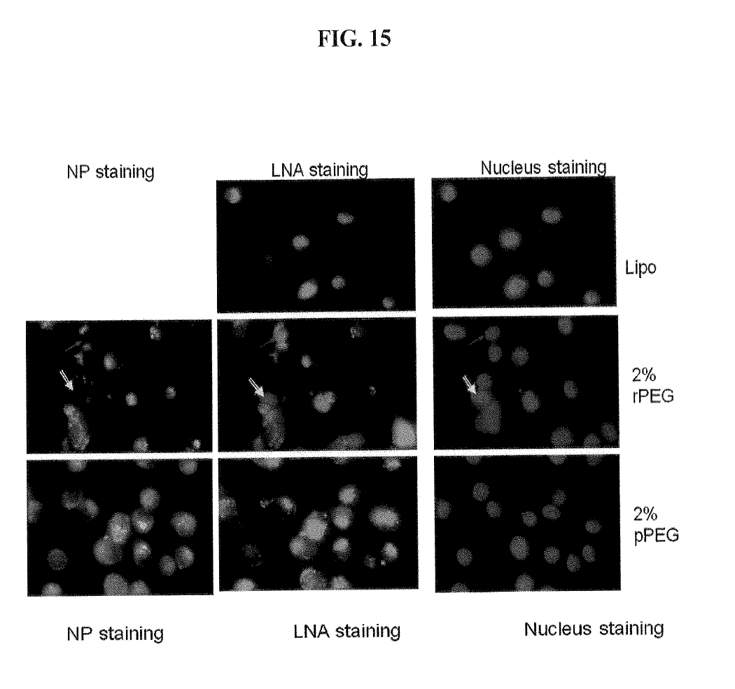

Effects of compounds described herein on cellular uptake and cytoplasmic localization of nucleic acids were evaluated in cells. Cancer cells were treated with nanoparticles containing 2% releasable polymeric lipids (compound 10) or 2% permanently bonded polymeric lipids (compound 52). The cells were washed, stained, and fixed. The samples were inspected under fluorescent microscope. Fluorescent images of the treated cell samples are shown in FIG. 15. In the images, oligonucleotides are shown in the cytosol and nucleus of the cells treated with the nanoparticles containing releasable polymeric lipids. The oligonucleotides were released from endosomes and diffused into the cytoplasm. The images show that the nanoparticles containing permanently bonded polymeric lipids did not show evidence of delivering nucleic acids to the nucleus. The results show that the nanoparticles containing releasable polymeric lipids are an effective means for delivering therapeutic nucleic acids into cells and localizing them in cellular compartments, cytoplasmic area and nucleus within cells.

### Example 56

### Effects of Increase in Amounts of Releasable Polymeric Lipids on Modulation of Target Gene Expression In Vitro

Effects of the amounts of the releasable polymeric lipids on modulation of target gene were evaluated using human prostate cancer cells (15PC3). Nanoparticle compositions with various amounts of releasable polymeric lipids (compound 10) are summarized in Table 6. Antisense ErbB3 oligonucleotides (SEQ ID NO: 6) were encapsulated within the nanoparticles.

TABLE 6

| Sample No. | NP4 | NP5 | NP6 | NP7 |
|---|---|---|---|---|
| Formulations | 2% r-PEG | 5% r-PEG | 8% r-PEG | 10% r-PEG |
| ζ potential (mV) | 19.42 | 14.74 | 9.75 | 10.55 |

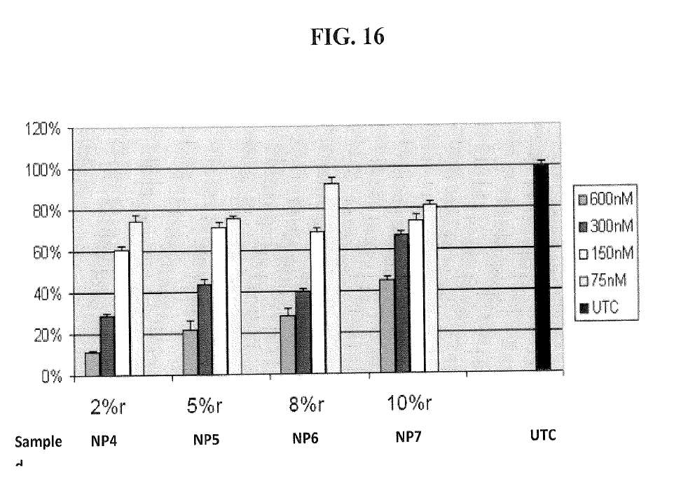

The results showed that nanoparticles including up to 10% releasable polymeric lipids inhibited expression of ErbB3 mRNA. The results are set forth in FIG. 16. Nanoparticles containing permanently bonded polymeric lipids lost efficacy on modulation of target gene expression when the amount of permanently bonded polymeric lipids was increased from 2% to 5%. (The data now shown). The encapsulated nucleic acids were not released from the nanoparticles containing permanently bonded polymeric lipids when the nanoparticles contained high amounts of polymeric lipids. The results show that the present invention allows nanoparticles to include high amount of polymeric lipids, if desired, compared to nanoparticles including permanently bonded polymeric lipids. It is advantageous because polymeric lipids extend circulation of the transport systems and decrease premature excretion from the body.

### Example 57

### In Vitro BCL2 mRNA Downregulation in Human Prostate Cancer Cells

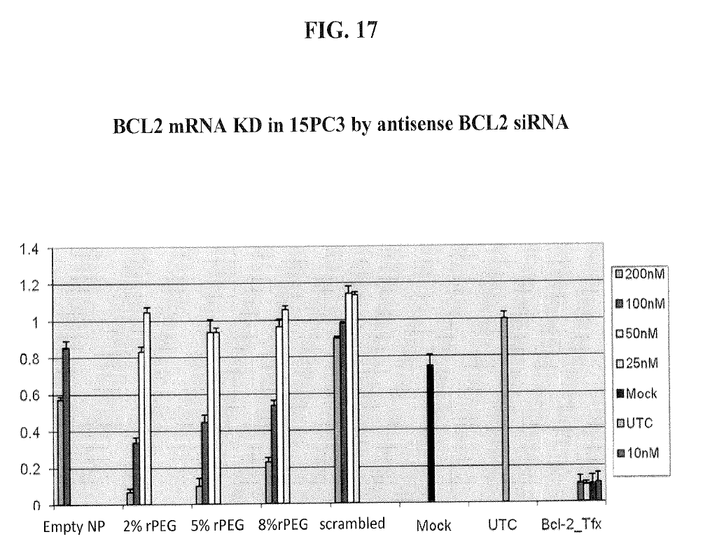

Effects of the compounds described herein on modulating target gene expression are evaluated in human prostate cancer cells (15PC3). Cells were treated with nanoparticles prepared by using NP1, NP2 and NP3 compositions, as described in Table 5 of Example 51. The nanoparticles contained antisense BCL2 siRNA oligomers (SEQ ID NOs: 2 and 3). Cells were also treated with nanoparticles with scrambled oligonucleotides, empty nanoparticles without oligonucleotides, or naked siRNA. The results showed that the antisense siRNA oligomer encapsulated within the nanoparticles containing 2, 5 and 8% releasable polymeric lipids inhibited BCL2 gene expression. The inhibition was concentration-dependent. The results are set forth in FIG. 17.

### Example 58

### In Vitro BCL2 mRNA Downregulation in Human Lung Cancer Cells

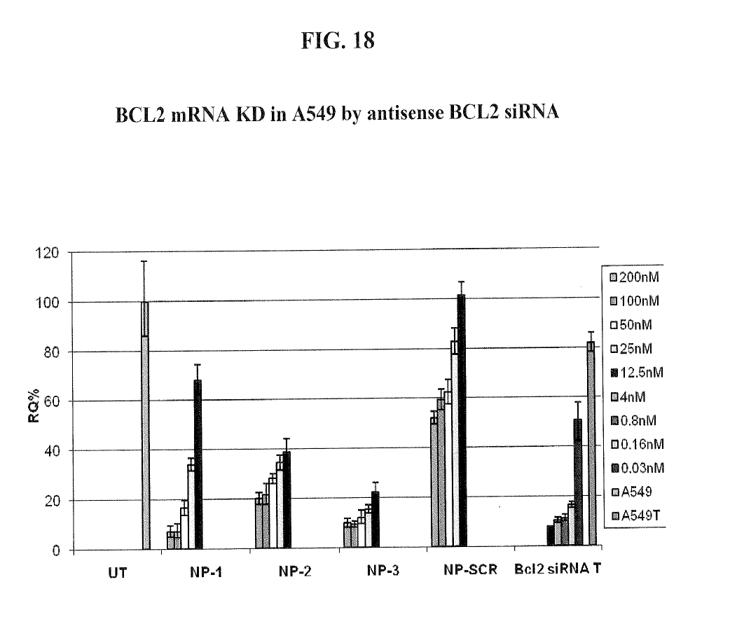

Effects of the compounds described herein on modulating target gene expression are evaluated in human lung cancer cells (A549). Cells were treated with nanoparticles containing antisense BCL2 siRNA oligomers (SEQ ID NOs: 2 and 3). The nanoparticles contained 2, 5 or 8% releasable polymeric lipids (compound 10). Cells were also treated with nanoparticles with scrambled oligonucleotides, or naked siRNA. The results showed that the antisense BCL2 siRNA oligomer encapsulated within the nanoparticles containing releasable polymeric lipids inhibited BCL2 gene expression. The inhibition was target sequence specific and dose-dependent. The results are set forth in FIG. 18.

### Example 59

### In Vitro ErbB3 mRNA Downregulation in Human Prostate Cancer Cells

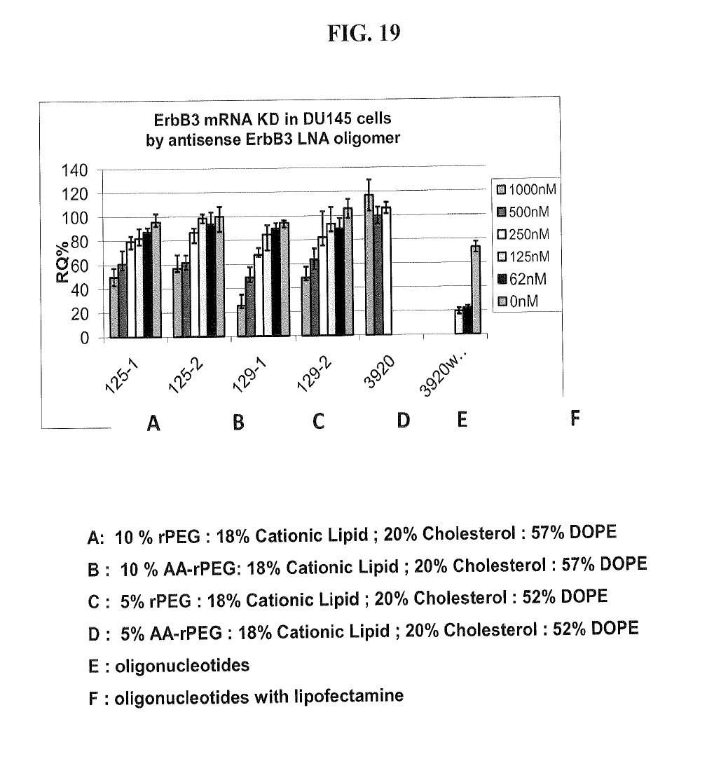

Effects of the compounds described herein on modulating target gene expression are evaluated in human prostate cancer cells (DU149). The cells were treated with nanoparticles containing antisense ErbB2 oligomers (SEQ ID NO: 6). The antisense oligomers include modified nucleic acids such as LNA and phosphorodiester linkages. The nanoparticles contained releasable polymeric lipids modified with a targeting group, animaside (compound 38). The cells were treated with the nanoparticles including 5 or 10% releasable polymeric lipids with animaside (compound 38) or without animaside (compound 10): a mixture of 18% cationic lipid 1: 20% cholesterol: 57% DOPE: 5% compound 10 or 38, or a mixture of 18% cationic lipid 1: 20% cholesterol: 52% DOPE: 10% compound 10 or 38. The results showed that the antisense ErbB3 oligomers encapsulated within the nanoparticles containing releasable polymeric lipids inhibited target gene expression. The inhibition was target sequence specific and dose-dependent. The results are set forth in FIG. 19.

### Example 60

### Effects on Modulation of Target Gene Expression In Vitro

Effects of the nanoparticles described herein on modulating target gene expression are evaluated in a number of different cancer cells including epidermoid carcinoma (A431), prostate cancer (15PC3, LNCaP, PC3, CWR22), lung cancer (A549, HCC827, H1581), breast cancer (SKBR3), colon cancer (SW480), pancreatic cancer cells (BxPC3), gastric cancer cells (N87), and melanoma (518A2). Cells are treated with nanoparticles containing compound 10 (with Oligo 2 or a scrambled sequence, Oligo-3). After treatment, the intracellular mRNA levels of the target gene, such as human ErbB3, and a housekeeping gene, such as GAPDH are quantitated by RT-qPCR. The expression levels of mRNA normalized to that of GAPDH are compared. To confirm the mRNA down-regulation data, the protein level from the cells are also analyzed using conjugates of both Oligo-2 and Oligo-3 by Western Blot method.

### Example 61

### Effects on Target Gene Downregulation In Vivo

Effects of the nanoparticles described herein on downregulating target gene expression are evaluated in mice xenografted with human cancer cells. Xenograft tumors are established in mice by injecting human cancer cells. 15PC3 human prostate tumors are established in nude mice by subcutaneous injection of 5×10⁶ cells/mouse into the right auxiliary flank. When tumors reach approximately 100 mm³, the mice are treated with nanoparticles containing compound 10 or 38 (with Oligo 2) intravenously (i.v.) (alternatively, intraperitoneally) or at 60 mg/kg, 45 mg/kg, 30 mg/kg, 25 mg/kg, 15 mg/kg, or 5 mg/kg/dose (equivalent of Oligo2) at q3 d×4 or more. The dosage is based on the amounts of oligonucleotides contained in the nanoparticles. The mice are sacrificed twenty four hours after the final dose. Plasma samples are collected from the mice and stored at −20° C. Tumor and liver samples are also collected from the mice. The samples were analyzed for mRNA KD.

1. A compound of Formula (I):
R-(L₁)ₐ-M-(L₂)_{b}-Q
wherein
R is a non-antigenic polymer;
L₁₋₂ are independently selected bifunctional linkers;
M is an acid labile linker;
Q is a substituted or unsubstituted saturated or unsaturated C4-30-containing moiety;
(a) is zero or a positive integer; and
(b) is zero or a positive integer,
wherein a targeting group is optionally linked to the non-antigenic polymer.

2. The compound of claim 1, wherein M is a ketal- or acetal containing moiety or an imine-containing moiety.

3. The compound of claim 1, wherein M is —CR₃R₄—O—CR₁R₂—O—CR₅R₆—,
wherein
R₁₋₂ are independently selected from the group consisting of hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, and substituted arylcarbonyloxy; and
R₃₋₆ are independently selected from the group consisting of hydrogen, amine, substituted amine, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, and substituted arylcarbonyloxy.

4. (canceled)

5. The compound of claim 1, wherein M is —N═CR₁₀— or —CR₁₀═N—, wherein R₁₀ is hydrogen, C₁₋₆ alkyl, C₃₋₈ branched alkyl, C₃₋₈ cycloalkyl, C₁₋₆ substituted alkyl, C₃₋₈ substituted cycloalkyl, aryl and substituted aryl.

6. The compound of claim 1, wherein R is a polyalkylene oxide.

7. (canceled)

8. The compound of claim 1, wherein Q has the structure of Formula (Ia):
(Ia) 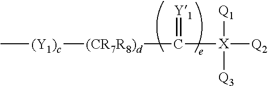
wherein
Y₁ and Y′₁ are independently O, S or NR₃₁;
(c) is 0 or 1;
(d) is 0 or a positive integer;
(e) is 0 or 1;
X is C, N or P;
Q₁ is H, C₁₋₃ alkyl, NR₃₂, OH, or 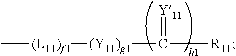
Q₂ is H, C₁₋₃ alkyl, NR₃₃, OH, or 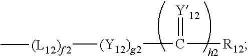
Q₃ is a lone electron pair, (═O), H, C₁₋₃ alkyl, NR₃₄, OH, or 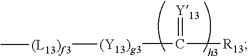
provided that
(i) when X is C, Q₃ is not a lone electron pair or (═O);
(ii) when X is N, Q₃ is a lone electron pair; and
(iii) when X is P, Q₃ is Q₃ is (═O) and (e) is 0,
wherein
L₁₁, L₁₂ and L₁₃ are independently selected bifunctional spacers;
Y₁₁, Y′₁₁, Y₁₂, Y′₁₂, Y₁₃, and Y′₁₃ are independently O, S or NR₃₅;
R₁₁, R₁₂ and R₁₃ are independently saturated or unsaturated C₄₋₃₀;
(f1), (f2) and (f3) are independently 0 or 1;
(g1), (g2) and (g3) are independently 0 or 1; and
(h1), (h2) and (h3) are independently or 1;
R₇₋₈ are independently selected hydrogen, hydroxyl, amine, substituted amine, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, and substituted C₁₋₆ heteroalkyl; and
R₃₁₋₃₅ are independently selected hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, and substituted C₁₋₆ heteroalkyl,
provided that Q includes at least one or two of R₁₁, R₁₂ and R₁₃.

9. The compound of claim 8, having Formula (II):

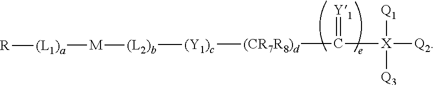

10. The compound of claim 8, having Formula (IIa):

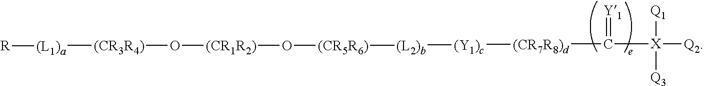

11. The compound of claim 8, having Formula (IIb) or (II′b):

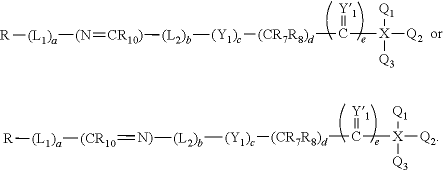

12. The compound of claim 8, wherein Q₁₋₃ independently include groups selected from C12-22 alkyl, C12-22 alkenyl, C12-22 alkyloxy, auroyl (C12), myristoyl (C14), palmitoyl (C16), stearoyl (C18), oleoyl (C18), and erucoyl (C22); saturated or unsaturated C12 alkyloxy, C14 alkyloxy, C16 alkyloxy, C18 alkyloxy, C20 alkyloxy, and C22 alkyloxy; and saturated or unsaturated C12 alkyl, C14 alkyl, C16 alkyl, C18 alkyl, C20 alkyl and C22 alkyl.

13. The compound of claim 8, wherein L₁₁, L₁₂ and L₁₃ are independently selected from the group consisting of:
—(CR₃₁R₃₂)_{q1},
—Y₂₆(CR₃₁R₃₂)_{q1}—
—CH₂—, —(CH₂)₂—, —(CH₂)₃—, —(CH₂)₄—, —(CH₂)₅—, —(CH₂)₆—, —O(CH₂)₃—, —O(CH₂)₄—, —O(CH₂)₅—, —O(CH₂)₆—, and CH(OH)—,
wherein:
Y₂₆ is O, NR₃₃, or S;
R₃₁₋₃₂ are independently selected from the group consisting of hydrogen, hydroxyl, C₁₋₆ alkyls, C₃₋₁₂ branched alkyls, C₃₋₈ cycloalkyls, C₁₋₆ substituted alkyls, C₃₋₈ substituted cycloalkyls, C₁₋₆ heteroalkyls, substituted C₁₋₆ heteroalkyls, C₁₋₆alkoxy, phenoxy and C₁₋₆ heteroalkoxy;
R₃₃ is selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, C₁₋₆ heteroalkyls, substituted C₁₋₆ heteroalkyls, C₁₋₆ alkoxy, phenoxy and C₁₋₆ heteroalkoxy; and
(q1) is zero or a positive integer.

14. (canceled)

15. The compound of claim 1, wherein L₁ is selected from the group consisting of:
—(CR₂₁R₂₂)ₜ₁-[C(═Y₁₆)]ₐ₃—,
—(CR₂₁R₂₂)ₜ₁Y₁₇—(CR₂₃R₂₄)ₜ₂—(Y₁₈)ₐ₂-[C(═Y₁₆)]ₐ₃—,
—(CR₂₁R₂₂CR₂₃R₂₄Y₁₇)ₜ₁[C(═Y₁₆)]ₐ₃—,
—(CR₂₁R₂₂CR₂₃R₂₄Y₁₇)ₜ₁(CR₂₅R₂₆)ₜ₄—(Y₁₈)ₐ₂-[C(═Y₁₆)]ₐ₃—,
—[(CR₂₁R₂₂CR₂₃R₂₄)ₜ₂Y₁₇]ₜ₃(CR₂₅R₂₆)ₜ₄—(Y₁₈)ₐ₂-[C(═Y₁₆)]ₐ₃—,
—(CR₂₁R₂₂)ₜ₁-[(CR₂₃R₂₄)ₜ₂Y₁₇]ₜ₃(CR₂₅R₂₆)ₜ₄—(Y₁₈)ₐ₂-[C(═Y₁₆)]ₐ₃—,
—(CR₂₁R₂₂)ₜ₁(Y₁₇)ₐ₂[C(═Y₁₆)]ₐ₃(CR₂₃R₂₄)ₜ₂—,
—(CR₂₁R₂₂)ₜ₁(Y₁₇)ₐ₂[C(═Y₁₆)]ₐ₃Y₁₄(CR₂₃R₂₄)ₜ₂—,
—(CR₂₁R₂₂)ₜ₁(Y₁₇)ₐ₂[C(═Y₁₆)]ₐ₃(CR₂₃R₂₄)ₜ₂—Y₁₅—(CR₂₃R₂₄)ₜ₃—,
—(CR₂₁R₂₂)ₜ₁(Y₁₇)ₐ₂[C(═Y₁₆)]ₐ₃Y₁₄ (CR₂₃R₂₄)ₜ₂—Y₁₅—(CR₂₃R₂₄)ₜ₃—,
—(CR₂₁R₂₂)ₜ₁(Y₁₇)ₐ₂[C(═Y₁₆)]ₐ₃(CR₂₃R₂₄CR₂₅R₂₆Y₁₉)ₜ₂(CR₂₇CR₂₈)ₜ₃—,
—(CR₂₁R₂₂)ₜ₁(Y₁₇)ₐ₂[C(═Y₁₆)]ₐ₃Y₁₄(CR₂₃R₂₄CR₂₅R₂₆Y₁₉)ₜ₂(CR₂₇CR₂₈)ₜ₃—, 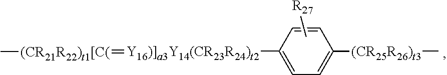
—CH₂—, —(CH₂)₂—, —(CH₂)₃—, —(CH₂)₄—, —(CH₂)₅—, —(CH₂)₆—, —NH(CH₂)—
—CH(NH₂)CH₂—,
—(CH₂)₄—C(═O)—, —(CH₂)₅—C(═O)—, —(CH₂)₆—C(═O)—, —NH(CH₂)—
—CH₂CH₂O—CH₂O—C(═O)—, —(CH₂CH₂O)₂—CH₂O—C(═O)—,
—(CH₂CH₂O)₃—CH₂O—C(═O)—,
—(CH₂CH₂O)₂—C(═O)—,
—CH₂CH₂O—CH₂CH₂NH—C(═O)—,
—(CH₂CH₂O)₂—CH₂CH₂NH—C(═O)—,
—CH₂—O—CH₂CH₂O—CH₂CH₂NH—C(═O)—,
—CH₂—O—(CH₂CH₂O)₂—CH₂CH₂NH—C(═O)—,
—CH₂—O—CH₂CH₂O—CH₂C(═O)—,
—CH₂—O—(CH₂CH₂O)₂—CH₂C(═O)—,
—(CH₂)₄—C(═O)NH—, —(CH₂)₅—C(═O)NH—,
—(CH₂)₆—C(═O)NH—,
—CH₂CH₂O—CH₂O—C(═O)—NH—,
—(CH₂CH₂O)₂—CH₂O—C(═O)—NH—,
—(CH₂CH₂O)₃—CH₂O—C(═O)—NH—,
—(CH₂CH₂O)₂—C(═O)—NH—,
—CH₂CH₂O—CH₂CH₂NH—C(═O)—NH—,
—(CH₂CH₂O)₂—CH₂CH₂NH—C(═O)—NH—,
—CH₂—O—CH₂CH₂O—CH₂CH₂NH—C═O—NH—,
—CH₂—O—(CH₂CH₂O)₂—CH₂CH₂NH—C(═O)—NH—,
—CH₂—O—CH₂CH₂O—CH₂C(═O)—NH—,
—CH₂—O—(CH₂CH₂O)₂—CH₂C(═O)—NH—,
—(CH₂CH₂O)₂—, —CH₂CH₂O—CH₂O—,
—(CH₂CH₂O)₂—CH₂CH₂NH—,
—(CH₂CH₂O)₃—CH₂CH₂NH —,
—CH₂CH₂O—CH₂CH₂NH—,
—(CH₂CH₂O)₂—CH₂CH₂NH—,
—CH₂—O—CH₂CH₂O—CH₂CH₂NH—,
—CH₂—O—(CH₂CH₂O)₂—CH₂CH₂NH—,
—CH₂—O—CH₂CH₂O—,
—CH₂—O—(CH₂CH₂O)₂—, 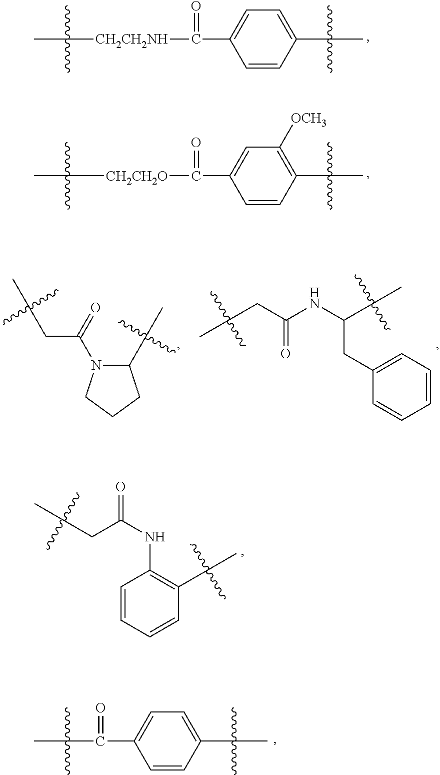
—C(═O)NH(CH₂)₂—, —CH₂C(═O)NH(CH₂)₂—,
—C(═O)NH(CH₂)₃—, —CH₂C(═O)NH(CH₂)₃—,
—C(═O)NH(CH₂)₄—, —CH₂C(═O)NH(CH₂)₄—,
—C(═O)NH(CH₂)₅—, —CH₂C(═O)NH(CH₂)₅—,
—C(═O)NH(CH₂)₆—, —CH₂C(═O)NH(CH₂)₆—,
—C(═O)O(CH₂)₂—, —CH₂C(═O)O(CH₂)₂—,
—C(═O)O(CH₂)₃—, —CH₂C(═O)O(CH₂)₃—,
—C(═O)O(CH₂)₄—, —CH₂C(═O)O(CH₂)₄—,
—C(═O)O(CH₂)₅—, —CH₂C(═O)O(CH₂)₅—,
—C(═O)O(O₂)₆—, —CH₂C(═O)O(CH₂)₆—,
—(CH₂CH₂)₂NHC(═O)NH(CH₂)₂—,
—(CH₂CH₂)₂NHC(═O)NH(CH₂)₃—,
—(CH₂CH₂)₂NHC(═)NH(CH₂)₄—,
—(CH₂CH₂)₂NHC(═O)NH(CH₂)₅—,
—(CH₂CH₂)₂NHC(═O)NH(CH₂)₆—,
—(CH₂CH₂)₂NHC(═O)O(CH₂)₂—,
—(CH₂CH₂)₂NHC(═O)O(CH₂)₃—,
—(CH₂CH₂)₂NHC(═O)O(CH₂)₄—,
—(CH₂CH₂)₂NHC(═)O(CH₂)₅—,
—(CH₂CH₂)₂NHC(═O)O(CH₂)₆—,
—(CH₂CH₂)₂NHC(═O)(CH₂)₂—,
—(CH₂CH₂)₂NHC(═O)(CH₂)₃—,
—(CH₂CH₂)₂NHC(═O)(CH₂)₄—,
—(CH₂CH₂)₂NHC(═O)(CH₂)₅—, and
—(CH₂CH₂)₂NHC(═O)(CH₂)₆—
wherein:
Y₁₆ is O, NR₂₈, or S;
Y₁₄₋₁₅ and Y₁₇₋₁₉ are independently O, NR₂₉, or S;
R₂₁₋₂₇ are independently selected from the group consisting of hydrogen, hydroxyl, amine, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy; and
R₂₈₋₂₉ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
(t1), (t2), (t3) and (t4) are independently zero or positive integers; and
(a2) and (a3) are independently zero or 1.

16. (canceled)

17. The compound of claim 1, wherein L₂ is selected from the group consisting of:
—(CR′₂₁R′₂₂)_{t′1}-[C(═Y′₁₆)]_{a′3}(CR′₂₇CR′₂₈)_{t′2}—,
—(CR′₂₁R′₂₂)_{t′1}Y′₁₄—(CR′₂₃R′₂₄)_{t′2}-(═Y′₁₅)_{a′2}-[C(═Y′₁₆)]_{a′3}(CR′₂₇CR′₂₈)_{t′3}—,
—(CR′₂₁R′₂₂CR′₂₃R′₂₄Y′₁₄)_{t′1}-[C(═Y′₁₆)]_{a′3}(CR′₂₇CR′₂₈)_{t′2}—,
—(CR′₂₁R′₂₂CR′₂₃R′₂₄Y′₁₄)_{t′1}(CR′₂₅R′₂₆)_{t′2}-(═Y′₁₅)_{a′2}-[C(═Y′₁₆)]_{a′3}(CR′₂₇CR′₂₈)_{t′3}—,
—[(CR′₂₁R′₂₂CR′₂₃R′₂₄)_{t′2}Y′₁₄]_{t′1}(CR′₂₅R′₂₆)_{t′2}—(Y′₁₅)_{a′2}-[C(═Y′₁₆)]_{a′3}(CR′₂₇CR′₂₈)_{t′3}—,
—(CR′₂₁R′₂₂)_{t′1}—[(CR′₂₃R′₂₄)_{t′2}Y′₁₄]_{t′2}(CR′₂₅R′₂₆)_{t′3}-(Y′₁₅)_{a′2}-[C(═Y′₁₆)]_{a′3}(CR′₂₇CR′₂₈)_{t′4}—,
—(CR′₂₁R′₂₂)_{t′1}(Y′₁₄)_{a′2}[C(═Y′₁₆)]_{a′3}(CR′₂₃R′₂₄)_{t′2}—,
—(CR′₂₁R′₂₂)_{t′1}(Y′₁₄)_{a′2}[C(═Y′₁₆)]_{a′3}Y′₁₅(CR′₂₃—R′₂₄)_{t′2}—,
—(CR′₂₁R′₂₂)_{t′1}(Y′₁₄)_{a′2}[C(═Y′₁₆)]_{a′3}(CR′₂₃R′₂₄)_{t′2}—Y′₁₅—(CR′₂₃R′₂₄)_{t′3}—,
—(CR′₂₁R′₂₂)_{t′1}(Y′₁₄)_{a′2}[C(═Y′₁₆)]_{a′3}Y′₁₄(CR′₂₃R′₂₄)_{t′2}—Y′₁₅—(CR′₂₃R′₂₄)_{t′3}—,
—(CR′₂₁R′₂₂)_{t′1}(Y′₁₄)_{a′2}[C(═Y′₁₆)]_{a′3}(CR′₂₃R′₂₄CR′₂₅R′₂₆Y′₁₅)_{t′2}(CR′₂₇CR′₂₈)_{t′3}—,
—(CR′₂₁R′₂₂)_{t′1}(Y′₁₄)_{a′2}[C(═Y′₁₆)]_{a′3}Y′₁₇(CR′₂₃R′₂₄CR′₂₅R′₂₆Y′₁₅)_{t′2}(CR′₂₇CR′₂₈)_{t′3}—, 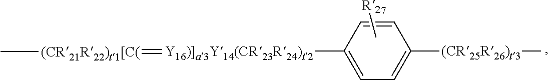
—CH₂—, —(CH₂)₂—, —(CH₂)₃—, —(CH₂)₄—, —(CH₂)₅—, —(CH₂)₆—, —NH(CH₂)—
—CH(NH₂)CH₂—,
—O(CH₂)₂—, —C(═O)O(CH₂)₃—, —C(═O)NH(CH₂)₃—,
—C(═O)(CH₂)₂—, —C(═O)(CH₂)₃—,
—CH₂—C(═O)—O(CH₂)₃—,
—CH₂—C(═O)—NH(CH₂)₃—,
CH₂—OC(═O)—O(CH₂)₃—,
—CH₂—OC(═O)—NH(CH₂)₃—,
—(CH₂)₂—C(═O)—O(CH₂)₃—,
—(CH₂)₂—C(═O)—NH(CH₂)₃—,
—CH₂C(═O)O(CH₂)₂—O—(CH₂)₂—,
—CH₂C(═O)NH(CH₂)₂—O—(CH₂)₂—,
—(CH₂)₂C(═O)O(CH₂)₂—-(CH₂)₂—,
—(CH₂)₂C(═O)NH(CH₂)₂—O—(CH₂)₂—,
—CH₂C(═O)O(CH₂CH₂O)₂CH₂CH₂—,
—(CH₂)₂C(═O)O(CH₂CH₂O)₂CH₂CH₂—,
—(CH₂CH₂O)₂—, —CH₂CH₂O—CH₂O—,
—(CH₂CH₂O)₂—CH₂CH₂NH—, —(CH₂CH₂O)₃—CH₂CH₂NH—,
—CH₂CH₂O—CH₂CH₂NH—,
—CH₂—O—CH₂CH₂O—CH₂CH₂NH—,
—CH₂—O—(CH₂CH₂O)₂—CH₂CH₂NH—,
—CH₂—O—CH₂CH₂O—, —CH₂—O—(CH₂CH₂O)₂—, 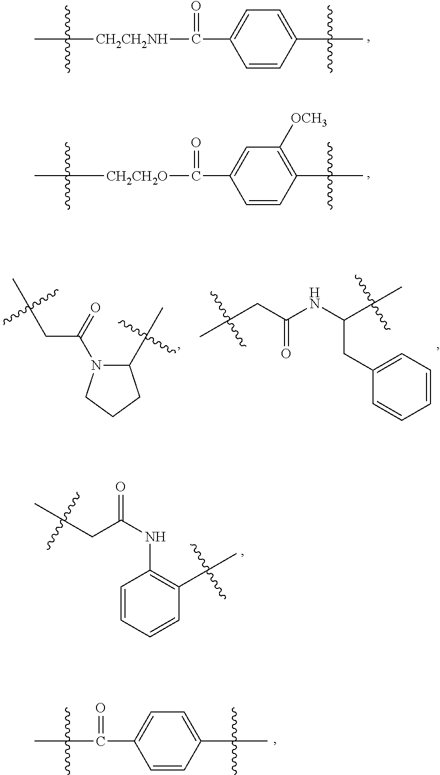
—(CH₂)₂NHC(═O)—(CH₂CH₂O)₂—,
—C(═O)NH(CH₂)₂—, —CH₂C(═O)NH(CH₂)₂—,
—C(═O)NH(CH₂)₃—, —CH₂C(═O)NH(CH₂)₃—,
—C(═O)NH(CH₂)₄—, —CH₂C(═O)NH(CH₂)₄—,
—C(═O)NH(CH₂)₅—, —CH₂C(═O)NH(CH₂)₅—,
—C(═O)NH(CH₂)₆—, —CH₂C(═O)NH(CH₂)₆—,
—C(═O)O(CH₂)₂—, —CH₂C(═O)O(CH₂)₂—,
—C(═O)O(CH₂)₂—, —CH₂C(═O)O(CH₂)₃—,
—C(═O)O(CH₂)₄—, —CH₂C(═O)O(CH₂)₄—,
—C(═O)O(CH₂)₅—, —CH₂C(═O)(CH₂)₅—,
—C(═O)O(CH₂)₆—, —CH₂C(═O)O(CH₂)₆—,
—(CH₂CH₂)₂NHC(═O)NH(CH₂)₂—,
—(CH₂CH₂)₂NHC(═O)NH(CH₂)₃—,
—(CH₂CH₂)₂NHC(═O)NH(CH₂)₄—,
—(CH₂CH₂)₂NHC(═O)NH(CH₂)₅—,
—(CH₂CH₂)₂NHC(═O)NH(CH₂)₆—,
—(CH₂CH₂)₂NHC(═O)O(CH₂)₂—,
—(CH₂CH₂)₂NHC(═O)O(CH₂)₃—,
—(CH₂CH₂)₂NHC(═O)O(CH₂)₄—,
—(CH₂CH₂)₂NHC(═O)O(CH₂)₅—,
—(CH₂CH₂)₂NHC(═O)O(CH₂)₆—,
—(CH₂CH₂)₂NHC(═O)(CH₂)₂—,
—(CH₂CH₂)₂NHC(═O)(CH₂)₃—,
—(CH₂CH₂)₂NHC(═O)(CH₂)₄—,
—(CH₂CH₂)₂NHC(═O)(CH₂)₅—, and
—(CH₂CH₂)₂NHC(═O)(CH₂)₆—,
wherein:
Y′₁₆ is O, NR′₂₈, or S;
Y′₁₄₋₁₅ and Y′₁₇ are independently O, NR′₂₉, or S;
R′₂₁₋₂₇ are independently selected from the group consisting of hydrogen, hydroxyl, amine, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆heteroalkoxy;
R′₂₈₋₂₉ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆heteroalkoxy;
(t′1), (t′2), (t′3) and (t′4) are independently zero or positive integers; and
(a′2) and (a′3) are independently zero or 1.

18. (canceled)

19. The compound of claim 8, wherein Q is selected from the group consisting of: 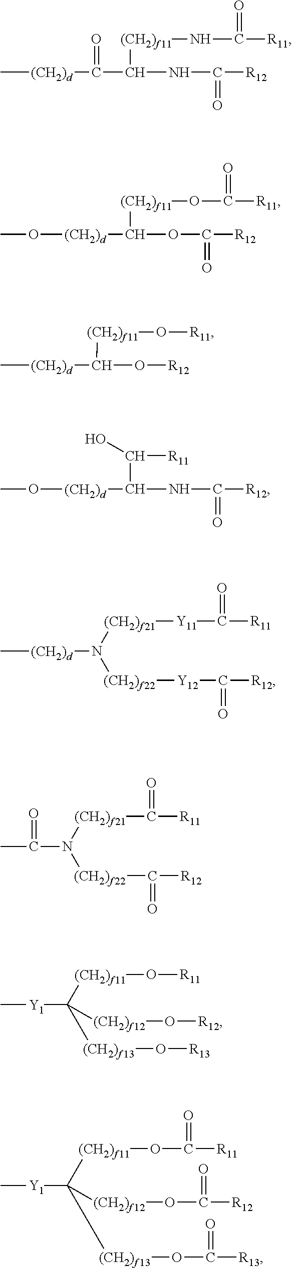 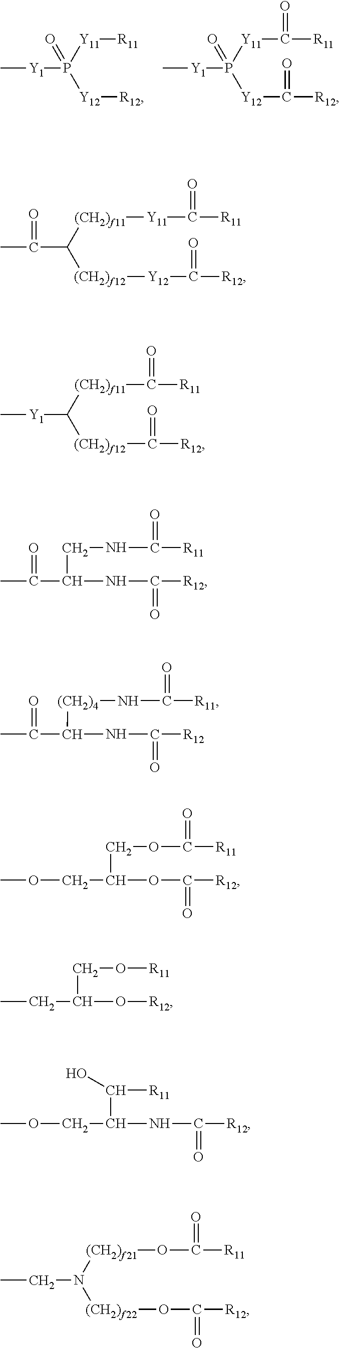 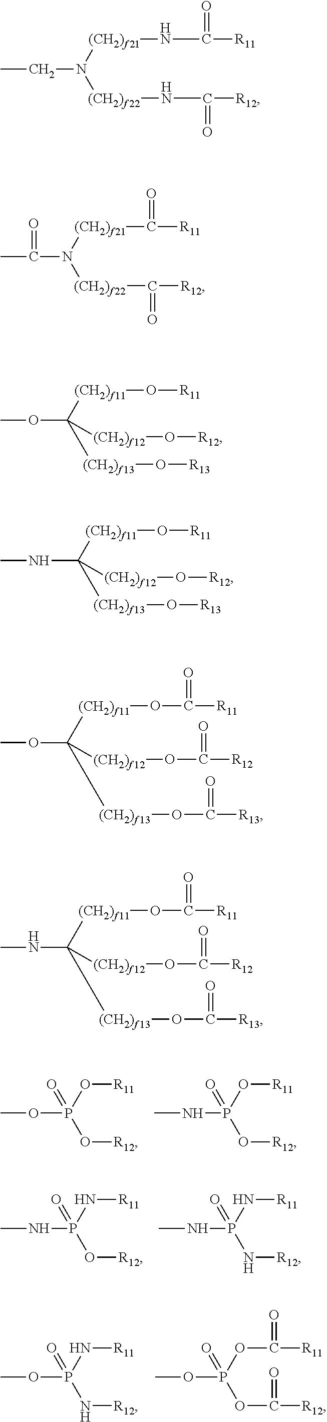 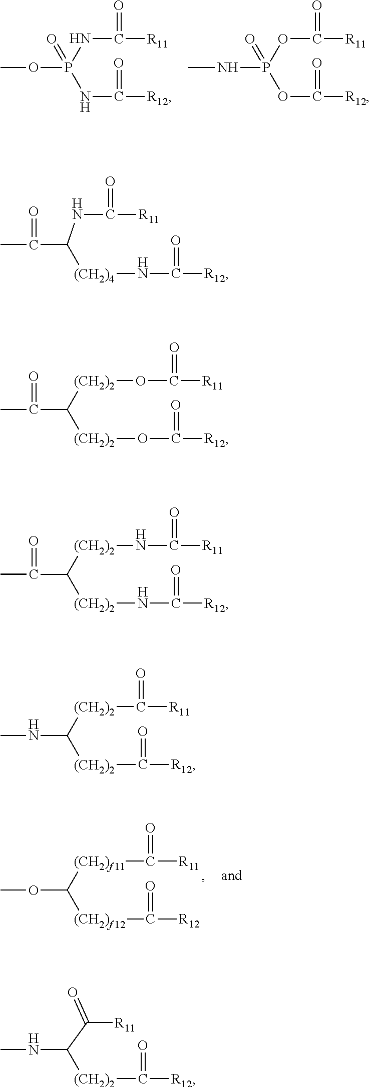
wherein
Y₁ is O, S, or NR₃₁;
R₁₁, R₁₂, and R₁₃ are independently substituted or unsubstituted, saturated or unsaturated C₄₋₃₀, the same or different C12-22 saturated or unsaturated aliphatic hydrocarbons;
R₃₁ is hydrogen, methyl or ethyl;
(d) is 0 or a positive integer; and
(f11), (f12) and (f13) are independently 0, 1, 2, 3, or 4; and
(f21) and (f22) are independently 1, 2, 3 or 4.

20. (canceled)

21. The compound of claim 1, wherein a targeting group is attached to the R group, and the compound of 1 having the formula:
A-R-(L₁)ₐ-M-(L₂)_{b}-Q,
wherein A is a targeting group.

22. (canceled)

23. The compound of claim 21, wherein the targeting group is selected from the group consisting of RGD peptides, folate, anisamide, vascular endothelial cell growth factor, FGF2, somatostatin and somatostatin analogs, transferrin, melanotropin, ApoE and ApoE peptides, von Willebrand's Factor and von Willebrand's Factor peptides, adenoviral fiber protein and adenoviral fiber protein peptides, PD1 and PD1 peptides, EGF and EGF peptides.

24. The compound of claim 8 having Formulas (IIIa), (IIIb), or (IIIb′): 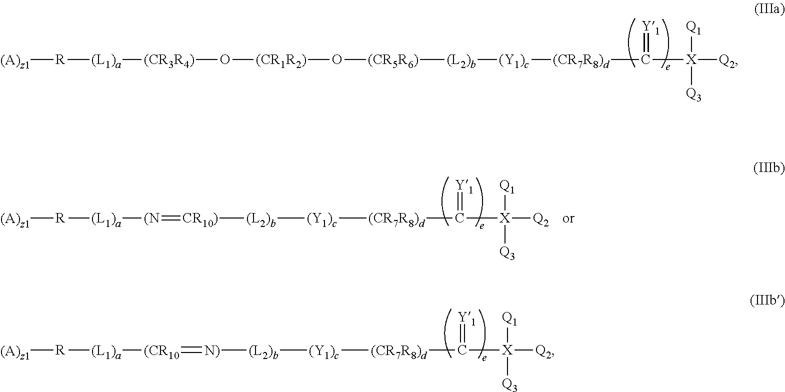
wherein A is a targeting group and (z1) is zero or 1.

25. (canceled)

26. The compound of claim 1 selected from the group consisting of: 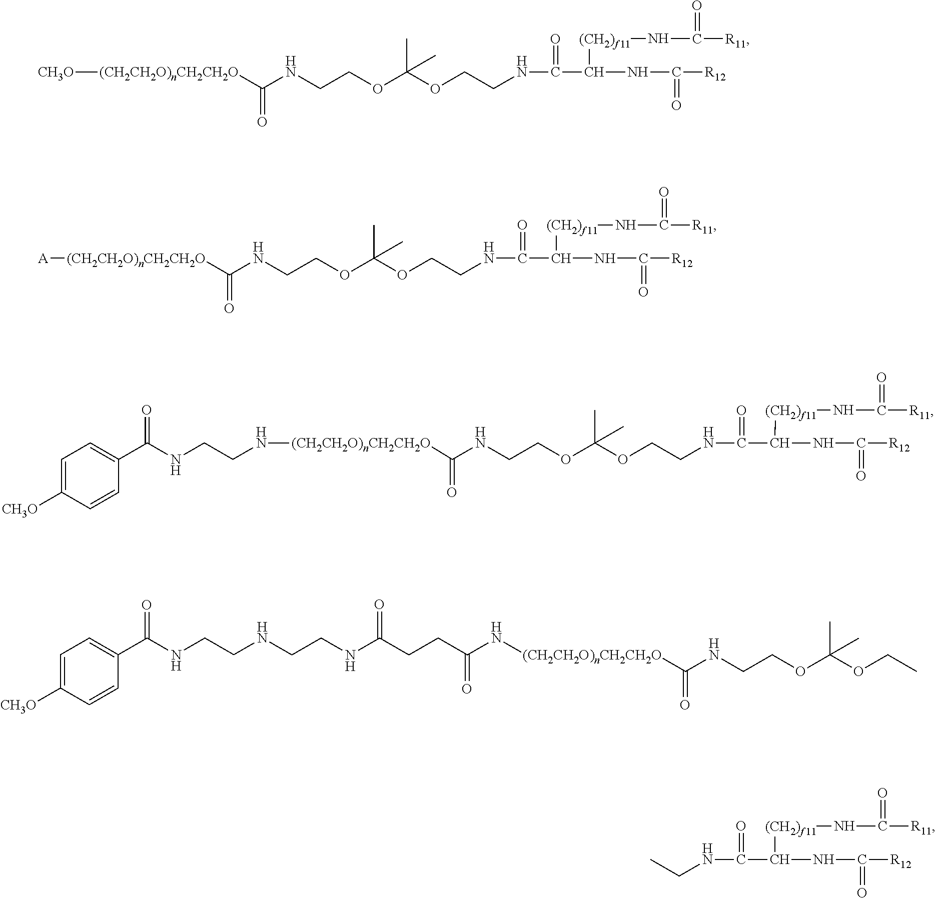 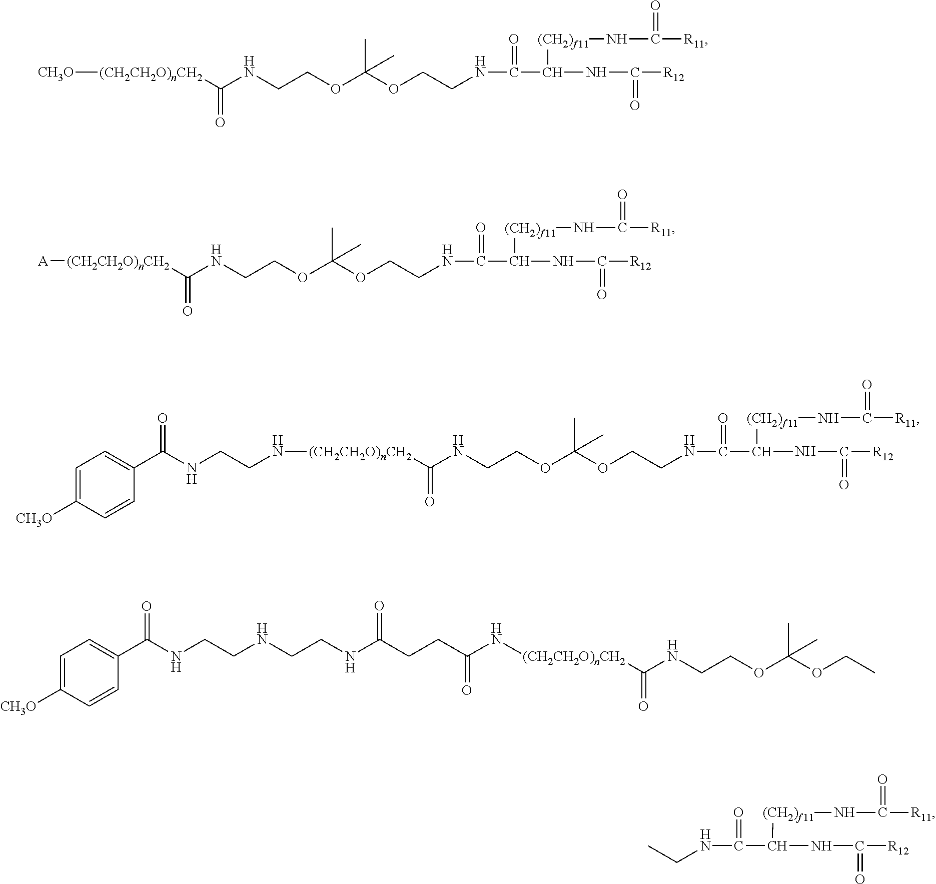 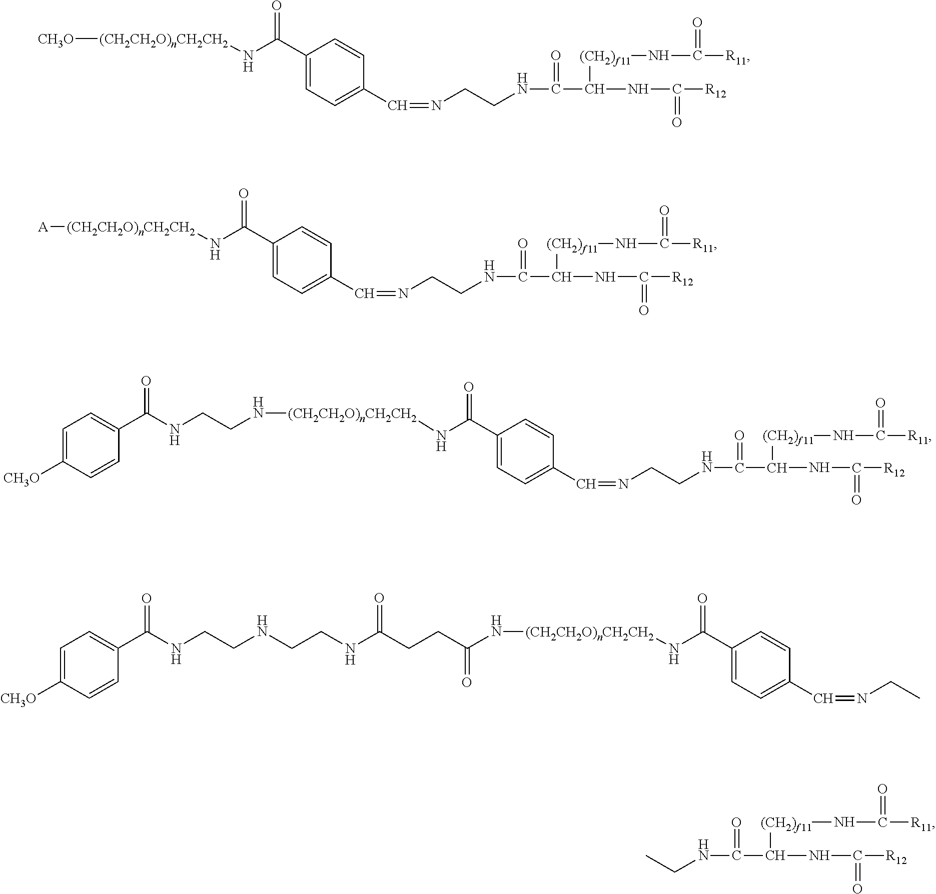 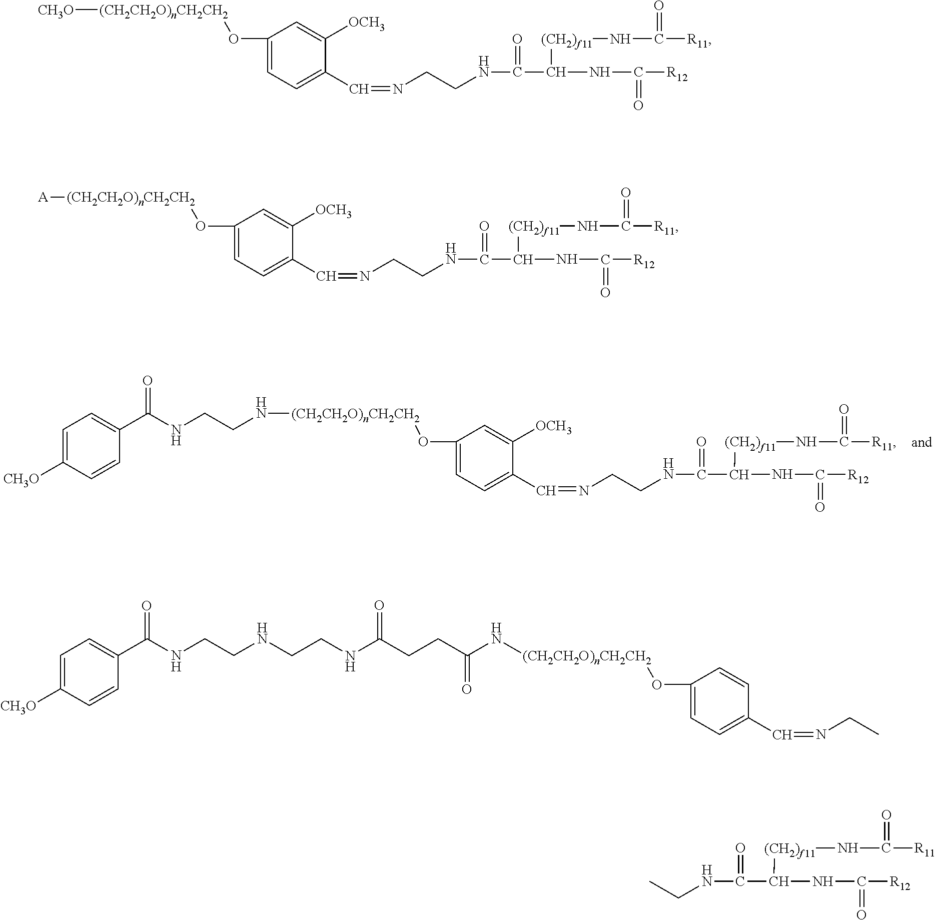
wherein
A is a targeting group;
(x) is the degree of polymerization so that the polymeric portion has the average molecular weight of from about 500 to about 5,000;
(f11) is zero, 1, 2, 3, or 4; and
R₁₁ and R₁₂ are independently C8-22 alkyl, C8-22 alkenyl, or C8-22 alkoxy.

27. The compound of claim 1 selected from the group consisting of: 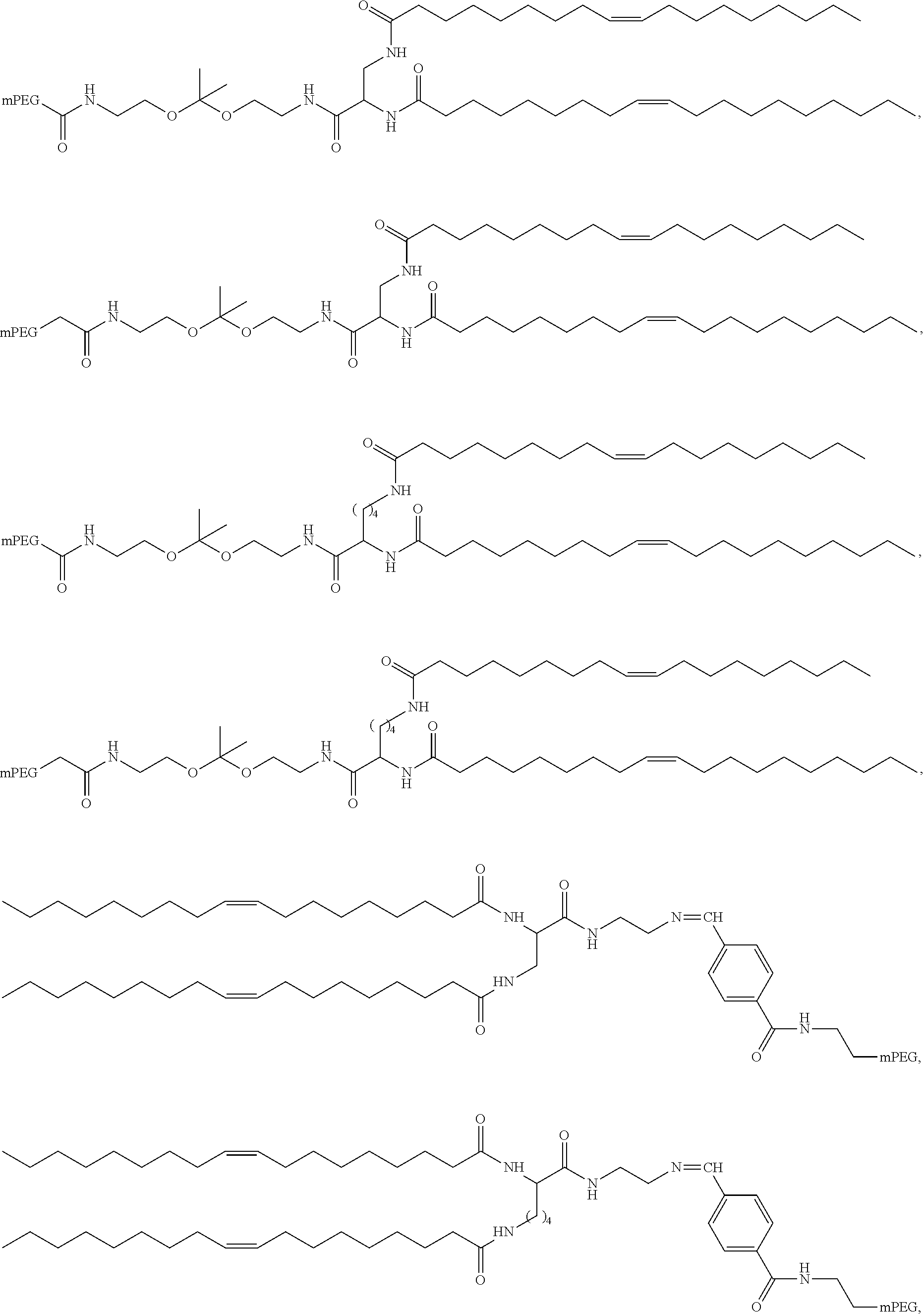 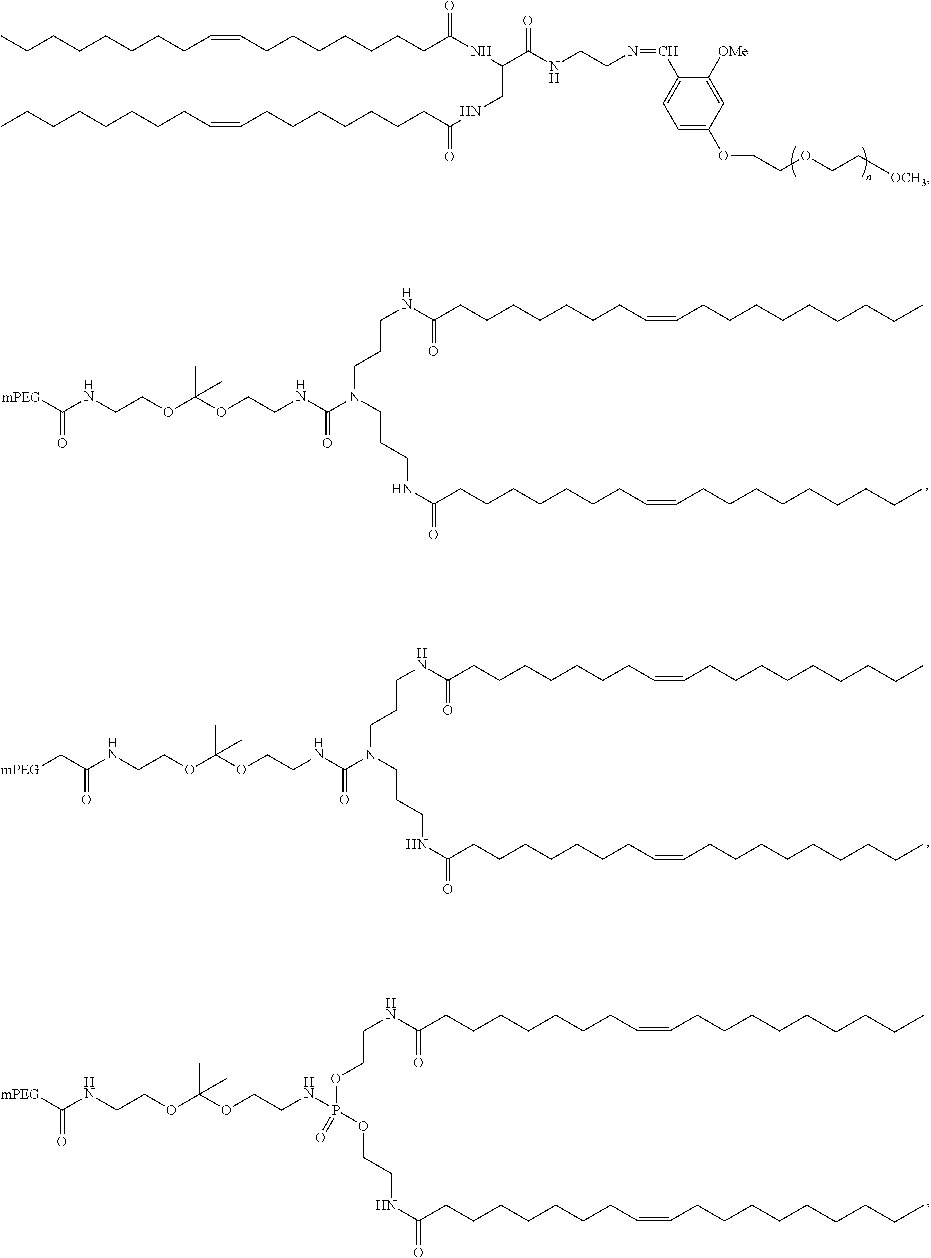 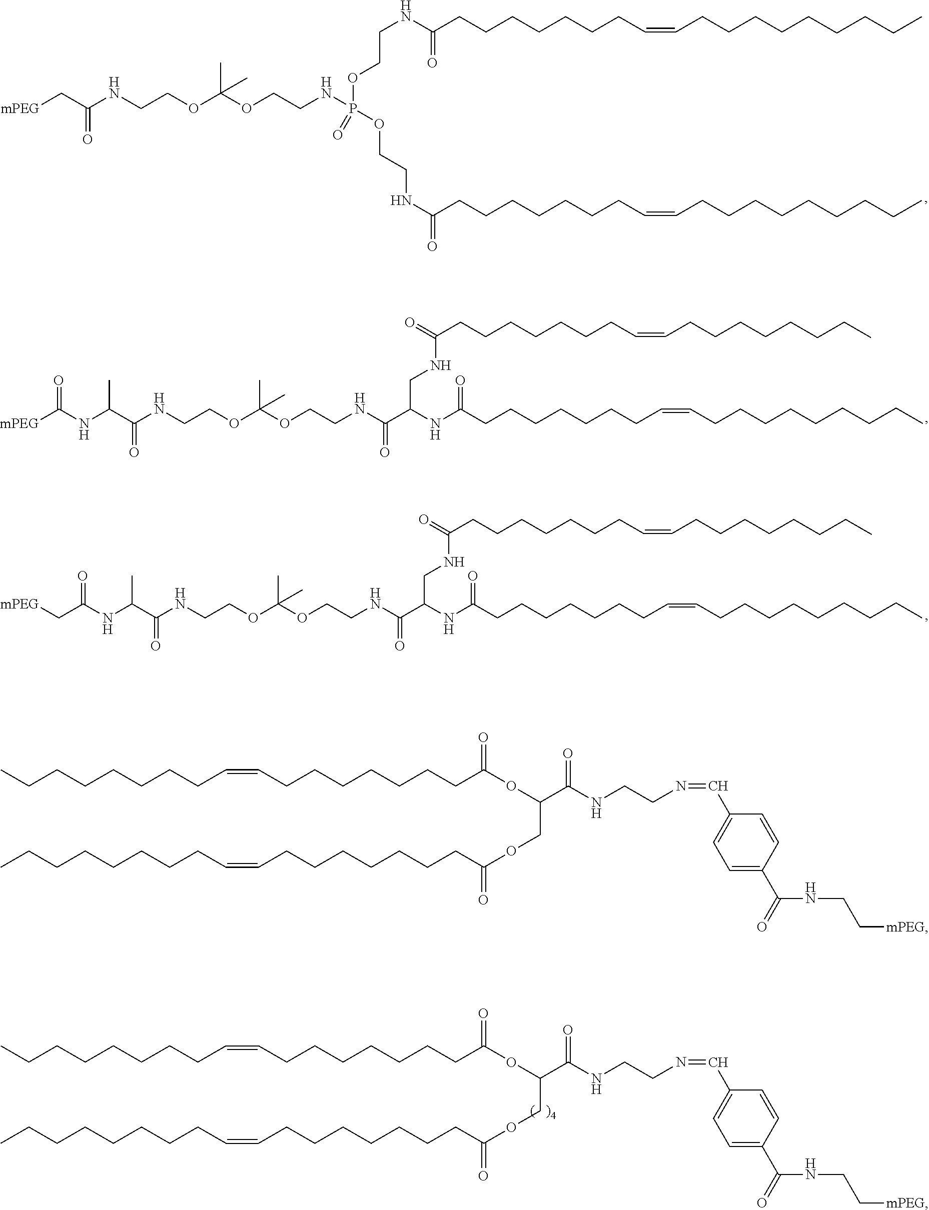 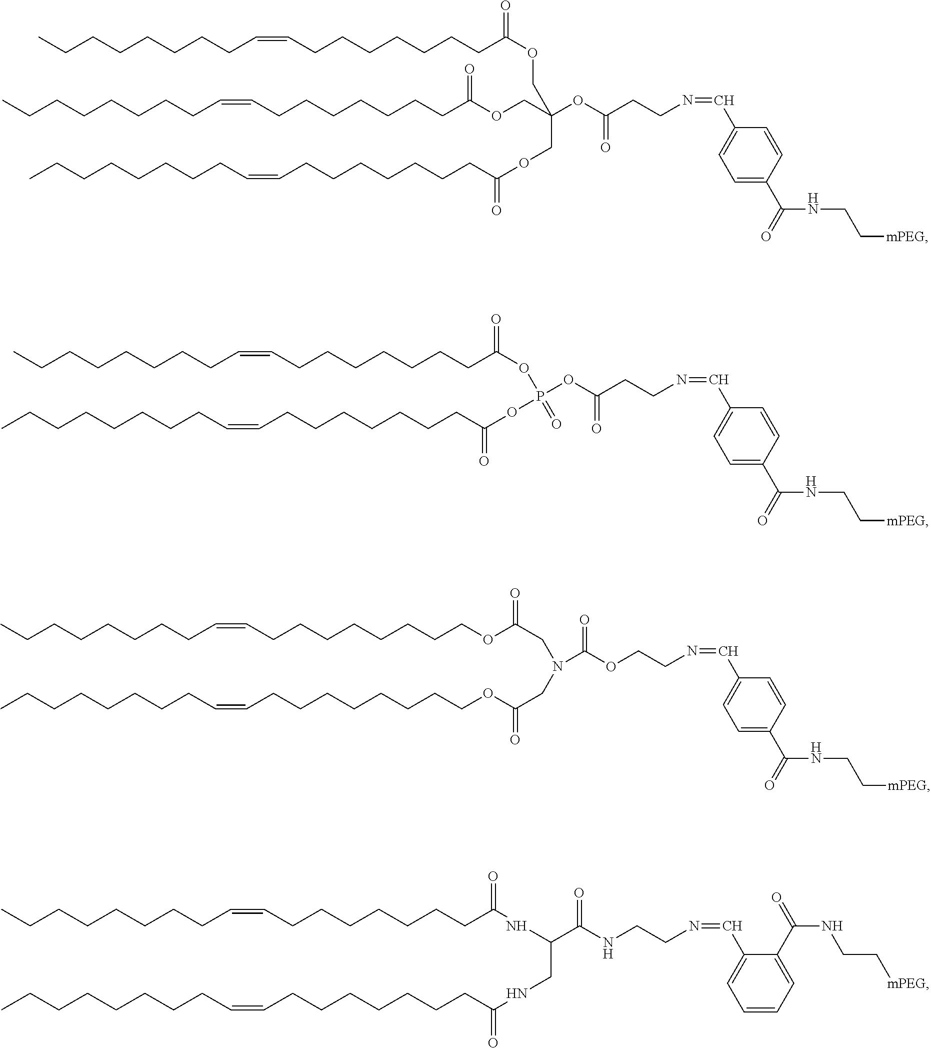 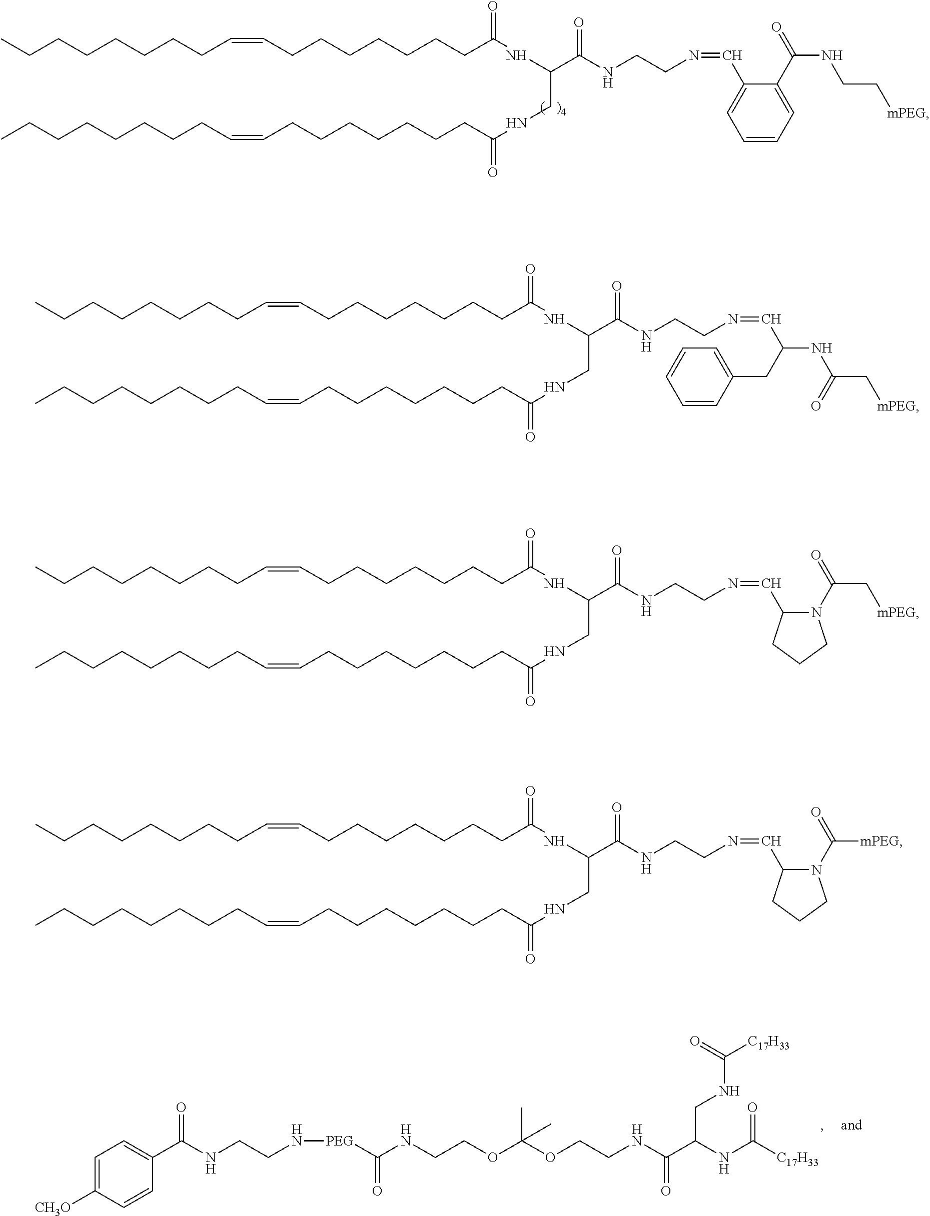 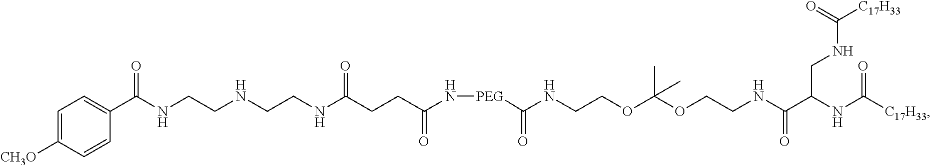
wherein
mPEG is CH₃O(CH₂CH₂O)ₙ—CH₂CH₂O—;
PEG is —(CH₂CH₂O)ₙ—CH₂— or —(CH₂CH₂O)ₙ—CH₂CH₂O—; and
(n) is an integer of from about 10 to about 460.

28. A nanoparticle composition comprising a compound of Formula (I) of claim 1.

29. The nanoparticle composition of claim 28, wherein the compound of Formula (I) is selected from the group consisting of: 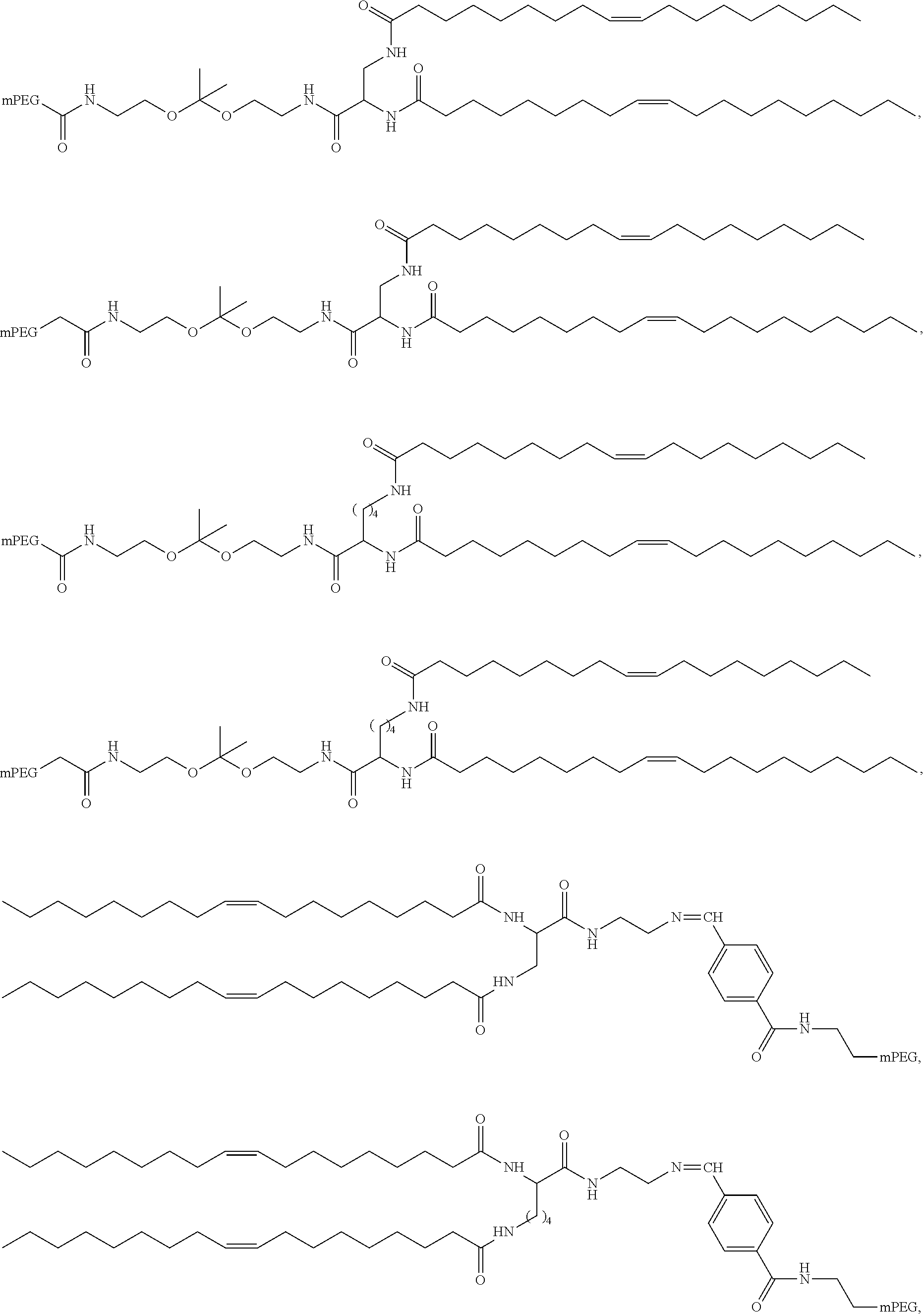 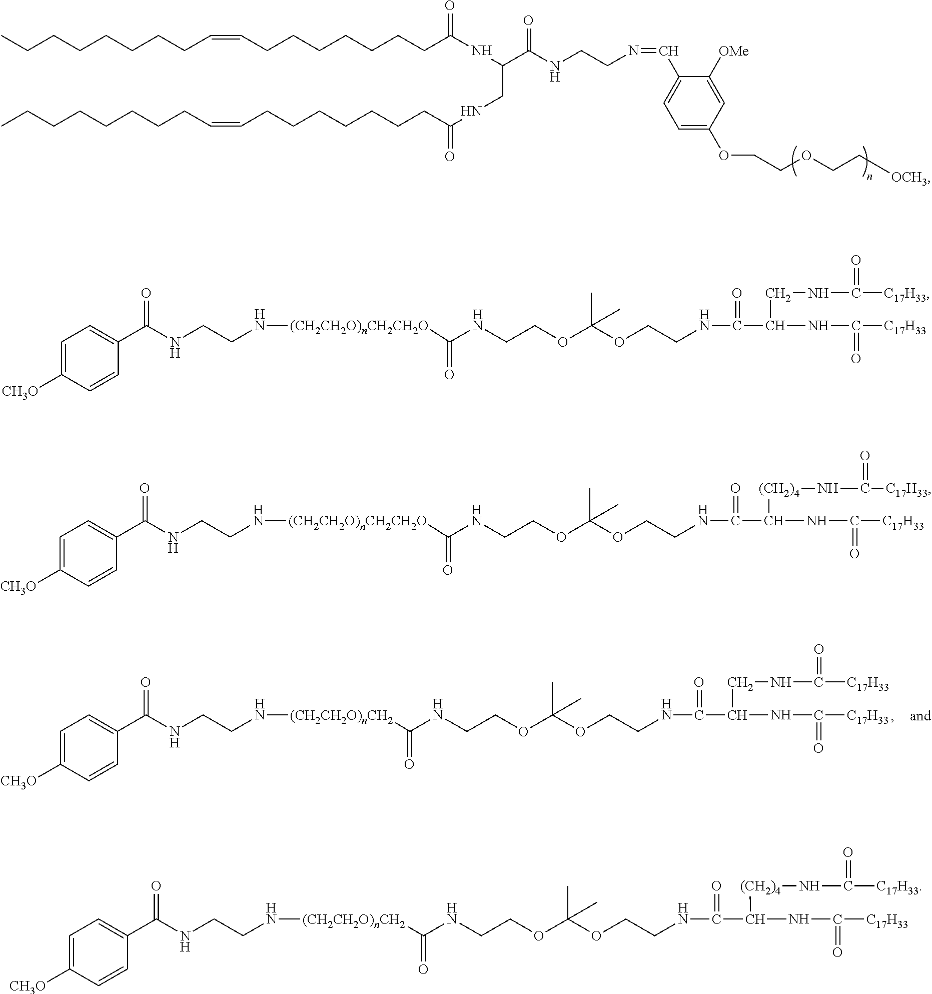
wherein, mPEG is CH₃O(CH₂CH₂O)ₙ—, and (n) is an integer from about 10 to about 460.

30. The nanoparticle composition of claim 28, further comprising a cationic lipid, and fusogenic lipid, wherein the cationic lipid is 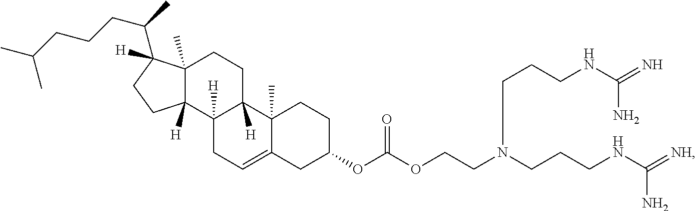
and the fusogenic lipid is selected from the group consisting of DOPE, DOGP, POPC, DSPC, EPC, and combinations thereof.

31.-32. (canceled)

33. The nanoparticle composition of claim 28, further comprising cholesterol.

34. The nanoparticle composition of claim 28, wherein a cationic lipid has a molar ratio ranging from about 10% to about 99.9% of the total lipid present in the nanoparticle composition.

35. (canceled)

36. The nanoparticle composition of claim 33, wherein a molar ratio of a cationic lipid, a non-cholesterol-based fusogenic lipid, a compound of Formula (I), and cholesterol is about 15-25%:20-78%:0-50%:2-10%: of the total lipid present in the nanoparticle composition.

37. The nanoparticle composition of claim 33 selected from the group consisting of
a mixture of a cationic lipid, a diacylphosphatidylethanolamine, a compound of Formula (I), and cholesterol;
a mixture of a cationic lipid, a diacylphosphatidylcholine, a compound of Formula (I), and cholesterol;
a mixture of a cationic lipid, a diacylphosphatidylethanolamine, a diacylphosphatidylcholine, a compound of Formula (I), and cholesterol; and
a mixture of a cationic lipid, a diacylphosphatidylethanolamine, a compound of Formula (I), a PEG conjugated to ceramide (PEG-Cer), and cholesterol.

38. The nanoparticle composition of claim 36, wherein the cationic lipid, DOPE, cholesterol, and a compound of Formula (I) is included in a molar ratio of about 18%:52%: 20%:10% of the total lipid present in the nanoparticle composition, and wherein the cationic lipid is

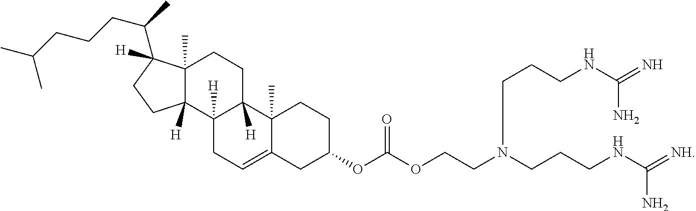

39. The nanoparticle composition of claim 28 comprising nucleic acids encapsulated within the nanoparticle composition.

40. The nanoparticle of claim 39, wherein the nucleic acids is a single stranded or double stranded oligonucleotide.

41. The nanoparticle of claim 39, wherein the nucleic acids is selected from the group consisting of deoxynucleotide, ribonucleotide, locked nucleic acids (LNA), short interfering RNA (siRNA), microRNA (miRNA), aptamers, peptide nucleic acid (PNA), phosphorodiamidate morpholino oligonucleotides (PMO), tricyclo-DNA, double stranded oligonucleotide (decoy ODN), catalytic RNA (RNAi), aptamers, spiegelmers, CpG oligomers and combinations thereof.

42.-45. (canceled)

46. The nanoparticle of claim 40, wherein the oligonucleotide inhibits expression of oncogenes, pro-angiogenesis pathway genes, pro-cell proliferation pathway genes, viral infectious agent genes, and pro-inflammatory pathway genes.

47. The nanoparticle of claim 40, wherein the oligonucleotide is selected from the group consisting of antisense bcl-2 oligonucleotides, antisense HIF-1α oligonucleotides, antisense survivin oligonucleotides, antisense ErbB3 oligonucleotides, antisense PIK3CA oligonucleotides, antisense HSP27 oligonucleotides, antisense androgen receptor oligonucleotides, antisense Gli2 oligonucleotides, and antisense beta-catenin oligonucleotides.

48. The nanoparticle of claim 40, wherein the oligonucleotide comprises eight or more consecutive nucleotides set forth in SEQ ID NO: 1, SEQ ID NOs 2 and 3, SEQ ID NO:3, SEQ ID NO: 4, SEQ ID NO: 5, SEQ ID NO: 6, SEQ ID NO: 7, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17, and each nucleic acid is a naturally occurring or modified nucleic acid.

49. The nanoparticle of claim 40, wherein the charge ratio of the nucleic acids and a cationic lipid ranges from about 1:20 to about 20:1.

50. The nanoparticle of claim 40, wherein the nanoparticle has a size ranging from about 50 nm to about 150 nm.

51. A method of treating disease in a mammal comprising administering a nanoparticle of claim 39 to a mammal in need thereof.

52. (canceled)

53. A method of inhibiting or downregulating a gene expression in human cells or tissues, comprising:
contacting human cells or tissues with a nanoparticle of claim 38.

54. The method of claim 53, wherein the cells or tissues are cancer cells or tissues.

55. (canceled)

56. A method of inhibiting the growth or proliferation of cancer cells comprising:
contacting a cancer cell with a nanoparticle of claim 39.

57. The method of claim 53, further comprising administering an anticancer agent.
